SEQUENCE LISTING (46 sequences)

SEQ ID NO: 1 - DNA, 891 bp, Artificial Sequence
  NFAT/GFP
ggaggaaaaactgtttcatacagaaggcgtggaggaaaaactgtttcatacagaaggcgtggaggaaaaactgtttcatacagaaggcgtgggcccaggtaggcgtgtacggtgggaggtctatataagcagagctggtttagtgaaccgtcagatcgcggccgcgaattcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 2 - DNA, 6867 bp, Artificial Sequence
  NGFR/NFAT/GFP vector
taaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggcgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggcgattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattaagatcttggccacaaccaaccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggccttgtggcctacatagccttcaagaggtggaacagtcatcgctatcctcgaggtcaccgcggtctagagtccacctgcagccaagcttatcgataaaccggtgggaggaaaaactgtttcatacagaaggcgtggaggaaaaactgtttcatacagaaggcgtggaggaaaaactgtttcatacagaaggcgtgggcccaggtaggcgtgtacggtgggaggtctatataagcagagctggtttagtgaaccgtcagatcgcggccgcgaattcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagtcgacctgcagccaagcttatcgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcatgggtaacagtttcttgaagttggagaacaacattctgagggtaggagtcgaatattaagtaatcctgactcaattagccactgttttgaatccacatactccaatactcctgaaatagttcattatggacagcgcagaaagagctggggagaattgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggacta

SEQ ID NO: 3 - DNA, 2876 bp, Artificial Sequence
  pAAV
tttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatt

SEQ ID NO: 4 - DNA, 20 bp, Artificial Sequence
  Murine PD-1 sgRNA
gccaggggcucugggcaugu

SEQ ID NO: 5 - DNA, 20 bp, Artificial Sequence
  Murine NR4A2 sgRNA
cugcgccugaacacaaggca

SEQ ID NO: 6 - DNA, 20 bp, Artificial Sequence
  Human PD-1 sgRNA
uccaggcaugcagaucccac

SEQ ID NO: 7 - DNA, 20 bp, Artificial Sequence
  Human NR4A2 sgRNA
gccugaacacaaggcauggc

SEQ ID NO: 8 - DNA, 1702 bp, Artificial Sequence
  Murine PD-1/GFP HDR template
gcggccgcgaggggctagaaatggagaggaccccatagcaggacaagaggcaaggacagctagtcagagagaaccccccctctctgctccccaatctctcactagtcccttacctgctcctcccaggcatcgttccctcccactcccctcccccttccatgcccctcccccacctctagttgcctgttctcccacccttgtggaggtggaggaagagggggcgggagccaagaacaggtctcctccctccaacatgacctgggacagtttcctttccgctacagacaactctgcctgagcagcggggaggaggaagaggagactgctactgaaggcgacactgccaggggctctgggcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatctagagggccctattgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtgggtccggcaggtaccctggtcattcacttgggctgtgctgcagttgagctggcaatcagggtggcttctaggtatgtggctcctagggatgtgggatcaattggggttggtggctggggcagggcttgaagaaacgttcaccctcggttcctattactggaggggaggaacacctgtgtccctggttacacggattggctcctggttacacagattggctttaggtgggttttacaggtgaaatgccccctggggctcaaaatccaccccctccttgcttttagacagacagaacatttatctaacgtcagttgatcttctgtgtccctctctgtgaagcgatggtaggcggccgc

SEQ ID NO: 9 - DNA, 1701 bp, Artificial Sequence
  Murine NR4A2/GFP HDR template
gcggccgcgcttctagaaatcctgtcgaccctgagcttcaaagaagaagctcatcagagtgggactgtctgcgggaagggggtggagcgcggggggggggcgtttggaaatgagtgtagaccctcaacagctttccagctctgggtcgtcccgggatcagccctttcctttcttcatcgctgttccacctcttttgcccttccccctgcatccctaaacccccatcctctccccgcctccctccacccccaaccccgacgccgcgggctgccggtgtagccccgggtgtagaccgagcccggaagaaagtgttcagttgaccaggctgagtgtatatcaccctgtttcgtttccagccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatctagagggccctattgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgaccttgtgttcaggcgcagtatgggtcctcgcctcaaggagccagccccgcttctcagagctacagttaccactcttcgggagaatacagctccgatttcttaactccagagtttgtcaagtttagcatggacctcaccaacactgaaattactgccaccacttctctccccagcttcagtacctttatggacaactacagcacaggctacgacgtcaagccaccttgcttgtaccaaatgcccctgtccggacagcagtcctccattaaggtagaagacattcagatgcacaactaccagcaacacagccacctgccccctcagtccgaggagatgatgccacacagcggcggccgc

SEQ ID NO: 10 - DNA, 1363 bp, Artificial Sequence
  Murine PD-1/CXCL9 HDR template
gcggccgcgaggggctagaaatggagaggaccccatagcaggacaagaggcaaggacagctagtcagagagaaccccccctctctgctccccaatctctcactagtcccttacctgctcctcccaggcatcgttccctcccactcccctcccccttccatgcccctcccccacctctagttgcctgttctcccacccttgtggaggtggaggaagagggggcgggagccaagaacaggtctcctccctccaacatgacctgggacagtttcctttccgctacagacaactctgcctgagcagcggggaggaggaagaggagactgctactgaaggcgacactgccaggggctctgggcatgaagtccgctgttcttttcctcttgggcatcatcttcctggagcagtgtggagttcgaggaaccctagtgataaggaatgcacgatgctcctgcatcagcaccagccgaggcacgatccactacaaatccctcaaagacctcaaacagtttgccccaagccccaattgcaacaaaactgaaatcattgctacactgaagaacggagatcaaacctgcctagatccggactcggcaaatgtgaagaagctgatgaaagaatgggaaaagaagatcagccaaaagaaaaagcaaaagagggggaaaaaacatcaaaagaacatgaaaaacagaaaacccaaaacaccccaaagtcgtcgtcgttcaaggaagactacataatctagagggccctattgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtgggtccggcaggtaccctggtcattcacttgggctgtgctgcagttgagctggcaatcagggtggcttctaggtatgtggctcctagggatgtgggatcaattggggttggtggctggggcagggcttgaagaaacgttcaccctcggttcctattactggaggggaggaacacctgtgtccctggttacacggattggctcctggttacacagattggctttaggtgggttttacaggtgaaatgccccctggggctcaaaatccaccccctccttgcttttagacagacagaacatttatctaacgtcagttgatcttctgtgtccctctctgtgaagcgatggtaggcggccgc

SEQ ID NO: 11 - DNA, 1362 bp, Artificial Sequence
  Murine NR4A2/CXCL9 HDR template
gcggccgcgcttctagaaatcctgtcgaccctgagcttcaaagaagaagctcatcagagtgggactgtctgcgggaagggggtggagcgcggggggggggcgtttggaaatgagtgtagaccctcaacagctttccagctctgggtcgtcccgggatcagccctttcctttcttcatcgctgttccacctcttttgcccttccccctgcatccctaaacccccatcctctccccgcctccctccacccccaaccccgacgccgcgggctgccggtgtagccccgggtgtagaccgagcccggaagaaagtgttcagttgaccaggctgagtgtatatcaccctgtttcgtttccagccatgaagtccgctgttcttttcctcttgggcatcatcttcctggagcagtgtggagttcgaggaaccctagtgataaggaatgcacgatgctcctgcatcagcaccagccgaggcacgatccactacaaatccctcaaagacctcaaacagtttgccccaagccccaattgcaacaaaactgaaatcattgctacactgaagaacggagatcaaacctgcctagatccggactcggcaaatgtgaagaagctgatgaaagaatgggaaaagaagatcagccaaaagaaaaagcaaaagagggggaaaaaacatcaaaagaacatgaaaaacagaaaacccaaaacaccccaaagtcgtcgtcgttcaaggaagactacataatctagagggccctattgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgaccttgtgttcaggcgcagtatgggtcctcgcctcaaggagccagccccgcttctcagagctacagttaccactcttcgggagaatacagctccgatttcttaactccagagtttgtcaagtttagcatggacctcaccaacactgaaattactgccaccacttctctccccagcttcagtacctttatggacaactacagcacaggctacgacgtcaagccaccttgcttgtaccaaatgcccctgtccggacagcagtcctccattaaggtagaagacattcagatgcacaactaccagcaacacagccacctgccccctcagtccgaggagatgatgccacacagcggcggccgc

SEQ ID NO: 12 - DNA, 1279 bp, Artificial Sequence
  Murine PD-1/CXCL10 HDR template
gcggccgcgaggggctagaaatggagaggaccccatagcaggacaagaggcaaggacagctagtcagagagaaccccccctctctgctccccaatctctcactagtcccttacctgctcctcccaggcatcgttccctcccactcccctcccccttccatgcccctcccccacctctagttgcctgttctcccacccttgtggaggtggaggaagagggggcgggagccaagaacaggtctcctccctccaacatgacctgggacagtttcctttccgctacagacaactctgcctgagcagcggggaggaggaagaggagactgctactgaaggcgacactgccaggggctctgggcatgaacccaagtgctgccgtcattttctgcctcatcctgctgggtctgagtgggactcaagggatccctctcgcaaggacggtccgctgcaactgcatccatatcgatgacgggccagtgagaatgagggccatagggaagcttgaaatcatccctgcgagcctatcctgcccacgtgttgagatcattgccacgatgaaaaagaatgatgagcagagatgtctgaatccggaatctaagaccatcaagaatttaatgaaagcgtttagccaaaaaaggtctaaaagggctccttaatctagagggccctattgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtgggtccggcaggtaccctggtcattcacttgggctgtgctgcagttgagctggcaatcagggtggcttctaggtatgtggctcctagggatgtgggatcaattggggttggtggctggggcagggcttgaagaaacgttcaccctcggttcctattactggaggggaggaacacctgtgtccctggttacacggattggctcctggttacacagattggctttaggtgggttttacaggtgaaatgccccctggggctcaaaatccaccccctccttgcttttagacagacagaacatttatctaacgtcagttgatcttctgtgtccctctctgtgaagcgatggtaggcggccgc

SEQ ID NO: 13 - DNA, 1278 bp, Artificial Sequence
  Murine NR4A2/CXCL10 HDR template
gcggccgcgcttctagaaatcctgtcgaccctgagcttcaaagaagaagctcatcagagtgggactgtctgcgggaagggggtggagcgcggggggggggcgtttggaaatgagtgtagaccctcaacagctttccagctctgggtcgtcccgggatcagccctttcctttcttcatcgctgttccacctcttttgcccttccccctgcatccctaaacccccatcctctccccgcctccctccacccccaaccccgacgccgcgggctgccggtgtagccccgggtgtagaccgagcccggaagaaagtgttcagttgaccaggctgagtgtatatcaccctgtttcgtttccagccatgaacccaagtgctgccgtcattttctgcctcatcctgctgggtctgagtgggactcaagggatccctctcgcaaggacggtccgctgcaactgcatccatatcgatgacgggccagtgagaatgagggccatagggaagcttgaaatcatccctgcgagcctatcctgcccacgtgttgagatcattgccacgatgaaaaagaatgatgagcagagatgtctgaatccggaatctaagaccatcaagaatttaatgaaagcgtttagccaaaaaaggtctaaaagggctccttaatctagagggccctattgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgaccttgtgttcaggcgcagtatgggtcctcgcctcaaggagccagccccgcttctcagagctacagttaccactcttcgggagaatacagctccgatttcttaactccagagtttgtcaagtttagcatggacctcaccaacactgaaattactgccaccacttctctccccagcttcagtacctttatggacaactacagcacaggctacgacgtcaagccaccttgcttgtaccaaatgcccctgtccggacagcagtcctccattaaggtagaagacattcagatgcacaactaccagcaacacagccacctgccccctcagtccgaggagatgatgccacacagcggcggccgc

SEQ ID NO: 14 - DNA, 1723 bp, Artificial Sequence
  Murine PD-1/ CXCL9-P2A-CXCL10 HDR template
gcggccgcgaggggctagaaatggagaggaccccatagcaggacaagaggcaaggacagctagtcagagagaaccccccctctctgctccccaatctctcactagtcccttacctgctcctcccaggcatcgttccctcccactcccctcccccttccatgcccctcccccacctctagttgcctgttctcccacccttgtggaggtggaggaagagggggcgggagccaagaacaggtctcctccctccaacatgacctgggacagtttcctttccgctacagacaactctgcctgagcagcggggaggaggaagaggagactgctactgaaggcgacactgccaggggctctgggcatgaagtccgctgttcttttcctcttgggcatcatcttcctggagcagtgtggagttcgaggaaccctagtgataaggaatgcacgatgctcctgcatcagcaccagccgaggcacgatccactacaaatccctcaaagacctcaaacagtttgccccaagccccaattgcaacaaaactgaaatcattgctacactgaagaacggagatcaaacctgcctagatccggactcggcaaatgtgaagaagctgatgaaagaatgggaaaagaagatcagccaaaagaaaaagcaaaagagggggaaaaaacatcaaaagaacatgaaaaacagaaaacccaaaacaccccaaagtcgtcgtcgttcaaggaagactacaggatccggagctactaacttcagcctgctgaagcaggctggcgacgtggaggagaaccctggacctatgaacccaagtgctgccgtcattttctgcctcatcctgctgggtctgagtgggactcaagggatccctctcgcaaggacggtccgctgcaactgcatccatatcgatgacgggccagtgagaatgagggccatagggaagcttgaaatcatccctgcgagcctatcctgcccacgtgttgagatcattgccacgatgaaaaagaatgatgagcagagatgtctgaatccggaatctaagaccatcaagaatttaatgaaagcgtttagccaaaaaaggtctaaaagggctccttaatctagagggccctattgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtgggtccggcaggtaccctggtcattcacttgggctgtgctgcagttgagctggcaatcagggtggcttctaggtatgtggctcctagggatgtgggatcaattggggttggtggctggggcagggcttgaagaaacgttcaccctcggttcctattactggaggggaggaacacctgtgtccctggttacacggattggctcctggttacacagattggctttaggtgggttttacaggtgaaatgccccctggggctcaaaatccaccccctccttgcttttagacagacagaacatttatctaacgtcagttgatcttctgtgtccctctctgtgaagcgatggtaggcggccgc

SEQ ID NO: 15 - DNA, 1722 bp, Artificial Sequence
  Murine NR4A2/CXCL9-P2A-CXCL10 HDR template
gcggccgcgcttctagaaatcctgtcgaccctgagcttcaaagaagaagctcatcagagtgggactgtctgcgggaagggggtggagcgcggggggggggcgtttggaaatgagtgtagaccctcaacagctttccagctctgggtcgtcccgggatcagccctttcctttcttcatcgctgttccacctcttttgcccttccccctgcatccctaaacccccatcctctccccgcctccctccacccccaaccccgacgccgcgggctgccggtgtagccccgggtgtagaccgagcccggaagaaagtgttcagttgaccaggctgagtgtatatcaccctgtttcgtttccagccatgaagtccgctgttcttttcctcttgggcatcatcttcctggagcagtgtggagttcgaggaaccctagtgataaggaatgcacgatgctcctgcatcagcaccagccgaggcacgatccactacaaatccctcaaagacctcaaacagtttgccccaagccccaattgcaacaaaactgaaatcattgctacactgaagaacggagatcaaacctgcctagatccggactcggcaaatgtgaagaagctgatgaaagaatgggaaaagaagatcagccaaaagaaaaagcaaaagagggggaaaaaacatcaaaagaacatgaaaaacagaaaacccaaaacaccccaaagtcgtcgtcgttcaaggaagactacaggatccggagctactaacttcagcctgctgaagcaggctggcgacgtggaggagaaccctggacctatgaacccaagtgctgccgtcattttctgcctcatcctgctgggtctgagtgggactcaagggatccctctcgcaaggacggtccgctgcaactgcatccatatcgatgacgggccagtgagaatgagggccatagggaagcttgaaatcatccctgcgagcctatcctgcccacgtgttgagatcattgccacgatgaaaaagaatgatgagcagagatgtctgaatccggaatctaagaccatcaagaatttaatgaaagcgtttagccaaaaaaggtctaaaagggctccttaatctagagggccctattgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgaccttgtgttcaggcgcagtatgggtcctcgcctcaaggagccagccccgcttctcagagctacagttaccactcttcgggagaatacagctccgatttcttaactccagagtttgtcaagtttagcatggacctcaccaacactgaaattactgccaccacttctctccccagcttcagtacctttatggacaactacagcacaggctacgacgtcaagccaccttgcttgtaccaaatgcccctgtccggacagcagtcctccattaaggtagaagacattcagatgcacaactaccagcaacacagccacctgccccctcagtccgaggagatgatgccacacagcggcggccgc

SEQ ID NO: 16 - DNA, 1690 bp, Artificial Sequence
  Murine PD-1/TNF HDR template
gcggccgcgaggggctagaaatggagaggaccccatagcaggacaagaggcaaggacagctagtcagagagaaccccccctctctgctccccaatctctcactagtcccttacctgctcctcccaggcatcgttccctcccactcccctcccccttccatgcccctcccccacctctagttgcctgttctcccacccttgtggaggtggaggaagagggggcgggagccaagaacaggtctcctccctccaacatgacctgggacagtttcctttccgctacagacaactctgcctgagcagcggggaggaggaagaggagactgctactgaaggcgacactgccaggggctctgggcatgagcacagaaagcatgatccgcgacgtggaactggcagaagaggcactcccccaaaagatggggggcttccagaactccaggcggtgcctatgtctcagcctcttctcattcctgcttgtggcaggggccaccacgctcttctgtctactgaacttcggggtgatcggtccccaaagggatgagaagttcccaaatggcctccctctcatcagttctatggcccagaccctcacactcagatcatcttctcaaaattcgagtgacaagcctgtagcccacgtcgtagcaaaccaccaagtggaggagcagctggagtggctgagccagcgcgccaacgccctcctggccaacggcatggatctcaaagacaaccaactagtggtgccagccgatgggttgtaccttgtctactcccaggttctcttcaagggacaaggctgccccgactacgtgctcctcacccacaccgtcagccgatttgctatctcataccaggagaaagtcaacctcctctctgccgtcaagagcccctgccccaaggacacccctgagggggctgagctcaaaccctggtatgagcccatatacctgggaggagtcttccagctggagaagggggaccaactcagcgctgaggtcaatctgcccaagtacttagactttgcggagtccgggcaggtctactttggagtcattgctctgtgatctagagggccctattgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtgggtccggcaggtaccctggtcattcacttgggctgtgctgcagttgagctggcaatcagggtggcttctaggtatgtggctcctagggatgtgggatcaattggggttggtggctggggcagggcttgaagaaacgttcaccctcggttcctattactggaggggaggaacacctgtgtccctggttacacggattggctcctggttacacagattggctttaggtgggttttacaggtgaaatgccccctggggctcaaaatccaccccctccttgcttttagacagacagaacatttatctaacgtcagttgatcttctgtgtccctctctgtgaagcgatggtaggcggccgc

SEQ ID NO: 17 - DNA, 1689 bp, Artificial Sequence
  Murine NR4A2/TNF HDR template
gcggccgcgcttctagaaatcctgtcgaccctgagcttcaaagaagaagctcatcagagtgggactgtctgcgggaagggggtggagcgcggggggggggcgtttggaaatgagtgtagaccctcaacagctttccagctctgggtcgtcccgggatcagccctttcctttcttcatcgctgttccacctcttttgcccttccccctgcatccctaaacccccatcctctccccgcctccctccacccccaaccccgacgccgcgggctgccggtgtagccccgggtgtagaccgagcccggaagaaagtgttcagttgaccaggctgagtgtatatcaccctgtttcgtttccagccatgagcacagaaagcatgatccgcgacgtggaactggcagaagaggcactcccccaaaagatggggggcttccagaactccaggcggtgcctatgtctcagcctcttctcattcctgcttgtggcaggggccaccacgctcttctgtctactgaacttcggggtgatcggtccccaaagggatgagaagttcccaaatggcctccctctcatcagttctatggcccagaccctcacactcagatcatcttctcaaaattcgagtgacaagcctgtagcccacgtcgtagcaaaccaccaagtggaggagcagctggagtggctgagccagcgcgccaacgccctcctggccaacggcatggatctcaaagacaaccaactagtggtgccagccgatgggttgtaccttgtctactcccaggttctcttcaagggacaaggctgccccgactacgtgctcctcacccacaccgtcagccgatttgctatctcataccaggagaaagtcaacctcctctctgccgtcaagagcccctgccccaaggacacccctgagggggctgagctcaaaccctggtatgagcccatatacctgggaggagtcttccagctggagaagggggaccaactcagcgctgaggtcaatctgcccaagtacttagactttgcggagtccgggcaggtctactttggagtcattgctctgtgatctagagggccctattgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgaccttgtgttcaggcgcagtatgggtcctcgcctcaaggagccagccccgcttctcagagctacagttaccactcttcgggagaatacagctccgatttcttaactccagagtttgtcaagtttagcatggacctcaccaacactgaaattactgccaccacttctctccccagcttcagtacctttatggacaactacagcacaggctacgacgtcaagccaccttgcttgtaccaaatgcccctgtccggacagcagtcctccattaaggtagaagacattcagatgcacaactaccagcaacacagccacctgccccctcagtccgaggagatgatgccacacagcggcggccgc

SEQ ID NO: 18 - DNA, 1450 bp, Artificial Sequence
  Murine PD-1/IFNg HDR template
gcggccgcgaggggctagaaatggagaggaccccatagcaggacaagaggcaaggacagctagtcagagagaaccccccctctctgctccccaatctctcactagtcccttacctgctcctcccaggcatcgttccctcccactcccctcccccttccatgcccctcccccacctctagttgcctgttctcccacccttgtggaggtggaggaagagggggcgggagccaagaacaggtctcctccctccaacatgacctgggacagtttcctttccgctacagacaactctgcctgagcagcggggaggaggaagaggagactgctactgaaggcgacactgccaggggctctgggcatgaacgctacacactgcatcttggctttgcagctcttcctcatggctgtttctggctgttactgccacggcacagtcattgaaagcctagaaagtctgaataactattttaactcaagtggcatagatgtggaagaaaagagtctcttcttggatatctggaggaactggcaaaaggatggtgacatgaaaatcctgcagagccagattatctctttctacctcagactctttgaagtcttgaaagacaatcaggccatcagcaacaacataagcgtcattgaatcacacctgattactaccttcttcagcaacagcaaggcgaaaaaggatgcattcatgagtattgccaagtttgaggtcaacaacccacaggtccagcgccaagcattcaatgagctcatccgagtggtccaccagctgttgccggaatccagcctcaggaagcggaaaaggagtcgctgctgatctagagggccctattgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtgggtccggcaggtaccctggtcattcacttgggctgtgctgcagttgagctggcaatcagggtggcttctaggtatgtggctcctagggatgtgggatcaattggggttggtggctggggcagggcttgaagaaacgttcaccctcggttcctattactggaggggaggaacacctgtgtccctggttacacggattggctcctggttacacagattggctttaggtgggttttacaggtgaaatgccccctggggctcaaaatccaccccctccttgcttttagacagacagaacatttatctaacgtcagttgatcttctgtgtccctctctgtgaagcgatggtaggcggccgc

SEQ ID NO: 19 - DNA, 1449 bp, Artificial Sequence
  Murine NR4A2/IFNg HDR template
gcggccgcgcttctagaaatcctgtcgaccctgagcttcaaagaagaagctcatcagagtgggactgtctgcgggaagggggtggagcgcggggggggggcgtttggaaatgagtgtagaccctcaacagctttccagctctgggtcgtcccgggatcagccctttcctttcttcatcgctgttccacctcttttgcccttccccctgcatccctaaacccccatcctctccccgcctccctccacccccaaccccgacgccgcgggctgccggtgtagccccgggtgtagaccgagcccggaagaaagtgttcagttgaccaggctgagtgtatatcaccctgtttcgtttccagccatgaacgctacacactgcatcttggctttgcagctcttcctcatggctgtttctggctgttactgccacggcacagtcattgaaagcctagaaagtctgaataactattttaactcaagtggcatagatgtggaagaaaagagtctcttcttggatatctggaggaactggcaaaaggatggtgacatgaaaatcctgcagagccagattatctctttctacctcagactctttgaagtcttgaaagacaatcaggccatcagcaacaacataagcgtcattgaatcacacctgattactaccttcttcagcaacagcaaggcgaaaaaggatgcattcatgagtattgccaagtttgaggtcaacaacccacaggtccagcgccaagcattcaatgagctcatccgagtggtccaccagctgttgccggaatccagcctcaggaagcggaaaaggagtcgctgctgatctagagggccctattgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgaccttgtgttcaggcgcagtatgggtcctcgcctcaaggagccagccccgcttctcagagctacagttaccactcttcgggagaatacagctccgatttcttaactccagagtttgtcaagtttagcatggacctcaccaacactgaaattactgccaccacttctctccccagcttcagtacctttatggacaactacagcacaggctacgacgtcaagccaccttgcttgtaccaaatgcccctgtccggacagcagtcctccattaaggtagaagacattcagatgcacaactaccagcaacacagccacctgccccctcagtccgaggagatgatgccacacagcggcggccgc

SEQ ID NO: 20 - DNA, 2221 bp, Artificial Sequence
  Murine PD-1/TNF-P2A-INFg HDR template
gcggccgcgaggggctagaaatggagaggaccccatagcaggacaagaggcaaggacagctagtcagagagaaccccccctctctgctccccaatctctcactagtcccttacctgctcctcccaggcatcgttccctcccactcccctcccccttccatgcccctcccccacctctagttgcctgttctcccacccttgtggaggtggaggaagagggggcgggagccaagaacaggtctcctccctccaacatgacctgggacagtttcctttccgctacagacaactctgcctgagcagcggggaggaggaagaggagactgctactgaaggcgacactgccaggggctctgggcatgagcacagaaagcatgatccgcgacgtggaactggcagaagaggcactcccccaaaagatggggggcttccagaactccaggcggtgcctatgtctcagcctcttctcattcctgcttgtggcaggggccaccacgctcttctgtctactgaacttcggggtgatcggtccccaaagggatgagaagttcccaaatggcctccctctcatcagttctatggcccagaccctcacactcagatcatcttctcaaaattcgagtgacaagcctgtagcccacgtcgtagcaaaccaccaagtggaggagcagctggagtggctgagccagcgcgccaacgccctcctggccaacggcatggatctcaaagacaaccaactagtggtgccagccgatgggttgtaccttgtctactcccaggttctcttcaagggacaaggctgccccgactacgtgctcctcacccacaccgtcagccgatttgctatctcataccaggagaaagtcaacctcctctctgccgtcaagagcccctgccccaaggacacccctgagggggctgagctcaaaccctggtatgagcccatatacctgggaggagtcttccagctggagaagggggaccaactcagcgctgaggtcaatctgcccaagtacttagactttgcggagtccgggcaggtctactttggagtcattgctctgggatccggagctactaacttcagcctgctgaagcaggctggcgacgtggaggagaaccctggacctatgaacgctacacactgcatcttggctttgcagctcttcctcatggctgtttctggctgttactgccacggcacagtcattgaaagcctagaaagtctgaataactattttaactcaagtggcatagatgtggaagaaaagagtctcttcttggatatctggaggaactggcaaaaggatggtgacatgaaaatcctgcagagccagattatctctttctacctcagactctttgaagtcttgaaagacaatcaggccatcagcaacaacataagcgtcattgaatcacacctgattactaccttcttcagcaacagcaaggcgaaaaaggatgcattcatgagtattgccaagtttgaggtcaacaacccacaggtccagcgccaagcattcaatgagctcatccgagtggtccaccagctgttgccggaatccagcctcaggaagcggaaaaggagtcgctgctgatctagagggccctattgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtgggtccggcaggtaccctggtcattcacttgggctgtgctgcagttgagctggcaatcagggtggcttctaggtatgtggctcctagggatgtgggatcaattggggttggtggctggggcagggcttgaagaaacgttcaccctcggttcctattactggaggggaggaacacctgtgtccctggttacacggattggctcctggttacacagattggctttaggtgggttttacaggtgaaatgccccctggggctcaaaatccaccccctccttgcttttagacagacagaacatttatctaacgtcagttgatcttctgtgtccctctctgtgaagcgatggtaggcggccgc

SEQ ID NO: 21 - DNA, 2220 bp, Artificial Sequence
  Murine NR4A2/TNF-P2A-INFg HDR template
gcggccgcgcttctagaaatcctgtcgaccctgagcttcaaagaagaagctcatcagagtgggactgtctgcgggaagggggtggagcgcggggggggggcgtttggaaatgagtgtagaccctcaacagctttccagctctgggtcgtcccgggatcagccctttcctttcttcatcgctgttccacctcttttgcccttccccctgcatccctaaacccccatcctctccccgcctccctccacccccaaccccgacgccgcgggctgccggtgtagccccgggtgtagaccgagcccggaagaaagtgttcagttgaccaggctgagtgtatatcaccctgtttcgtttccagccatgagcacagaaagcatgatccgcgacgtggaactggcagaagaggcactcccccaaaagatggggggcttccagaactccaggcggtgcctatgtctcagcctcttctcattcctgcttgtggcaggggccaccacgctcttctgtctactgaacttcggggtgatcggtccccaaagggatgagaagttcccaaatggcctccctctcatcagttctatggcccagaccctcacactcagatcatcttctcaaaattcgagtgacaagcctgtagcccacgtcgtagcaaaccaccaagtggaggagcagctggagtggctgagccagcgcgccaacgccctcctggccaacggcatggatctcaaagacaaccaactagtggtgccagccgatgggttgtaccttgtctactcccaggttctcttcaagggacaaggctgccccgactacgtgctcctcacccacaccgtcagccgatttgctatctcataccaggagaaagtcaacctcctctctgccgtcaagagcccctgccccaaggacacccctgagggggctgagctcaaaccctggtatgagcccatatacctgggaggagtcttccagctggagaagggggaccaactcagcgctgaggtcaatctgcccaagtacttagactttgcggagtccgggcaggtctactttggagtcattgctctgggatccggagctactaacttcagcctgctgaagcaggctggcgacgtggaggagaaccctggacctatgaacgctacacactgcatcttggctttgcagctcttcctcatggctgtttctggctgttactgccacggcacagtcattgaaagcctagaaagtctgaataactattttaactcaagtggcatagatgtggaagaaaagagtctcttcttggatatctggaggaactggcaaaaggatggtgacatgaaaatcctgcagagccagattatctctttctacctcagactctttgaagtcttgaaagacaatcaggccatcagcaacaacataagcgtcattgaatcacacctgattactaccttcttcagcaacagcaaggcgaaaaaggatgcattcatgagtattgccaagtttgaggtcaacaacccacaggtccagcgccaagcattcaatgagctcatccgagtggtccaccagctgttgccggaatccagcctcaggaagcggaaaaggagtcgctgctgatctagagggccctattgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgaccttgtgttcaggcgcagtatgggtcctcgcctcaaggagccagccccgcttctcagagctacagttaccactcttcgggagaatacagctccgatttcttaactccagagtttgtcaagtttagcatggacctcaccaacactgaaattactgccaccacttctctccccagcttcagtacctttatggacaactacagcacaggctacgacgtcaagccaccttgcttgtaccaaatgcccctgtccggacagcagtcctccattaaggtagaagacattcagatgcacaactaccagcaacacagccacctgccccctcagtccgaggagatgatgccacacagcggcggccgc

SEQ ID NO: 22 - DNA, 2167 bp, Artificial Sequence
  Human PD-1/NGFR HDR template
gcggccgctcactgcagcccaggaacctgagcccagagggggccacccaccttccccaggcagggaggcccggcccccagggagatgggggggatgggggaggagaagggcctgcccccacccggcagcctcaggaggggcagctcgggcgggatatggaaagaggccacagcagtgagcagagacacagaggaggaaggggccctgagctggggagacccccacggggtagggcgtgggggccacgggcccacctcctccccatctcctctgtctccctgtctctgtctctctctccctcccccaccctctccccagtcctaccccctcctcacccctcctcccccagcactgcctctgtcactctcgcccacgtggatgtggaggaagagggggcgggagcaaggggcgggcaccctcccttcaacctgacctgggacagtttcccttccgctcacctccgcctgagcagtggagaaggcggcactctggtggggctgctccaggcatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggccttgtggcctacatagccttcaagaggtggaacagtcatcgctatcctcgaggtcaccgcggtctagagtccacctgcagccaagcttatcgataatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggatccagatcccactgacgccctggccagtcgtctgggcggtgctacaactgggctggcggccaggatggttcttaggtaggtggggtcggcggtcaggtgtcccagagccaggggtctggagggaccttccaccctcagtccctggcaggtcggggggtgctgaggcgggcctggccctggcagcccaggggtcccggagcgaggggtctggagggacctttcactctcagtccctggcaggtcggggggtgctgtggcaggcccagccttggcccccagctctgccccttaccctgagctgtgtggctttgggcagctcgaactcctgggttcctctctgggccccaactcctcccctggcccaagtcccctctttgctcctgggcaggcaggacctctgtcccctctcagccggtccttggggctgcgtgtttctgtagaatgacgggtcaggctggccagaaccccaaaccttggccgtggggagtctgcgtggcggctcgcggccgc

SEQ ID NO: 23 - DNA, 2178 bp, Artificial Sequence
  Human NR4A2/NGFR HDR template
gcggccgccgaaaacgcctgtaactcggctgaaggttagtgcaacttcatttctttcctttactctccagagctccccaaacatcaagaaacaggacaaggcaaaccctgtaacttaaggtttgcccgacccatcgccttcgggaacaactttctcattgtgaaattcaacttcatttctagatggtcatttctagaaagagactgctgaatctgagcttcagagaagaggctcatctgagtgggatgagtgggggggtatgagggagatgtttggaaatacccaggagtgtagaccctcagtagctttttagctctgggtctttatttggttagtctttccacgccctaaactgttgttctgcagcattctctctctcctgcctttcctctcgcgcccctacatgctctctgactgccgcgggctgccggtgtagctccaggtgtacccgagcccgggagaaagtgttcagttgaccaggctgagtgtgttatcaccctgtttcatttccagccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggccttgtggcctacatagccttcaagaggtggaacagtcatcgctatcctcgaggtcaccgcggtctagagtccacctgcagccaagcttatcgataataatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtgctttgtgttcaggcgcagtatgggtcctcgcctcaaggagccagccccgcttctcagagctacagttaccactcttcgggagaatacagctccgatttcttaactccagagtttgtcaagtttagcatggacctcaccaacactgaaatcactgccaccacttctctccccagcttcagtacctttatggacaactacagcacaggctacgacgtcaagccaccttgcttgtaccaaatgcccctgtccggacagcagtcctccattaaggtagaagacattcagatgcacaactaccagcaacacagccacctgcccccccagtctgaggagatgatgccgcactccgggtcggtttactacaagccctcctcgcccccgacgcccaccaccccgggcttccaggtgcagcacagccccatgtgggacgacccgggatctctccacaacttccaccagaactacgtggccactacgcacatgatcgagcagaggaaaacgcgcggccgc

SEQ ID NO: 24 - DNA, 1681 bp, Artificial Sequence
  Human PD-1/CXCL9 HDR template
gcggccgctcactgcagcccaggaacctgagcccagagggggccacccaccttccccaggcagggaggcccggcccccagggagatgggggggatgggggaggagaagggcctgcccccacccggcagcctcaggaggggcagctcgggcgggatatggaaagaggccacagcagtgagcagagacacagaggaggaaggggccctgagctggggagacccccacggggtagggcgtgggggccacgggcccacctcctccccatctcctctgtctccctgtctctgtctctctctccctcccccaccctctccccagtcctaccccctcctcacccctcctcccccagcactgcctctgtcactctcgcccacgtggatgtggaggaagagggggcgggagcaaggggcgggcaccctcccttcaacctgacctgggacagtttcccttccgctcacctccgcctgagcagtggagaaggcggcactctggtggggctgctccaggcatgaagaaaagtggtgttcttttcctcttgggcatcatcttgctggttctgattggagtgcaaggaaccccagtagtgagaaagggtcgctgttcctgcatcagcaccaaccaagggactatccacctacaatccttgaaagaccttaaacaatttgccccaagcccttcctgcgagaaaattgaaatcattgctacactgaagaatggagttcaaacatgtctaaacccagattcagcagatgtgaaggaactgattaaaaagtgggagaaacaggtcagccaaaagaaaaagcaaaagaatgggaaaaaacatcaaaaaaagaaagttctgaaagttcgaaaatctcaacgttctcgtcaaaagaagactacataatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggatccagatcccactgacgccctggccagtcgtctgggcggtgctacaactgggctggcggccaggatggttcttaggtaggtggggtcggcggtcaggtgtcccagagccaggggtctggagggaccttccaccctcagtccctggcaggtcggggggtgctgaggcgggcctggccctggcagcccaggggtcccggagcgaggggtctggagggacctttcactctcagtccctggcaggtcggggggtgctgtggcaggcccagccttggcccccagctctgccccttaccctgagctgtgtggctttgggcagctcgaactcctgggttcctctctgggccccaactcctcccctggcccaagtcccctctttgctcctgggcaggcaggacctctgtcccctctcagccggtccttggggctgcgtgtttctgtagaatgacgggtcaggctggccagaaccccaaaccttggccgtggggagtctgcgtggcggctcgcggccgc

SEQ ID NO: 25 - DNA, 1689 bp, Artificial Sequence
  Human NR4A2/CXCL9 HDR template
gcggccgccgaaaacgcctgtaactcggctgaaggttagtgcaacttcatttctttcctttactctccagagctccccaaacatcaagaaacaggacaaggcaaaccctgtaacttaaggtttgcccgacccatcgccttcgggaacaactttctcattgtgaaattcaacttcatttctagatggtcatttctagaaagagactgctgaatctgagcttcagagaagaggctcatctgagtgggatgagtgggggggtatgagggagatgtttggaaatacccaggagtgtagaccctcagtagctttttagctctgggtctttatttggttagtctttccacgccctaaactgttgttctgcagcattctctctctcctgcctttcctctcgcgcccctacatgctctctgactgccgcgggctgccggtgtagctccaggtgtacccgagcccgggagaaagtgttcagttgaccaggctgagtgtgttatcaccctgtttcatttccagccatgaagaaaagtggtgttcttttcctcttgggcatcatcttgctggttctgattggagtgcaaggaaccccagtagtgagaaagggtcgctgttcctgcatcagcaccaaccaagggactatccacctacaatccttgaaagaccttaaacaatttgccccaagcccttcctgcgagaaaattgaaatcattgctacactgaagaatggagttcaaacatgtctaaacccagattcagcagatgtgaaggaactgattaaaaagtgggagaaacaggtcagccaaaagaaaaagcaaaagaatgggaaaaaacatcaaaaaaagaaagttctgaaagttcgaaaatctcaacgttctcgtcaaaagaagactacataatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtgctttgtgttcaggcgcagtatgggtcctcgcctcaaggagccagccccgcttctcagagctacagttaccactcttcgggagaatacagctccgatttcttaactccagagtttgtcaagtttagcatggacctcaccaacactgaaatcactgccaccacttctctccccagcttcagtacctttatggacaactacagcacaggctacgacgtcaagccaccttgcttgtaccaaatgcccctgtccggacagcagtcctccattaaggtagaagacattcagatgcacaactaccagcaacacagccacctgcccccccagtctgaggagatgatgccgcactccgggtcggtttactacaagccctcctcgcccccgacgcccaccaccccgggcttccaggtgcagcacagccccatgtgggacgacccgggatctctccacaacttccaccagaactacgtggccactacgcacatgatcgagcagaggaaaacgcgcggccgc

SEQ ID NO: 26 - DNA, 1600 bp, Artificial Sequence
  Human PD-1/CXCL10 HDR template
gcggccgctcactgcagcccaggaacctgagcccagagggggccacccaccttccccaggcagggaggcccggcccccagggagatgggggggatgggggaggagaagggcctgcccccacccggcagcctcaggaggggcagctcgggcgggatatggaaagaggccacagcagtgagcagagacacagaggaggaaggggccctgagctggggagacccccacggggtagggcgtgggggccacgggcccacctcctccccatctcctctgtctccctgtctctgtctctctctccctcccccaccctctccccagtcctaccccctcctcacccctcctcccccagcactgcctctgtcactctcgcccacgtggatgtggaggaagagggggcgggagcaaggggcgggcaccctcccttcaacctgacctgggacagtttcccttccgctcacctccgcctgagcagtggagaaggcggcactctggtggggctgctccaggcatgaatcaaactgccattctgatttgctgccttatctttctgactctaagtggcattcaaggagtacctctctctagaactgtacgctgtacctgcatcagcattagtaatcaacctgttaatccaaggtctttagaaaaacttgaaattattcctgcaagccaattttgtccacgtgttgagatcattgctacaatgaaaaagaagggtgagaagagatgtctgaatccagaatcgaaggccatcaagaatttactgaaagcagttagcaaggaaaggtctaaaagatctccttaatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggatccagatcccactgacgccctggccagtcgtctgggcggtgctacaactgggctggcggccaggatggttcttaggtaggtggggtcggcggtcaggtgtcccagagccaggggtctggagggaccttccaccctcagtccctggcaggtcggggggtgctgaggcgggcctggccctggcagcccaggggtcccggagcgaggggtctggagggacctttcactctcagtccctggcaggtcggggggtgctgtggcaggcccagccttggcccccagctctgccccttaccctgagctgtgtggctttgggcagctcgaactcctgggttcctctctgggccccaactcctcccctggcccaagtcccctctttgctcctgggcaggcaggacctctgtcccctctcagccggtccttggggctgcgtgtttctgtagaatgacgggtcaggctggccagaaccccaaaccttggccgtggggagtctgcgtggcggctcgcggccgc

SEQ ID NO: 27 - DNA, 1608 bp, Artificial Sequence
  Human NR4A2/CXCL10 HDR template
gcggccgccgaaaacgcctgtaactcggctgaaggttagtgcaacttcatttctttcctttactctccagagctccccaaacatcaagaaacaggacaaggcaaaccctgtaacttaaggtttgcccgacccatcgccttcgggaacaactttctcattgtgaaattcaacttcatttctagatggtcatttctagaaagagactgctgaatctgagcttcagagaagaggctcatctgagtgggatgagtgggggggtatgagggagatgtttggaaatacccaggagtgtagaccctcagtagctttttagctctgggtctttatttggttagtctttccacgccctaaactgttgttctgcagcattctctctctcctgcctttcctctcgcgcccctacatgctctctgactgccgcgggctgccggtgtagctccaggtgtacccgagcccgggagaaagtgttcagttgaccaggctgagtgtgttatcaccctgtttcatttccagccatgaatcaaactgccattctgatttgctgccttatctttctgactctaagtggcattcaaggagtacctctctctagaactgtacgctgtacctgcatcagcattagtaatcaacctgttaatccaaggtctttagaaaaacttgaaattattcctgcaagccaattttgtccacgtgttgagatcattgctacaatgaaaaagaagggtgagaagagatgtctgaatccagaatcgaaggccatcaagaatttactgaaagcagttagcaaggaaaggtctaaaagatctccttaatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtgctttgtgttcaggcgcagtatgggtcctcgcctcaaggagccagccccgcttctcagagctacagttaccactcttcgggagaatacagctccgatttcttaactccagagtttgtcaagtttagcatggacctcaccaacactgaaatcactgccaccacttctctccccagcttcagtacctttatggacaactacagcacaggctacgacgtcaagccaccttgcttgtaccaaatgcccctgtccggacagcagtcctccattaaggtagaagacattcagatgcacaactaccagcaacacagccacctgcccccccagtctgaggagatgatgccgcactccgggtcggtttactacaagccctcctcgcccccgacgcccaccaccccgggcttccaggtgcagcacagccccatgtgggacgacccgggatctctccacaacttccaccagaactacgtggccactacgcacatgatcgagcagaggaaaacgcgcggccgc

SEQ ID NO: 28 - DNA, 2041 bp, Artificial Sequence
  Human PD-1/CXCL9-P2A-CXCL10 HDR template
gcggccgctcactgcagcccaggaacctgagcccagagggggccacccaccttccccaggcagggaggcccggcccccagggagatgggggggatgggggaggagaagggcctgcccccacccggcagcctcaggaggggcagctcgggcgggatatggaaagaggccacagcagtgagcagagacacagaggaggaaggggccctgagctggggagacccccacggggtagggcgtgggggccacgggcccacctcctccccatctcctctgtctccctgtctctgtctctctctccctcccccaccctctccccagtcctaccccctcctcacccctcctcccccagcactgcctctgtcactctcgcccacgtggatgtggaggaagagggggcgggagcaaggggcgggcaccctcccttcaacctgacctgggacagtttcccttccgctcacctccgcctgagcagtggagaaggcggcactctggtggggctgctccaggcatgaagaaaagtggtgttcttttcctcttgggcatcatcttgctggttctgattggagtgcaaggaaccccagtagtgagaaagggtcgctgttcctgcatcagcaccaaccaagggactatccacctacaatccttgaaagaccttaaacaatttgccccaagcccttcctgcgagaaaattgaaatcattgctacactgaagaatggagttcaaacatgtctaaacccagattcagcagatgtgaaggaactgattaaaaagtgggagaaacaggtcagccaaaagaaaaagcaaaagaatgggaaaaaacatcaaaaaaagaaagttctgaaagttcgaaaatctcaacgttctcgtcaaaagaagactacaggatccggagctactaacttcagcctgctgaagcaggctggcgacgtggaggagaaccctggacctatgaatcaaactgccattctgatttgctgccttatctttctgactctaagtggcattcaaggagtacctctctctagaactgtacgctgtacctgcatcagcattagtaatcaacctgttaatccaaggtctttagaaaaacttgaaattattcctgcaagccaattttgtccacgtgttgagatcattgctacaatgaaaaagaagggtgagaagagatgtctgaatccagaatcgaaggccatcaagaatttactgaaagcagttagcaaggaaaggtctaaaagatctccttaatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggatccagatcccactgacgccctggccagtcgtctgggcggtgctacaactgggctggcggccaggatggttcttaggtaggtggggtcggcggtcaggtgtcccagagccaggggtctggagggaccttccaccctcagtccctggcaggtcggggggtgctgaggcgggcctggccctggcagcccaggggtcccggagcgaggggtctggagggacctttcactctcagtccctggcaggtcggggggtgctgtggcaggcccagccttggcccccagctctgccccttaccctgagctgtgtggctttgggcagctcgaactcctgggttcctctctgggccccaactcctcccctggcccaagtcccctctttgctcctgggcaggcaggacctctgtcccctctcagccggtccttggggctgcgtgtttctgtagaatgacgggtcaggctggccagaaccccaaaccttggccgtggggagtctgcgtggcggctcgcggccgc

SEQ ID NO: 29 - DNA, 2049 bp, Artificial Sequence
  Human NR4A2/CXCL9-P2A-CXCL10 HDR template
gcggccgccgaaaacgcctgtaactcggctgaaggttagtgcaacttcatttctttcctttactctccagagctccccaaacatcaagaaacaggacaaggcaaaccctgtaacttaaggtttgcccgacccatcgccttcgggaacaactttctcattgtgaaattcaacttcatttctagatggtcatttctagaaagagactgctgaatctgagcttcagagaagaggctcatctgagtgggatgagtgggggggtatgagggagatgtttggaaatacccaggagtgtagaccctcagtagctttttagctctgggtctttatttggttagtctttccacgccctaaactgttgttctgcagcattctctctctcctgcctttcctctcgcgcccctacatgctctctgactgccgcgggctgccggtgtagctccaggtgtacccgagcccgggagaaagtgttcagttgaccaggctgagtgtgttatcaccctgtttcatttccagccatgaagaaaagtggtgttcttttcctcttgggcatcatcttgctggttctgattggagtgcaaggaaccccagtagtgagaaagggtcgctgttcctgcatcagcaccaaccaagggactatccacctacaatccttgaaagaccttaaacaatttgccccaagcccttcctgcgagaaaattgaaatcattgctacactgaagaatggagttcaaacatgtctaaacccagattcagcagatgtgaaggaactgattaaaaagtgggagaaacaggtcagccaaaagaaaaagcaaaagaatgggaaaaaacatcaaaaaaagaaagttctgaaagttcgaaaatctcaacgttctcgtcaaaagaagactacaggatccggagctactaacttcagcctgctgaagcaggctggcgacgtggaggagaaccctggacctatgaatcaaactgccattctgatttgctgccttatctttctgactctaagtggcattcaaggagtacctctctctagaactgtacgctgtacctgcatcagcattagtaatcaacctgttaatccaaggtctttagaaaaacttgaaattattcctgcaagccaattttgtccacgtgttgagatcattgctacaatgaaaaagaagggtgagaagagatgtctgaatccagaatcgaaggccatcaagaatttactgaaagcagttagcaaggaaaggtctaaaagatctccttaatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtgctttgtgttcaggcgcagtatgggtcctcgcctcaaggagccagccccgcttctcagagctacagttaccactcttcgggagaatacagctccgatttcttaactccagagtttgtcaagtttagcatggacctcaccaacactgaaatcactgccaccacttctctccccagcttcagtacctttatggacaactacagcacaggctacgacgtcaagccaccttgcttgtaccaaatgcccctgtccggacagcagtcctccattaaggtagaagacattcagatgcacaactaccagcaacacagccacctgcccccccagtctgaggagatgatgccgcactccgggtcggtttactacaagccctcctcgcccccgacgcccaccaccccgggcttccaggtgcagcacagccccatgtgggacgacccgggatctctccacaacttccaccagaactacgtggccactacgcacatgatcgagcagaggaaaacgcgcggccgc

SEQ ID NO: 30 - DNA, 2005 bp, Artificial Sequence
  Human PD-1/TNF HDR template
gcggccgctcactgcagcccaggaacctgagcccagagggggccacccaccttccccaggcagggaggcccggcccccagggagatgggggggatgggggaggagaagggcctgcccccacccggcagcctcaggaggggcagctcgggcgggatatggaaagaggccacagcagtgagcagagacacagaggaggaaggggccctgagctggggagacccccacggggtagggcgtgggggccacgggcccacctcctccccatctcctctgtctccctgtctctgtctctctctccctcccccaccctctccccagtcctaccccctcctcacccctcctcccccagcactgcctctgtcactctcgcccacgtggatgtggaggaagagggggcgggagcaaggggcgggcaccctcccttcaacctgacctgggacagtttcccttccgctcacctccgcctgagcagtggagaaggcggcactctggtggggctgctccaggcatgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtgatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggatccagatcccactgacgccctggccagtcgtctgggcggtgctacaactgggctggcggccaggatggttcttaggtaggtggggtcggcggtcaggtgtcccagagccaggggtctggagggaccttccaccctcagtccctggcaggtcggggggtgctgaggcgggcctggccctggcagcccaggggtcccggagcgaggggtctggagggacctttcactctcagtccctggcaggtcggggggtgctgtggcaggcccagccttggcccccagctctgccccttaccctgagctgtgtggctttgggcagctcgaactcctgggttcctctctgggccccaactcctcccctggcccaagtcccctctttgctcctgggcaggcaggacctctgtcccctctcagccggtccttggggctgcgtgtttctgtagaatgacgggtcaggctggccagaaccccaaaccttggccgtggggagtctgcgtggcggctcgcggccgc

SEQ ID NO: 31 - DNA, 2013 bp, Artificial Sequence
  Human NR4A2/TNF HDR template
gcggccgccgaaaacgcctgtaactcggctgaaggttagtgcaacttcatttctttcctttactctccagagctccccaaacatcaagaaacaggacaaggcaaaccctgtaacttaaggtttgcccgacccatcgccttcgggaacaactttctcattgtgaaattcaacttcatttctagatggtcatttctagaaagagactgctgaatctgagcttcagagaagaggctcatctgagtgggatgagtgggggggtatgagggagatgtttggaaatacccaggagtgtagaccctcagtagctttttagctctgggtctttatttggttagtctttccacgccctaaactgttgttctgcagcattctctctctcctgcctttcctctcgcgcccctacatgctctctgactgccgcgggctgccggtgtagctccaggtgtacccgagcccgggagaaagtgttcagttgaccaggctgagtgtgttatcaccctgtttcatttccagccatgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtgatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtgctttgtgttcaggcgcagtatgggtcctcgcctcaaggagccagccccgcttctcagagctacagttaccactcttcgggagaatacagctccgatttcttaactccagagtttgtcaagtttagcatggacctcaccaacactgaaatcactgccaccacttctctccccagcttcagtacctttatggacaactacagcacaggctacgacgtcaagccaccttgcttgtaccaaatgcccctgtccggacagcagtcctccattaaggtagaagacattcagatgcacaactaccagcaacacagccacctgcccccccagtctgaggagatgatgccgcactccgggtcggtttactacaagccctcctcgcccccgacgcccaccaccccgggcttccaggtgcagcacagccccatgtgggacgacccgggatctctccacaacttccaccagaactacgtggccactacgcacatgatcgagcagaggaaaacgcgcggccgc

SEQ ID NO: 32 - DNA, 1804 bp, Artificial Sequence
  Human PD-1/IFNg HDR template
gcggccgctcactgcagcccaggaacctgagcccagagggggccacccaccttccccaggcagggaggcccggcccccagggagatgggggggatgggggaggagaagggcctgcccccacccggcagcctcaggaggggcagctcgggcgggatatggaaagaggccacagcagtgagcagagacacagaggaggaaggggccctgagctggggagacccccacggggtagggcgtgggggccacgggcccacctcctccccatctcctctgtctccctgtctctgtctctctctccctcccccaccctctccccagtcctaccccctcctcacccctcctcccccagcactgcctctgtcactctcgcccacgtggatgtggaggaagagggggcgggagcaaggggcgggcaccctcccttcaacctgacctgggacagtttcccttccgctcacctccgcctgagcagtggagaaggcggcactctggtggggctgctccaggcatgaaatatacaagttatatcttggcttttcagctctgcatcgttttgggttctcttggctgttactgccaggacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccagtaatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggatccagatcccactgacgccctggccagtcgtctgggcggtgctacaactgggctggcggccaggatggttcttaggtaggtggggtcggcggtcaggtgtcccagagccaggggtctggagggaccttccaccctcagtccctggcaggtcggggggtgctgaggcgggcctggccctggcagcccaggggtcccggagcgaggggtctggagggacctttcactctcagtccctggcaggtcggggggtgctgtggcaggcccagccttggcccccagctctgccccttaccctgagctgtgtggctttgggcagctcgaactcctgggttcctctctgggccccaactcctcccctggcccaagtcccctctttgctcctgggcaggcaggacctctgtcccctctcagccggtccttggggctgcgtgtttctgtagaatgacgggtcaggctggccagaaccccaaaccttggccgtggggagtctgcgtggcggctcgcggccgc

SEQ ID NO: 33 - DNA, 1812 bp, Artificial Sequence
  Human NR4A2/IFNg HDR template
gcggccgccgaaaacgcctgtaactcggctgaaggttagtgcaacttcatttctttcctttactctccagagctccccaaacatcaagaaacaggacaaggcaaaccctgtaacttaaggtttgcccgacccatcgccttcgggaacaactttctcattgtgaaattcaacttcatttctagatggtcatttctagaaagagactgctgaatctgagcttcagagaagaggctcatctgagtgggatgagtgggggggtatgagggagatgtttggaaatacccaggagtgtagaccctcagtagctttttagctctgggtctttatttggttagtctttccacgccctaaactgttgttctgcagcattctctctctcctgcctttcctctcgcgcccctacatgctctctgactgccgcgggctgccggtgtagctccaggtgtacccgagcccgggagaaagtgttcagttgaccaggctgagtgtgttatcaccctgtttcatttccagccatgaaatatacaagttatatcttggcttttcagctctgcatcgttttgggttctcttggctgttactgccaggacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccagtaatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtgctttgtgttcaggcgcagtatgggtcctcgcctcaaggagccagccccgcttctcagagctacagttaccactcttcgggagaatacagctccgatttcttaactccagagtttgtcaagtttagcatggacctcaccaacactgaaatcactgccaccacttctctccccagcttcagtacctttatggacaactacagcacaggctacgacgtcaagccaccttgcttgtaccaaatgcccctgtccggacagcagtcctccattaaggtagaagacattcagatgcacaactaccagcaacacagccacctgcccccccagtctgaggagatgatgccgcactccgggtcggtttactacaagccctcctcgcccccgacgcccaccaccccgggcttccaggtgcagcacagccccatgtgggacgacccgggatctctccacaacttccaccagaactacgtggccactacgcacatgatcgagcagaggaaaacgcgcggccgc

SEQ ID NO: 34 - DNA, 2569 bp, Artificial Sequence
  Human PD-1/TNF-P2A-IFNg HDR template
gcggccgctcactgcagcccaggaacctgagcccagagggggccacccaccttccccaggcagggaggcccggcccccagggagatgggggggatgggggaggagaagggcctgcccccacccggcagcctcaggaggggcagctcgggcgggatatggaaagaggccacagcagtgagcagagacacagaggaggaaggggccctgagctggggagacccccacggggtagggcgtgggggccacgggcccacctcctccccatctcctctgtctccctgtctctgtctctctctccctcccccaccctctccccagtcctaccccctcctcacccctcctcccccagcactgcctctgtcactctcgcccacgtggatgtggaggaagagggggcgggagcaaggggcgggcaccctcccttcaacctgacctgggacagtttcccttccgctcacctccgcctgagcagtggagaaggcggcactctggtggggctgctccaggcatgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgggatccggagctactaacttcagcctgctgaagcaggctggcgacgtggaggagaaccctggacctatgaaatatacaagttatatcttggcttttcagctctgcatcgttttgggttctcttggctgttactgccaggacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccagtaatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggatccagatcccactgacgccctggccagtcgtctgggcggtgctacaactgggctggcggccaggatggttcttaggtaggtggggtcggcggtcaggtgtcccagagccaggggtctggagggaccttccaccctcagtccctggcaggtcggggggtgctgaggcgggcctggccctggcagcccaggggtcccggagcgaggggtctggagggacctttcactctcagtccctggcaggtcggggggtgctgtggcaggcccagccttggcccccagctctgccccttaccctgagctgtgtggctttgggcagctcgaactcctgggttcctctctgggccccaactcctcccctggcccaagtcccctctttgctcctgggcaggcaggacctctgtcccctctcagccggtccttggggctgcgtgtttctgtagaatgacgggtcaggctggccagaaccccaaaccttggccgtggggagtctgcgtggcggctcgcggccgc

SEQ ID NO: 35 - DNA, 2577 bp, Artificial Sequence
  Human NR4A2/TNF-P2A-IFNg HDR template
gcggccgccgaaaacgcctgtaactcggctgaaggttagtgcaacttcatttctttcctttactctccagagctccccaaacatcaagaaacaggacaaggcaaaccctgtaacttaaggtttgcccgacccatcgccttcgggaacaactttctcattgtgaaattcaacttcatttctagatggtcatttctagaaagagactgctgaatctgagcttcagagaagaggctcatctgagtgggatgagtgggggggtatgagggagatgtttggaaatacccaggagtgtagaccctcagtagctttttagctctgggtctttatttggttagtctttccacgccctaaactgttgttctgcagcattctctctctcctgcctttcctctcgcgcccctacatgctctctgactgccgcgggctgccggtgtagctccaggtgtacccgagcccgggagaaagtgttcagttgaccaggctgagtgtgttatcaccctgtttcatttccagccatgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgggatccggagctactaacttcagcctgctgaagcaggctggcgacgtggaggagaaccctggacctatgaaatatacaagttatatcttggcttttcagctctgcatcgttttgggttctcttggctgttactgccaggacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccagtaatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtgctttgtgttcaggcgcagtatgggtcctcgcctcaaggagccagccccgcttctcagagctacagttaccactcttcgggagaatacagctccgatttcttaactccagagtttgtcaagtttagcatggacctcaccaacactgaaatcactgccaccacttctctccccagcttcagtacctttatggacaactacagcacaggctacgacgtcaagccaccttgcttgtaccaaatgcccctgtccggacagcagtcctccattaaggtagaagacattcagatgcacaactaccagcaacacagccacctgcccccccagtctgaggagatgatgccgcactccgggtcggtttactacaagccctcctcgcccccgacgcccaccaccccgggcttccaggtgcagcacagccccatgtgggacgacccgggatctctccacaacttccaccagaactacgtggccactacgcacatgatcgagcagaggaaaacgcgcggccgc

SEQ ID NO: 36 - DNA, 864 bp, Artificial Sequence
  Human NGFR cDNA
atggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggccttgtggcctacatagccttcaagaggtggaacagtcatcgctatcctcgaggtcaccgcggtctagagtccacctgcagccaagcttatcgataa

SEQ ID NO: 37 - DNA, 378 bp, Artificial Sequence
  Human CXCL9 cDNA
atgaagaaaagtggtgttcttttcctcttgggcatcatcttgctggttctgattggagtgcaaggaaccccagtagtgagaaagggtcgctgttcctgcatcagcaccaaccaagggactatccacctacaatccttgaaagaccttaaacaatttgccccaagcccttcctgcgagaaaattgaaatcattgctacactgaagaatggagttcaaacatgtctaaacccagattcagcagatgtgaaggaactgattaaaaagtgggagaaacaggtcagccaaaagaaaaagcaaaagaatgggaaaaaacatcaaaaaaagaaagttctgaaagttcgaaaatctcaacgttctcgtcaaaagaagactacataa

SEQ ID NO: 38 - DNA, 297 bp, Artificial Sequence
  Human CXCL10 cDNA
atgaatcaaactgccattctgatttgctgccttatctttctgactctaagtggcattcaaggagtacctctctctagaactgtacgctgtacctgcatcagcattagtaatcaacctgttaatccaaggtctttagaaaaacttgaaattattcctgcaagccaattttgtccacgtgttgagatcattgctacaatgaaaaagaagggtgagaagagatgtctgaatccagaatcgaaggccatcaagaatttactgaaagcagttagcaaggaaaggtctaaaagatctccttaa

SEQ ID NO: 39 - DNA, 738 bp, Artificial Sequence
  Human CXCL9-P2A-CXCL10 cDNA
atgaagaaaagtggtgttcttttcctcttgggcatcatcttgctggttctgattggagtgcaaggaaccccagtagtgagaaagggtcgctgttcctgcatcagcaccaaccaagggactatccacctacaatccttgaaagaccttaaacaatttgccccaagcccttcctgcgagaaaattgaaatcattgctacactgaagaatggagttcaaacatgtctaaacccagattcagcagatgtgaaggaactgattaaaaagtgggagaaacaggtcagccaaaagaaaaagcaaaagaatgggaaaaaacatcaaaaaaagaaagttctgaaagttcgaaaatctcaacgttctcgtcaaaagaagactacaggatccggagctactaacttcagcctgctgaagcaggctggcgacgtggaggagaaccctggacctatgaatcaaactgccattctgatttgctgccttatctttctgactctaagtggcattcaaggagtacctctctctagaactgtacgctgtacctgcatcagcattagtaatcaacctgttaatccaaggtctttagaaaaacttgaaattattcctgcaagccaattttgtccacgtgttgagatcattgctacaatgaaaaagaagggtgagaagagatgtctgaatccagaatcgaaggccatcaagaatttactgaaagcagttagcaaggaaaggtctaaaagatctccttaa

SEQ ID NO: 40 - DNA, 702 bp, Artificial Sequence
  Human TNF cDNA
atgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtga

SEQ ID NO: 41 - DNA, 501 bp, Artificial Sequence
  Human IFNg cDNA
atgaaatatacaagttatatcttggcttttcagctctgcatcgttttgggttctcttggctgttactgccaggacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccagtaa

SEQ ID NO: 42 - DNA, 1266 bp, Artificial Sequence
  Human TNF-P2A-IFNg cDNA
atgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgggatccggagctactaacttcagcctgctgaagcaggctggcgacgtggaggagaaccctggacctatgaaatatacaagttatatcttggcttttcagctctgcatcgttttgggttctcttggctgttactgccaggacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccagtaa

SEQ ID NO: 43 - DNA, 63 bp, Artificial Sequence
  P2A linker
tccggagctactaacttcagcctgctgaagcaggctggcgacgtggaggagaaccctggacct

SEQ ID NO: 44 - DNA, 63 bp, Artificial Sequence
  T2A linker
ggctccggcgagggcaggggaagtcttctaacatgcggggacgtggaggaaaatcccggccca

SEQ ID NO: 45 - DNA, 284 bp, Artificial Sequence
  PolyA tail
tctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 46 - DNA, 3 bp, Artificial Sequence
  PAM
  misc_feature (1)..(1): n is a, c, g, or t
ngg

(19) United States
(12) Patent Application Publication
(10) Pub. No.: US 2025/0249096 A1
(43) Pub. Date: Aug. 7, 2025

(54) Compositions and Methods for Immunotherapy

(71) Applicant: Peter MacCallum Cancer Institute, Melbourne, Victoria (AU)

(72) Inventors: Paul Beavis, Reservoir, Victoria (AU); Phillip Kevin Darcy, Yallambie, Victoria (AU); Amanda Xi Ying Chen, Melbourne, Victoria (AU); Imran Geoffrey House, Melbourne, Victoria (AU)

(21) Appl. No.: 18/702,293

(22) PCT Filed: Oct. 19, 2021
(86) PCT No.: PCT/AU2021/051219

§ 371 Date: Apr. 17, 2024

(51) Int. Cl. A61K 40/11; A61K 40/31; A61K 40/42; A61P 35/00; C12N 5/0783; C12N 9/22; C12N 15/11; C12N 15/864

(52) U.S. Cl. CPC A61K 40/11; A61K 40/31; A61K 40/4205; A61P 35/00; C12N 5/0636; C12N 9/22; C12N 15/11; C12N 15/8645; C12N 2310/20; C12N 2510/00; C12N 2750/14141

(57) ABSTRACT

The present disclosure relates generally to T cells, e.g., CAR T cells, which have been engineered to express immunomodulatory factors in a tumor-site specific manner. The engineered T cells and pharmaceutical compositions comprising the engineered T cells exhibit improved therapeutic efficacy and reduced toxicity when used for the treatment of cancer. In other embodiments contemplated herein, the present disclosure relates to genome editing systems for engineering T cells to express immunomodulatory factors in a tumor-site specific manner.

### FIELD

The present disclosure relates generally to T cells, e.g., CAR T cells, which have been engineered to express immunomodulatory factors in a tumor-site specific manner. The engineered T cells and pharmaceutical compositions comprising the engineered T cells exhibit improved therapeutic efficacy and reduced toxicity when used for the treatment of cancer. In other embodiments contemplated herein, the present disclosure relates to genome editing systems for engineering T cells to express immunomodulatory factors in a tumor-site specific manner.

### BACKGROUND

Chimeric antigen receptor (CAR) T cell therapy has proven to be highly efficacious in the treatment of hematological malignancies, with a number of FDA approved CAR T cell products currently available, including Kymriah (tisagenlecleucel), Yescarta (axicabtagene ciloleucel), Tecartus (brexucabtagene autolecucel), Abecma (idecabtagene vicleucel) and Breyanzi (lisocabtagene maraleucel). Each of these products target CD19, and have been shown to be effective for the treatment of B cell acute lymphoblastic leukaemia (B-ALL), chronic lymphocytic leukaemia (CLL) and non-Hodgkin's lymphoma (NHL) (Grupp et al., 2013, New England Journal of Medicine, 368: 1509-1518; Maude et al., 2018, The New England Journal of Medicine, 378(5): 439-448; Park et al., 2018, The New England Journal of Medicine, 378(5): 449-459; Porter et al., 2011, The New England Journal of Medicine, 365: 725-733).

While CAR T cells have demonstrated efficacy for the treatment of haematological malignancies, this is yet to be achieved for the treatment of solid cancers. In the context of solid tumors, CAR T cells face various barriers that limit their therapeutic efficacy, including tumor heterogeneity, inefficient trafficking into the tumor, and immunosuppressive mechanisms in the tumor microenvironment. Engineering CAR T cells to express immunomodulatory factors, such as pro-inflammatory cytokines, is a strategy that has the potential to overcome multiple limitations of CAR T cell therapy by eliciting widespread effects on endogenous immune mechanisms. While these so-called “armored” CAR T cells have demonstrated promising results in pre-clinical models, their clinical application been limited by severe, life-threatening toxicities including liver dysfunction, high fevers and hemodynamic instability, which may be attributable to constitutive, uncontrolled or unintended transgene expression (Zhang et al., 2015, Clinical Cancer Research, 21: 2278-2288).

Accordingly, there remains a need for the development of improved engineered T cells, and methods for their production, which enable their safe and effective use for immunotherapy, e.g., for the treatment of cancer.

### SUMMARY

In an aspect of the present disclosure, there is provided an engineered T cell comprising a heterologous nucleotide sequence encoding at least one immunomodulatory factor, wherein the heterologous nucleotide sequence is introduced in-frame of an endogenous gene of the T cell encoding a tumor-specific factor under the control of endogenous regulatory elements, wherein expression of the heterologous nucleotide sequence is controlled by the endogenous regulatory elements of the endogenous gene.

In another aspect of the present disclosure, there is provided a pharmaceutical composition comprising the engineered T cell (i.e., a population of the engineered T cells) described herein.

In another aspect of the present disclosure, there is provided a method for the treatment of cancer comprising the administration of a therapeutically effective amount of the engineered T cell or composition described herein to a subject in need thereof.

In another aspect of the present disclosure, there is provided a use of the engineered T cell or composition described herein in the manufacture of a medicament for the treatment of cancer.

In another aspect of the present disclosure, there is provided a genome editing system comprising:
(a) a sgRNA comprising a sequence of at least 10 contiguous nucleotides that are complementary to a target nucleic acid sequence within an endogenous gene of the T cell encoding a tumor-specific factor under the control of endogenous regulatory elements;
(b) a RNA-guided nuclease; and
(c) a homology directed repair (HDR) template, wherein the HDR template comprises a nucleotide sequence encoding at least one immunomodulatory factor.

In another aspect of the present disclosure, there is provided a method of altering a nucleic acid molecule in a T cell, the method comprising providing to the T cell the genome editing system as described herein.

### BRIEF DESCRIPTION OF THE DRAWINGS

Embodiments of the disclosure are described herein, by way of non-limiting example only, with reference to the accompanying drawings.

FIG. 1 shows that use of a nuclear factor of T cells (NFAT)-responsive element to drive GFP expression leads to CAR antigen induced expression of GFP in vitro. (A) A schematic representation of a retroviral vector for NFAT inducible GFP expression. (B) A series of graphical representations of CD69 (y-axis) and GFP (x-axis) expression in non-stimulated (left panels), anti-CD3/anti-CD28 stimulated (middle panels), or tumor stimulated (right panels) CAR T cells. (C) A graphical representation of GFP expression (MFI; Comp-GFP-A::NFAT) in stimulated or unstimulated CAR T cells (y-axis). uTAG refers to an anti-Tag antibody stimulation that activates the anti-Her2 CAR directly. Numbers indicative of mean fluorescence intensity (MFI).

FIG. 2 shows that use of an NFAT-responsive element to drive GFP expression fails to induce tumor site-specific expression of GFP in vivo. (A) Representative FACSs plots of of NGFR (y-axis) and GFP (x-axis) expression by CD8+ CAR T cells post-adoptive transfer in tumors (top panels) and spleens (bottom panels). (B) A graphical representation of GFP MFI in CD8+ NGFR+ CAR T cells isolated from tumors and spleens. (C) A graphical representation of GFP expression (y-axis; GFP MFI) by CD8+ NGFR+ CAR T cells isolated from tumors and spleens (x-axis), shown as mean±SEM of n=6 mice per group. ****p<0.0001, ***p<0.001, **p<0.01, *p<0.05, one-way ANOVA.

FIG. 3 shows that a comparison of the transcriptome of murine CD8+ anti-Her2 CAR T cells isolated from the tumors (CD62L−) and spleens (CD62L+) of treated mice identifies genes selectively expressed in the tumor. (A) A graphical representation of fold change (y-axis; log 2(FC)) and mean expression (x-axis) in CD62L− CAR T cells isolated from the tumor site relative to CD62L+ CAR T cells isolated from the spleen. (B) A series of graphical representations of gene expression (y-axis, normalized counts per million (CPM)) for genes with increased expression in CD62L⁻ or CD62L⁺ CAR T cells isolated from the tumor site relative to CD62L⁺ CAR T cells isolated from the spleen. (C) A series of graphical representations of expression (y-axis, normalized CPM) for genes with increased expression in CD62L⁺ cells isolated from the spleen relative to CD62L− or CD62L+ CAR T cells isolated from the tumor site (x-axis).

FIG. 4 shows the design of DNA templates used to insert heterologous sequences into endogenous genes of T cells encoding tumor-specific factors. Schematic representations of the DNA templates used to insert GFP into (A) Pdcd1 (i.e., murine PD-1) and (B) NRFA2. “ATG” represents translation start site of the endogenous gene, “*” represents a stop codon, “pA” refers to a polyA tail, “PAM” refers to the protospacer adjacent motif. “HA” refers to the homology arms of the HDR template.

FIG. 5 shows HDR editing efficiency using polymerase chain reaction (PCR)-generated double stranded DNA (dsDNA) repair templates in primary T cells. (A) A series of graphical representations of GFP (y-axis) and PD-1 (x-axis) expression in CD8+ T cells isolated from OT-I spleens, which were electroporated with a Cas9/PD-1 sgRNA ribonucleoprotein (RNP) and PD-1/GFP HDR template (i.e., CRISPR-HDR edited OT-I T cells). (B) A series of graphical representations of GFP (y-axis) and PD-1 (x-axis) expression in CD4⁺ and CD8⁺ T cells derived from activated human peripheral blood mononuclear cells (PBMCs), which were electroporated with a Cas9/PD-1 sgRNA RNP and PD-1/GFP HDR template (i.e., CRISPR-HDR edited PBMCs).

FIG. 6 shows that transduction efficiency varies between AAV serotypes in primary T cells. (A) A graphical representation of transduction efficiency (y-axis; % GFP positive) and AAV serotype (x-axis) in murine C57BL/6 splenocytes activated with soluble anti-CD3/anti-CD28, TL-2 and TL-7. (B) A graphical representation of transduction efficiency (y-axis; % GFP positive) and AAV serotype (x-axis) in human PBMCs activated with anti-CD3 and IL-2.

FIG. 7 shows that AAV5- and AAV6-mediated delivery of HDR template is effective for editing T cells. A series of graphical representations of GFP (y-axis) and PD-1 (x-axis) expression in CRISPR-HDR edited OT-I cells transduced with AAV5 (middle panels) or AAV6 (right panels) encoding the PD-1/GFP HDR repair template.

FIG. 8 shows that optimization of MOI and infection volume improves HDR editing efficiency. (A) Representative FACS plots showing GFP (y-axis) and PD-1 (x-axis) expression in electroporated OT-I cells transduced with AAV6 at an MOI of 0; 50,000; 100,000; 200,000; 400,000 and 800,000. (B) Representative FACS plots showing GFP (y-axis) and PD-1 (x-axis) expression in electroporated OT-I cells transduced with 3.2; 1.6; 0.8; 0.4 and 0.2 vg/μL AAV6 at an MOI of 200,000. (C) Representative FACS plots showing GFP (y-axis) and PD-1 (x-axis) expression in electroporated OT-I cells incubated for 4; 16; and 72 hours with AAV6 at an MOI of 200,000.

FIG. 9 shows that inhibition of non-homologous end joining (NHEJ) improves HDR editing efficiency. (A) Representative FACS plots showing GFP (y-axis) and PD-1 (x-axis) expression in CRISPR-HDR edited OT-I cells pre-treated with 2 μM M3814 prior to electroporation. (B) Representative FACS plots showing GFP (y-axis) and PD-1 (x-axis) expression in CRISPR-HDR edited OT-I cells incubated with a range of M3814 concentrations, without pre-treatment. (C) Representative FACS plots GFP (y-axis) and PD-1 (x-axis) expression in CRISPR-HDR edited OT-I cells incubated with the indicated MOIs of PD-1/GFP AAV6 for 4 h with or without 2 μM M3814. (D) Representative FACS plots of CRISPR-HDR edited OT-I cells incubated with 2 μM M3814 for 4, 24 or 72 h, during transduction or both during and after transduction.

FIG. 10 shows that CRISPR-HDR editing of T cells at the PD-1 or NR4A2 target loci enhances tumor-stimulated GFP expression in vitro. (A) Representative FACS plots showing GFP (y-axis) and PD-1 (x-axis) expression in CRISPR-HDR edited OT-I cells co-cultured with MC38 or MC38 ova colon adenocarcinoma tumor cell lines. (B) A graphical representation of GFP expression (y-axis; % GFP+) by CRISPR-HDR edited OT-I cells co-cultured with a range of matched chicken ovalbumin (ova)⁺ and parental tumor cell lines (x-axis). Data shown as mean±SD of triplicate cultures, n=3. ****p<0.0001, one-way ANOVA. (C) A graphical representation of GFP induction (y-axis; average fluorescence) over time (x-axis; hours) in CRISPR-HDR edited OT-I cells co-cultured with the E0771 ova or E0771 parental breast tumor cell line as determined by Incucyte assay. Data presented as mean±SD of triplicate cultures, n=3. ****p<0.0001, two-way ANOVA.

FIG. 11 shows that CRISPR-HDR editing of T cells at the PD-1 or NR4A2 target loci results in tumor-site specific expression of GFP in vivo. (A) Representative FACS plots showing GFP (y-axis) and PD-1 (x-axis) expression in CRISPR-HDR edited CD45+CD8+α2+ cells isolated from tumors and spleens of tumor-bearing mice treated with CRISPR-HDR edited OT-I T cells. (B) A graphical representation of GFP expression (y-axis; % GFP+) and target loci/HDR template (x-axis). Data presented as mean±SEM, n=4 mice per group.

FIG. 12 shows that CRISPR-HDR editing of CAR T cells at the PD-1 or NR4A2 target loci results in activation-induced upregulation of GFP in vitro and in vivo. (A) Representative FACS plots showing anti-Her2 CAR (y-axis; anti-Myc tag) and FSC-A (x-axis) expression in CRISPR-HDR edited C57BL/6 splenocytes transduced with an anti-Her2 CAR (i.e., CRISPR-HDR edited CAR T cells). (B) Representative FACS plots showing GFP (y-axis) and PD-1 (x-axis) expression by CRISPR-HDR edited CD8+ T cells or CRISPR-HDR edited CD8+ CAR T cells. (C) Representative FACS plots showing GFP (y-axis) and PD-1 (x-axis) expression by CRISPR-HDR edited CAR T cells following co-culture with a Her2+ tumor cell line (E0771-Her2) or the Her2 negative parental line (E0771 parental). (D) A graphical representation of GFP expression (y-axis; % GFP+) by CRISPR-HDR edited CAR T cells co-cultured with a range of tumor cell lines (x-axis). Data shown as mean±SD of triplicate cultures, n=2. (E) Representative FACS plots showing GFP (y-axis) and PD-1 or CD62L (x-axis) expression by CRISPR-HDR edited CAR T cells (CD8+NGFR+ cells) isolated from tumors and spleens of mice bearing E0771-Her2 tumors.

FIG. 13 shows that CRISPR-HDR editing of T cells to integrate TNF into at the PD-1 or NR4A2 target loci enhances TNF expression. (A) A series of graphical representations of intracellular IFNγ and TNF expression (y-axis; percent IFNγ+, TNF+ or IFNγ+TNF+) by CAR T cells (CD8+NGFR+CAR T cells) isolated from tumor bearing mice post-treatment (x-axis; days post treatment). (B) Representative FACS plots of TNF (y-axis) and PD-1 (x-axis) expression by CRISPR-HDR edited OT-I T cells co-cultured with MC38 and MC38ova tumor cell lines. (C) A graphical representation of TNF expression (y-axis; percent TNF+) by CRISPR-HDR edited OT-I T cells co-culture with a range of ova+ and parental tumor cell lines (x-axis). (D) A graphical representation of TNF concentration (y-axis; TNF ng/mL) in the supernatant of CRISPR-HDR edited OT-I T cells co-cultured with a range of ova+ and parental tumor cell lines at 24 hours (top panels) and 72 hours (bottom panels).

FIG. 14 shows that CRISPR-HDR editing of T cells to integrate TNF into the PD-1 or NR4A2 target loci enhances cytotoxicity. (A) A series of graphical representations of cytotoxicity (y-axis; percent killing) and effector to target ratio (x-axis) of CRISPR-HDR edited OT-I T cells co-cultured with MC38ova and MC38 tumor cells. (B) A graphical representation of cytotoxicity (y-axis; percent killing) and supernatant dilution factor (x-axis) of MC38 tumor cells incubated with the supernatants from co-cultures of CRISPR-HDR edited OT-I T cells and MC38ova tumor cells taken at 24 hours (top panels) and 72 hours (bottom panels). (C) A graphical representation of the number of live tumor cells (y-axis) over time (x-axis; hours) in co-cultures of CRISPR-HDR edited OT-I T cells and MC38ova or MC38 tumor mCherry control tumor cells as quantified by the Incucyte assay, as described elsewhere herein. Images were taken every 4 hours. Data presented as mean±SD of triplicate cultures. ****p<0.0001, two-way ANOVA.

FIG. 15 shows that CRISPR-HDR editing of CAR T cells to integrate TNF into the PD-1 or NR4A2 target loci enhances TNF expression in vitro. (A) A series of graphical representations of intracellular TNF expression (y-axis) and target loci/HDR template (x-axis) following 72 hours stimulation with an anti-myc Tag. (B) A series of graphical representations of intracellular TNF expression (y-axis) and target loci/HDR template (x-axis) following 72 hours stimulation with indicated Her2 expressing tumor lines. (C) A series of graphical representations of TNF protein (pg/ml) in supernatants (y-axis) and target loci/HDR template (x-axis) following 72 hours stimulation with indicated Her2 expressing tumor lines or anti-myc Tag. ****p<0.0001, ***p<0.001, **p<0.01, *p<0.05 one way ANOVA.

FIG. 16 shows that CRISPR-HDR editing of T cells to integrate CXCL9 into the PD-1 or NR4A2 target loci enhances CXCL9 expression in vitro. (A) Representative FACS plots showing CXCL9 (y-axis) and PD-1 (x-axis) expression in CRISPR-HDR edited OT-I T cells following co-culture with MC38ova or MC38 tumor cells. (B) A graphical representation of CXCL9 expression (y-axis; percent CXCL9+) by CRISPR-HDR edited OT-I T cells following co-culture with tumor cell lines (x-axis). Data shown as mean±SD of triplicate cultures, n=3. ****p<0.0001, one-way ANOVA. (C) A graphical representation of CXCL9 protein (y-axis; pg/ml CXCL9) secreted by CRISPR-HDR edited OT-I T cells following stimulation with anti-CD3/anti-CD28. Data shown as mean±SD of triplicate cultures, n=3. ****p<0.0001, one-way ANOVA.

FIG. 17 shows that CRISPR-HDR editing of T cells to integrate CXCL9 at the PD-1 or NR4A2 target loci enhances CXCL9 expression in vivo. (A) Representative FACS plots showing CXCL9 (y-axis) and PD-1 (x-axis) expression by CRISPR-HDR edited OT-I T cells isolated from tumors and spleen of tumor-bearing Ly5/1 mice. (B) A graphical representation of CXCL9 expression (y-axis; % CXCL9+) by CRISPR-HDR edited OT-I T cells isolated from tumors and spleen of tumor-bearing Ly5/1 mice as compared to mock edited or PD-1 KO OT-I T cells (x-axis). (C) A graphical representation of CXCL9 expression (y-axis; % CXCL9+) by CRISPR-HDR edited OT-I T cells isolated from tumors and spleen of tumor-bearing Ly5/1 mice as compared to mock edited or NR4A2-KO OT-I T cells (x-axis). Data represented as mean±SEM of 5-6 mice per group.

FIG. 18 shows that CRISPR-HDR editing of T cells to integrate TNF into the PD-1 target locus enhances therapeutic activity. (A) A graphical representation of AT3ova tumor size (y-axis, mm²) following treatment with CRISPR-HDR or CRISPR/KO edited OT-I T cells (x-axis; days post treatment). (B) A graphical representation of MC38ova tumor size (y-axis, mm²) following treatment with CRISPR-HDR or CRISPR/KO edited OT-I T cells (x-axis; days post treatment). Tumor sizes are presented as mean±SEM of 4-6 mice per group. ***p<0.001, **p<0.01, two-way ANOVA. (C) A photographic representation of tumor size from AT3ova tumors excised 8 days after treatment with CRISPR-HDR or CRISPR/KO edited OT-I T cells.

FIG. 19 shows that a comparison of the transcriptome of human anti-Lewis Y CAR T cells isolated from the tumors versus spleens of OVCAR3 tumor bearing mice following treatment and identifies genes selectively expressed in the tumor. (A) A graphical representation of fold change (y-axis; log 2(FC)) and mean expression (x-axis) in CD8⁺ and CD4⁺ CAR T cells isolated from the tumor site relative to CD8+ CAR T cells isolated from the spleen. (B) A graphical representation of normalized CPMs for PDCD1 and NR4A2 in CD8⁺ or CD4⁺ CAR T cells isolated from the tumor site relative to CD8⁺ and CD4⁺ CAR T cells isolated from the spleen and the pretreatment CAR T cell product that comprised a mixture of CD8+ and CD4+ CAR T cells. (C) The expression of PD-1 (x-axis) and CD69 (y-axis) in CD8+CAR T cells isolated from the spleens and tumors of OVCAR3 tumor bearing mice at day 9 post treatment. Data represents a concatenated sample from n=6 mice.

FIG. 20 shows that CRISPR-HDR editing of CAR T cells to integrate NGFR at the NR4A2 target loci results in CAR activation-dependent expression of NGFR in vitro and in vivo. (A) Representative FACS plots showing NGFR (y-axis) and CD69 (x-axis) expression by CRISPR-HDR edited CD8+ CAR T cells co-cultured with the OVCAR3, MCF7 and MDA-MB435 tumor cell lines. (B) A graphical representation of NGFR expression (y-axis; percent NGFR+) by CRISPR-HDR edited CD8+(left panel) or CD4+(right panel) CAR T cells co-cultured with the OVCAR3, MCF7 and MDA-MB435 tumor cell lines (x-axis). Data shown as mean±SD of triplicate cultures. (C) Representative FACS plots of NGFR (y-axis) and CD69 (x-axis) expression by CRISPR-HDR edited CD8+ CAR T cells isolated from the tumors or spleens of tumor bearing NSG mice. (D) A graphical representation of NGFR (y-axis; percent NGFR+) expression by CRISPR-HDR edited CD8+ CAR T cells isolated from the tumors, liver or spleens of tumor bearing NSG mice Data represents the mean+/−SEM of 6 individual mice.

### BRIEF DESCRIPTION OF THE SEQUENCES

Nucleic acid sequences are referred to by sequence identified (SEQ ID NO) with reference to the accompanying Sequence Listing, in which:

SEQ ID NO: 1 is the nucleotide sequence encoding GFP under the control of an NFAT promoter.

SEQ ID NO: 2 is the nucleotide sequence of the NGRF/NFAT/GFP vector.

SEQ ID NO: 3 is the nucleotide sequence of an AAV6 vector.

SEQ ID NO: 4 is the nucleotide sequence of the murine Pdcd1 sgRNA.

SEQ ID NO: 5 is the nucleotide sequence of the murine NR4A2 sgRNA.

SEQ ID NO: 6 is the nucleotide sequence of the human PD-1 sgRNA

SEQ ID NO: 7 is the nucleotide sequence of the human NR4A2 sgRNA.

SEQ ID NO: 8 is the nucleotide sequence of the murine Pdcd1/GFP HDR template.

SEQ ID NO: 9 is the nucleotide sequence of the murine NR4A2/GFP HDR template.

SEQ ID NO: 10 is the nucleotide sequence of the murine Pdcd1/CXCL9 HDR template.

SEQ ID NO: 11 is the nucleotide sequence of the murine NR4A2/CXCL9 HDR template.

SEQ ID NO: 12 is the nucleotide sequence of the murine Pdcd1/CXCL10 HDR template.

SEQ ID NO: 13 is the nucleotide sequence of the murine NR4A2/CXCL10 HDR template.

SEQ ID NO: 14 is the nucleotide sequence of the murine Pdcd1/CXCL9-P2A-CXCL10 HDR template.

SEQ ID NO: 15 is the nucleotide sequence of the murine NR4A2/CXCL9-P2A-CXCL10 HDR template.

SEQ ID NO: 16 is the nucleotide sequence of the murine Pdcd1/TNF HDR template.

SEQ ID NO: 17 is the nucleotide sequence of the murine NR4A2/TNF HDR template.

SEQ ID NO: 18 is the nucleotide sequence of the murine Pdcd1/IFNγ HDR template.

SEQ ID NO: 19 is the nucleotide sequence of the murine NR4A2/IFNγ HDR template.

SEQ ID NO: 20 is the nucleotide sequence of the murine Pdcd1/TNF-P2A-IFNγ HDR template.

SEQ ID NO: 21 is the nucleotide sequence of the murine NR4A2/TNF-P2A-IFNγ HDR template.

SEQ ID NO: 22 is the nucleotide sequence of the human PD-1/NGFR HDR template.

SEQ ID NO: 23 is the nucleotide sequence of the human NR4A2/NGFR HDR template.

SEQ ID NO: 24 is the nucleotide sequence of the human PD-1/CXCL9 HDR template.

SEQ ID NO: 25 is the nucleotide sequence of the human NR4A2/CXCL9 HDR template.

SEQ ID NO: 26 is the nucleotide sequence of the human PD-1/CXCL10 HDR template.

SEQ ID NO: 27 is the nucleotide sequence of the human NR4A2/CXCL10 HDR template.

SEQ ID NO: 28 is the nucleotide sequence of the human PD-1/CXCL9-P2A-CXCL10 HDR template.

SEQ ID NO: 29 is the nucleotide sequence of the human NR4A2/CXCL9-P2A-CXCL10 HDR template.

SEQ ID NO: 30 is the nucleotide sequence of the human PD-1/TNF HDR template.

SEQ ID NO: 31 is the nucleotide sequence of the human NR4A2/TNF HDR template.

SEQ ID NO: 32 is the nucleotide sequence of the human PD-1/IFNγ HDR template.

SEQ ID NO: 33 is the nucleotide sequence of the human NR4A2/IFNγ HDR template.

SEQ ID NO: 34 is the nucleotide sequence of the human PD-1/TNF-P2A-IFNγ HDR template.

SEQ ID NO: 35 is the nucleotide sequence of the human NR4A2/TNF-P2A-IFNγ HDR template.

SEQ ID NO: 36 is the nucleotide sequence of a cDNA encoding human NGFR.

SEQ ID NO: 37 is the nucleotide sequence of a cDNA encoding human CXCL9.

SEQ ID NO: 38 is the nucleotide sequence of a cDNA encoding human CXCL10.

SEQ ID NO: 39 is the nucleotide sequence of a cDNA encoding human CXCL9 and CXCL10, separated by a P2A linker.

SEQ ID NO: 40 is the nucleotide sequence of a cDNA encoding human TNF.

SEQ ID NO: 41 is the nucleotide sequence of a cDNA encoding human IFNγ.

SEQ ID NO: 42 is the nucleotide sequence of a cDNA encoding human TNF and IFNγ, separated by a P2A linker.

SEQ ID NO: 43 is the nucleotide sequence encoding a P2A linker.

SEQ ID NO: 44 is the nucleotide sequence encoding a T2A linker.

SEQ ID NO: 45 is the nucleotide sequence encoding a poly(A) tail.

SEQ ID NO: 46 is the nucleotide sequence of a PAM.

### DETAILED DESCRIPTION

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. All patents, patent applications, published applications and publications, databases, websites and other published materials referred to throughout the entire disclosure, unless noted otherwise, are incorporated by reference in their entirety. In the event that there is a plurality of definitions for terms, those in this section prevail. Where reference is made to a URL or other such identifier or address, it is understood that such identifiers can change and particular information on the internet can come and go, but equivalent information can be found by searching the internet. Reference to the identifier evidences the availability and public dissemination of such information.

The articles “a”, “an” and “the” include plural aspects unless the context clearly dictates otherwise. Thus, for example, reference to “an allele” includes a single allele, as well as two or more alleles; reference to “a cell” includes a single cell, as well as two or more cells, and so forth.

As used herein, “and/or” refers to and encompasses any and all possible combinations of one or more of the associated listed items, as well as the lack of combinations when interpreted in the alternative (or).

The term “about”, as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about”.

Each embodiment in this specification is to be applied mutatis mutandis to every other embodiment, unless expressly stated otherwise.

Nucleotide and amino acid sequences are referred to by a sequence identifier number (i.e., SEQ ID NO:). The SEQ ID NOs: correspond numerically to the sequence identifiers <400>1 (SEQ ID NO: 1), <400>2 (SEQ ID NO: 2), etc. A sequence listing is provided after the claims. A list describing the SEQ ID NOs in the sequence listing is provided above under the section “Brief Description of the Sequences”.

All sequence identifiers (e.g., GenBank ID, EMBL-Bank ID, DNA Data Bank of Japan (DDBJ) ID, etc.) provided herein were current at the filing date.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements.

The term “optionally” is used herein to mean that the subsequent described feature may or may not be present or that the subsequently described event or circumstance may or may not occur. Hence the specification will be understood to include and encompass embodiments in which the feature is present and embodiments in which the feature is not present, and embodiment in which the event or circumstance occurs as well as embodiments in which it does not.

The present disclosure is predicated, at least in part, on the inventors' surprising finding that engineering T cells by introducing a heterologous nucleotide sequence encoding at least one immunomodulatory factor in-frame within an endogenous gene that is selectively expressed by T cells in response to tumor cell stimulation (e.g., at the tumor site) enables the tumor-site specific expression of immunomodulatory factors. Accordingly, the engineered T cells of the present disclosure, also referred to as “armored” T cells, are particularly useful for immunotherapy. In particular, the engineered T cells described herein may be used for the treatment of solid tumors, without the toxicity that has previously been associated with the use of “armored” T cells for the treatment of solid tumors.

Thus, in an aspect disclosed herein, there is provided an engineered T cell comprising a heterologous nucleotide sequence encoding at least one immunomodulatory factor, wherein the heterologous nucleotide sequence is introduced in-frame of an endogenous gene of the T cell encoding a tumor-specific factor under the control of endogenous regulatory elements, wherein expression of the heterologous nucleotide sequence is controlled by the endogenous regulatory elements of the endogenous gene.

### Engineered T Cells

In an embodiment, the engineered T cell is derived from a thymocyte, naïve T lymphocyte, immature T lymphocyte, mature T lymphocyte, resting T lymphocyte, activated T lymphocyte, a tumor infiltrating lymphocytes (TILs), helper T cells (i.e., a CD4⁺ T cell), cytotoxic T cell (i.e., a CD8⁺ T cell), a CD4⁺ CD8⁺ T cell, a CD4⁻CD8⁻ T cell, or any other subset of T cells known in the art.

In an embodiment, the T cell is derived from a mammalian donor. In another embodiment, the T cell is derived from a human donor.

The term “isolated” as used herein refers to a cell, which is substantially or essentially free from components that normally accompany or interact with it as found in its naturally occurring environment, e.g., whole blood. Methods for the isolation of T cells from whole blood would be known to persons skilled in the art, illustrative examples of which include the Ficoll-Paque method.

The engineered T cells described herein comprise a heterologous nucleotide sequence encoding at least one immunomodulatory factor.

The term “heterologous” as used herein refers to any material introduced from outside of a cell. For example, a nucleotide sequence that is not normally present in a T cell in the copy number, position or configuration. As such, the “heterologous nucleotide sequence” may also be referred to as a “transgene”.

The term “immunomodulatory factor” as used herein refers to any molecule typically expressed by an immune cell (e.g., T cell, natural killer (NK) cell, B cell) that provides a signal to stimulate or inhibit an immune response. Suitable immunomodulatory factors would be known to persons skilled in the art, illustrative examples of which include cytokines (e.g., TNF, IFNγ, IFNα, IFNβ, IL-12, IL-18, CXCL9, CXCL10, XCL1, CD40L), and co-stimulatory molecules (e.g., MHC class I molecule, OX40, CD27, CD28, CDS, ICAM-1, LFA-1 (CD1 1a/CD18), ICOS (CD278), 4-1BB (CD137)).

In an embodiment, the immunomodulatory factor is a cytokine selected from the group consisting TNF, IFNγ, IFNα, IFNβ, IL-12, IL-18, CXCL9, CXCL10, XCL1, CD40L, and combinations of the foregoing. In another embodiment, the cytokine is selected from the group consisting of TNF, CXCL9, CXCL10, IFNγ, and combinations of the foregoing.

As described elsewhere herein, the heterologous nucleotide sequence is introduced in-frame within an endogenous gene of the T cell encoding a tumor-specific factor under the control of endogenous regulatory elements, wherein expression of the heterologous nucleotide sequence is controlled by the endogenous regulatory elements of the endogenous gene. By positioning the heterologous nucleotide sequence in-frame within an endogenous gene encoding a tumor-specific factor, the expression of the heterologous nucleotide sequence (i.e., the at least one immunomodulatory factor) is tumor responsive and/or tumor site-specific. Accordingly, unlike previous approaches that have sought to “armor” immune cells to improve immunotherapies (e.g., for the treatment of solid tumors), the expression of the heterologous nucleotide sequence is not constitutive and therefore enables the systemic delivery of the engineered T cells (e.g., the adoptive transfer of the engineered T cells) without inducing significant toxicity. The selective expression of the immunomodulatory factor in a tumor responsive and/or tumor site-specific manner thus enables the expression of immunomodulatory factors that, e.g., enhance cytotoxicity, modulate the tumor microenvironment, engage host immunity, improve lymphocyte trafficking and engage DCs to improve the therapeutic efficacy of immunotherapies, such as CAR T cell therapies.

The term “endogenous” as used herein refers to any material from or produced inside a cell. Accordingly, an “endogenous gene” is a gene that is present in the donor T cell (i.e., a wild-type T cell).

The term “tumor-specific factor” refers to a gene that is selectively expressed by T cells in response to tumor cell stimulation (e.g., at the tumor site).

The term “regulatory elements” as used herein refers to the nucleotide sequences required for the expression of a gene. In certain embodiments, the regulatory elements comprises a promoter. In other embodiments, the regulatory elements comprise a promoter, an enhancer and/or other regulatory elements that are required for the expression of a gene.

In an embodiment, the endogenous gene is selected from the genes listed in Table 1.

In another embodiment, the endogenous gene is selected from PD-1 and NR4A2.

The term “in-frame” as used herein means that the heterologous nucleotide sequence is introduced within the open reading frame of the endogenous gene.

Methods for the integration of heterologous nucleotide sequences would be known to persons skilled in the art, illustrative examples of which include the use of biological agents for producing site-specific DNA breaks, e.g., CRISPR-associated protein endonucleases, endonucleases, ZFNs, meganucleases, etc.

The position of the heterologous nucleotide sequence within the endogenous gene may affect the expression of the endogenous gene. For example, integration of the heterologous nucleotide sequence adjacent to the translation start codon of the endogenous gene may disrupt the expression of the endogenous gene. By contrast, integration of the heterologous nucleotide sequence adjacent to the stop codon of the endogenous gene may preserve the expression of the endogenous gene.

In an embodiment, the heterologous nucleotide sequence is introduced at a position adjacent to the translation start codon of the endogenous gene.

The “translation start codon” is the first codon of an mRNA transcript translated by a ribosome. In an embodiment, the translation start codon of the target gene is ATG. The term “endogenous translation start codon” refers to a translation start codon that originated from the donor T cell (i.e., the wild-type T cell).

In an embodiment, the heterologous nucleotide sequence is introduced at a position no more than about 20 nucleotides upstream or downstream of the translation start codon of the endogenous gene. Accordingly, in an embodiment, the heterologous nucleotide sequence is introduced at the translation start codon of the endogenous gene or about 1, 2, 3, 4, 5, 6, 7, 8, 9, 10, 11, 12, 13, 14, 15, 16, 17, 18, 19, or 20 nucleotides upstream or downstream of the translation start codon of the endogenous gene.

In an embodiment, introduction of the heterologous nucleotide sequence disrupts expression of the endogenous gene.

The term “disrupts” as used herein means a reduction in the level of expression of the endogenous gene. In an embodiment, the level of expression of the endogenous gene is reduced by at least about 40% relative to a T cell without the heterologous nucleotide sequence (i.e., a wild-type T cell). Accordingly, the level of expression of the endogenous gene may be reduced by at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or effectively abolished to an undetectable level, i.e., 100%.

In another embodiment, the heterologous nucleotide sequence is introduced at a position adjacent to the stop codon of the endogenous gene.

In an embodiment, the heterologous nucleotide sequence is introduced at a position no more than about 20 base pairs upstream of the stop codon of the endogenous gene. Accordingly, in an embodiment, the heterologous nucleotide sequence is introduced at the stop codon of the endogenous gene or about 1, 2, 3, 4, 5, 6, 7, 8, 9, 10, 11, 12, 13, 14, 15, 16, 17, 18, 19, or 20 nucleotides upstream of the stop codon of the endogenous gene.

In an embodiment, the heterologous nucleotide sequence further comprises one or more or all of:
(a) a stop codon;
(b) a nucleotide sequence encoding a poly(A) tail; and
(c) a linker.

The term “stop codon” as used herein refers to a codon that signals the end of the nucleotide sequence during translation. Accordingly, termination occurs when a ribosome reaches a stop codon (e.g., UAG, UAA and UGA).

The term “linker” refers to any nucleotide or group of nucleotides that binds two components of the heterologous nucleotide sequence. Suitable linkers would be known to persons skilled in the art, illustrative examples of which include a P2A linker (e.g., SEQ ID NO: 43) and a T2A linker (e.g., SEQ ID NO: 44).

Linkers may enable the integration of heterologous nucleotide sequences encoding two or more immunomodulatory factors, which are separated from each other to enable the transcription and translation of the two or more immunomodulatory factors. Alternatively, linkers may enable the positioning of the heterologous nucleotide sequence into a position adjacent to the, e.g., stop codon of the endogenous gene, without disrupting the expression of the endogenous gene. The linker can be of any length, but is typically at least 20 nucleotides in length. Accordingly, in an embodiment, the linker is at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100 nucleotides in length, and so on and so forth.

In an embodiment, the linker is selected from a P2A linker and a T2A linker.

A “poly(A) tail” is a polyadenylation signal comprising about 200 adenylate nucleotides at the 3′ end of a heterologous or endogenous mRNA. Suitable nucleotide sequences encoding a poly(A) tail would be known to persons skilled in the art, illustrative examples of which include the nucleotide sequence of SEQ ID NO: 45.

In an embodiment, the heterologous nucleotide sequence comprises a sequence of any one of SEQ ID NOs: 36 to 42, or a sequence that is at least 80% identical to the sequence of any one of SEQ ID NOs: 36 to 42. Accordingly, the sequence may be at least 80%, at least 81%, at least 82%, at least 83%, at least 84%, at least 85%, at least 86%, at least 87%, at least 89%, at least 90%, at least 91%, at least 92%, at least 93%, at least 94%, at least 95%, at least 96%, at least 97%, at least 98%, at least 99%, or 100% identical to the nucleic acid sequence of SEQ ID NOs: 36 to 42. Methods for the determination of nucleic acid sequence identity would be known to persons skilled in the art, illustrative examples of which include computer programs that employ algorithms such as BLAST (Altschul et al., 1990, Journal of Molecular Biology, 215(3): 403-410).

In an embodiment, the engineered T cell further comprises a chimeric antigen receptor (CAR).

The terms “chimeric antigen receptor” or “CAR” as used herein mean a recombinant polypeptide comprising at least an antigen-binding domain that is linked, via hinge and transmembrane domains, to an intracellular signaling domain.

The antigen-binding domain is a functional portion of the CAR that is responsible for transmitting information within the cell to regulate cellular activity via defined signaling pathways. In an embodiment, the antigen-binding domain may comprise an antibody or antibody fragment thereof.

The term “antibody” is used herein in the broadest sense and specifically covers monoclonal antibodies (including full length monoclonal antibodies), polyclonal antibodies, chimeric antibodies, humanized antibodies, human antibodies, multi-specific antibodies (e.g., bispecific antibodies), and single variable domain antibodies so long as they exhibit the desired biological activity. The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (which may be abbreviated as HCVR or V_{H}) and a heavy chain constant region. The heavy chain constant region comprises three domains, C_{H1}, C_{H2} and C_{H3}. Each light chain comprises a light chain variable region (which may be abbreviated as LCVR or VL) and a light chain constant region. The light chain constant region comprises one domain (C_{L1}). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments disclosed herein, the FRs of an antibody (or antigen-binding portion thereof) may be identical to the human germline sequences, or may be naturally or artificially modified. An amino acid consensus sequence may be defined based on a side-by-side analysis of two or more CDRs. Included within the scope of the term “antibody” is an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, TgE, IgG, and TgM, and several of these may be further divided into subclasses (isotypes), e.g., IgG1, IgG2, IgG3, IgG4, IgA1 and IgA2. The heavy-chain constant regions that correspond to the different classes of immunoglobulins are called α, δ, Σ, γ, and μ, respectively. The subunit structures and three-dimensional configurations of different classes of immunoglobulins are well known.

An “antigen-binding fragment” may be provided by means of arrangement of one or more CDRs on non-antibody protein scaffolds. “Protein scaffold” as used herein includes but is not limited to an immunoglobulin (Ig) scaffold, for example an IgG scaffold, which may be a four chain or two chain antibody, or which may comprise only the Fc region of an antibody, or which may comprise one or more constant regions from an antibody, which constant regions may be of human or primate origin, or which may be an artificial chimera of human and primate constant regions. The protein scaffold may be an Ig scaffold, for example an IgG, or IgA scaffold. The IgG scaffold may comprise some or all the domains of an antibody (i.e., CH1, CH2, CH3, V_{H}, V_{L}). The antigen binding protein may comprise an IgG scaffold selected from IgG1, IgG2, IgG3, IgG4 or IgG4PE. For example, the scaffold may be IgG1. The scaffold may consist of, or comprise, the Fc region of an antibody, or is a part thereof. Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment,” as used herein. An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain. In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) V_{H}-C_{H1}; (ii) V_{H}-C_{H2}; (iii) V_{H}-C_{H3}; (iv) V_{H}-C_{H1}-C_{H}2; (v) V_{H}-C_{H1}-C_{H2}-C_{H3}, (vi) V_{H}-C_{H2}-C_{H3}; (vii) V_{H}-C_{L}; (viii) V_{L}-C_{H}1; (ix) V_{L}-C_{H2}, (x) V_{L}-C_{H3}; (xi) V_{L}-C_{H1}-C_{H2}; (xii) V_{L}-C_{H1}-C_{H2}-C_{H3}; (xiii) V_{L}-C_{H2}-C_{H3}; and (Xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)). As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antigen-binding molecule format, including the exemplary bispecific antigen-binding molecule formats disclosed herein, may be adapted for use in the context of an antigen-binding fragment of an antibody of the present invention using routine techniques available in the art.

In an embodiment, the antigen-binding domain comprises an antibody fragment. For example, the antigen-binding domain may comprise a scFv consisting of a VL and VH sequence of a monoclonal antibody (mAb) specific for a tumor cell surface molecule (i.e., tumor antigen).

In an embodiment, the CAR binds an antigen selected from the group consisting of CD19, CD20, CD22, CD30, ROR1, CD123, CD33, CD133, CD138, GD2, Her2, Her1, mesothelin, MUC1, gp100, MART-1, MAGE-A3, MUC16, NY-ESO-1, L1-CAM, CEA, FAP, VEGFR2, WT1, TAG-72, CD171, α-FR, CAIX, PSMA, EGFRvIII CLL-1, GRP78, claudin 6, claudin 18.2 and Lewis Y. In another embodiment, the CAR binds an antigen selected from Her2 and Lewis Y.

In an embodiment, the CAR comprises at least one signaling domain. In another embodiment, the CAR comprises at least two signaling domains.

Examples of CAR signaling domains include CD3ζ, CD28, 41BB, DAP10, OX40, ICOS, DAP12, KIR2DS2, 4-1BB, CD3s, CD35, CD3C, CD25, CD27, CD79A, CD79B, CARD11, FcRa, Fcftp, FcRy, Fyn, HVEM, Lck, LAG3, LAT, LRP, NKG2D, NOTCH1, NOTCH2, NOTCH3, NOTCH4, ROR2, Ryk, SLAMFI, Slip76, pTa, TCRa, TCRP, TRIM, Zap70, PTCH2 and LIGHT.

In an embodiment, the CAR comprises a signaling domain selected from the group consisting of the CD28 and CD3ζ signaling domains. In another embodiment, the CAR comprises both the CD28 and CD3ζ signaling domains.

### Pharmaceutical Compositions

In an aspect of the present disclosure, there is provided a pharmaceutical composition comprising the engineered T cell (i.e., a population of the engineered T cells) described herein.

In an embodiment, the pharmaceutical composition comprises a population of the engineered T cells in sufficient number to administer a dosage of 10⁴ to 10⁹ cells/kg body weight. In another embodiment, the pharmaceutical composition comprises a population of the engineered T cells in sufficient number to administer a dosage of 10⁵ to 10⁶ cells/kg body weight, including all integer values within those ranges.

In some embodiments, periodic re-administration of the pharmaceutical composition may be required to achieve a desirable therapeutic effect. The exact amounts and rates of administration of the pharmaceutical composition will depend on a number of factors, examples of which are described elsewhere herein, such as the subject's age, body weight, general health, sex and dietary requirements, as well as any drugs or agents used in combination or coincidental with the administration of the composition. Where multiple divided doses are required, these may be administered hourly, daily, weekly, monthly or at other suitable time intervals or the dose may be proportionally reduced as indicated by the exigencies of the situation. Alternatively, a continuous infusion strategy can be employed.

In an embodiment, the pharmaceutical composition is suitable for parenteral administration. In another embodiment, the composition is suitable for intravenous administration.

The pharmaceutical compositions disclosed herein may be prepared according to conventional methods well known in the pharmaceutical industries, such as those described in Remington's Pharmaceutical Handbook (Mack Publishing Co., NY, USA), comprising a therapeutically effective amount of the composition alone, with one or more pharmaceutically acceptable carriers or diluents.

The term “pharmaceutically acceptable carrier” as used herein means any suitable carriers, diluents or excipients. These include all aqueous and non-aqueous isotonic sterile injection solutions, which may contain anti-oxidants, buffers and solutes to render the composition isotonic with the blood of the intended recipient, aqueous and non-aqueous sterile suspensions, which may include suspending agents and thickening agents, dispersion media, anti-fungal and anti-bacterial agents, isotonic and absorption agents, and the like.

In an embodiment, the pharmaceutical composition further comprises one or more immune adjuvants.

The term “immune adjuvant” as used herein refers to a compound or substance that is capable of enhancing a subject's immune response to the immunogen including, for example, the subject's antibody response to the immunogen. An immune adjuvant may therefore assist to enhance the immune response to an engineered T cell in a subject, compared to the administration of the engineered T cell or in the absence of the immune adjuvant.

Suitable immune adjuvants will be familiar to persons skilled in the art, illustrative examples of which include an inhibitor of the PDL-1: PD-1 axis, a TLR3 agonist, a 4-1BB agonist, a TLR7 agonist, an inhibitor of TJM-3, and an inhibitor of CTLA-4.

It is further contemplated herein that the pharmaceutical composition may be co-administered with one or more other agents suitable for the treatment or amelioration of symptoms associated with cancer, such as a solid tumor, illustrative examples of which include surgery, chemotherapy (e.g., anastrozole, bicalutamide, bleomycin sulfate, busulfan, busulfan injection, capecitabine, N4-pentoxycarbonyl-5-deoxy-5-fluorocytidine, carboplatin, carmustine, chlorambucil, cisplatin, cladribine, cyclophosphamide, cytarabine, cytosine arabinoside, cytarabine liposome injection, dacarbazine, dactinomycin, daunorubicin hydrochloride, daunorubicin citrate liposome injection, dexamethasone, docetaxel, doxorubicin hydrochloride, etoposide, fludarabine phosphate, 5-fluorouracil, flutamide, tezacitibine, gemcitabine, hydroxyurea, idarubicin, ifosfamide, irinotecan, L-asparaginase, leucovorin calcium, melphalan, 6-mercaptopurine, methotrexate, mitoxantrone, mylotarg, paclitaxel, phoenix (Yttrium90/MX-DTPA), pentostatin, polifeprosan 20 with carmustine implant, tamoxifen citrate, teniposide, 6-thioguanine, thiotepa, tirapazamine, topotecan hydrochloride for injection, vinblastine, vincristine, and vinorelbine), radiation, immunosuppressive agents (e.g., cyclosporin, azathioprine, methotrexate, mycophenolate, and FK506), antibodies, or other immunoablative agents (e.g., CAMPATH), targeted agents, steroids, and peptide vaccines.

Such combinations may be administered simultaneous with the pharmaceutical composition or concurrently with the pharmaceutical composition.

### Methods of Treatment and Associated Therapeutic Uses

In an aspect, the present disclosure provides a method for the treatment of cancer comprising the administration of a therapeutically effective amount of the engineered T cell or the pharmaceutical composition disclosed herein to a subject in need thereof.

In another aspect, the present disclosure provides the use of the engineered T cell or the pharmaceutical composition described herein in the manufacture of a medicament for the treatment of cancer.

In an embodiment, the engineered T cell described herein is for use in the treatment of cancer.

The therapeutic regimen for the treatment of cancer can be determined by a person skilled in the art and will typically depend on factors including, but not limited to, the type, size, stage and receptor status of the tumor in addition to the age, weight and general health of the subject. Another determinative factor may be the risk of developing recurrent disease. For instance, for a subject identified as being at high risk or higher risk or developing recurrent disease, a more aggressive therapeutic regimen may be prescribed as compared to a subject who is deemed at a low or lower risk of developing recurrent disease. Similarly, for a subject identified as having a more advanced stage of cancer, for example, stage III or IV disease, a more aggressive therapeutic regimen may be prescribed as compared to a subject that has a less advanced stage of cancer.

The term “cancer” as used herein means any condition associated with aberrant cell proliferation. Such conditions will be known to persons skilled in the art. In an embodiment, the cancer is a primary cancer (e.g., a tumor). In another embodiment, the cancer is a metastatic cancer.

Examples of various cancers are described elsewhere herein and include breast cancer, colorectal cancer, prostate cancer, ovarian cancer, cervical cancer, skin cancer, pancreatic cancer, renal cancer, liver cancer, brain cancer, lymphoma, leukemia, lung cancer, sarcoma and the like. The terms “cancer” and “tumor” may be used interchangeably herein, e.g., encompassing both solid and diffuse or circulating tumors.

In an embodiment, the cancer is a solid cancer.

In an embodiment, the solid cancer is selected from the group consisting of colorectal cancer, breast cancer, melanoma and sarcoma.

The term “subject” as used herein refers to any mammal, including livestock and other farm animals (such as cattle, goats, sheep, horses, pigs and chickens), performance animals (such as racehorses), companion animals (such as cats and dogs), laboratory test animals and humans. In an embodiment, the subject is a human. In an embodiment, the subject is an adult. In another embodiment, the subject is a child.

As used herein, the term “effective amount” typically refers to an amount of the engineered T cell or pharmaceutical composition described herein that is sufficient to affect one or more beneficial or desired therapeutic outcomes (e.g., reduction in tumor size). Said beneficial or desired therapeutic outcomes may be measured using clinical techniques known in the art, illustrative examples of which include the measurement of imaging biomarkers, tumor size (e.g., as measured by anatomical imaging modalities, such as CT or MRI), quantification of the presence of inflammatory mediators (e.g., Interleukin-1, TNF, TGF-β, etc.). An “effective amount” can be provided in one or more administrations. The exact amount required may vary depending on factors such as the nature and severity of the cancer to be treated, and the age and general health of the subject.

The terms “treat”, “treating”, “treatment” and the like are used interchangeably herein to mean relieving, reducing, alleviating, ameliorating or otherwise inhibiting the severity and/or progression of cancer, or a symptom thereof, in a subject. It is to be understood that the terms “treat”, “treating”, “treatment” and the like, as used herein, do not imply that a subject is treated until clinical symptoms of cancer have been eliminated or are no longer evident (e.g., elimination of solid tumor mass and associated metastatic lesions, if any). Said treatment may also reduce the severity of cancer by preventing progression or alleviating the symptoms associated with cancer.

The terms “prevent”, “preventing”, “prevention” and the like are used interchangeably herein to mean inhibit, hinder, retard, reduce or otherwise delay the development of cancer and/or progression of cancer, or a symptom thereof, in a subject. In the context of the present disclosure, the term “prevent” and variations thereof does not necessarily imply the complete prevention of the specified event. Rather, the prevention may be to an extent, and/or for a time, sufficient to produce the desired effect. Prevention may be inhibition, retardation, reduction or otherwise hindrance of the event, activity or function. Such preventative effects may be in magnitude and/or be temporal in nature.

In an embodiment, the engineered T cell is an autologous engineered T cell.

The term “autologous” as used herein refers to any material derived from the same subject to whom it is later to be administered into the subject in accordance with the methods disclosed herein. Accordingly, in certain embodiments, T cells isolated from the subject may be contacted with the genome editing systems described herein and cultured ex vivo for a time and under conditions suitable for the integration of the heterologous nucleotide sequence, before being reinfused back into the subject in accordance with the method of treatment described herein.

In another embodiment, the engineered T cell is an allogenic engineered T cell.

The term “allogenic” as used herein refers to any material derived from a different animal of the same species as the subject to whom the material is administered.

### Genome Editing Systems

The present inventors' have generated the engineered T cells of the present disclosure by the precise integration of the heterologous nucleotide sequence using CRISPR-Cas9 gene editing. In particular, the use of a CRISPR-Cas9 genome editing system has enabled the integration of a heterologous nucleotide sequence encoding an immunomodulatory factor into the open reading frame of an endogenous gene encoding a tumor-specific facto under the control of endogenous regulatory elements. As such, the expression of the immunomodulatory factor by the engineered T cells is also controlled by the endogenous regulatory elements of the endogenous gene, thereby enabling the tumor-site specific expression of the immunomodulatory factor, thus avoiding the toxicity associated with the constitutive expression of immunomodulatory factors that is observed with typical approaches for the production of armored CAR cells.

Accordingly, in an aspect disclosed herein, there is provided a genome editing system comprising:
(a) a sgRNA comprising a sequence of at least 10 contiguous nucleotides that are complementary to a target nucleic acid sequence within an endogenous gene of a T cell encoding a tumor-specific factor under the control of endogenous regulatory elements;
(b) a RNA-guided nuclease; and
(c) a homology directed repair (HDR) template, wherein the HDR template comprises a nucleotide sequence encoding at least one inflammatory mediator.

The term “genome editing” as used herein refers to a type of genetic alteration in which a heterologous nucleotide sequence is inserted into a target nucleic acid sequence within an endogenous gene of a T cell encoding a tumor-specific factor, i.e., into the genome of a T cell, using one or more RNA-guided nucleases. Any suitable RNA-guided nuclease can be introduced into a T cell to induce genome editing of a target nucleic acid sequence, including CRISPR-associated protein (Cas) endonucleases, zinc finger nucleases (ZFNs), transcription activator-like effector nucleases (TALENs), meganucleases, variants, fragments and combinations thereof. Naturally-occurring and synthetic RNA-guided endonucleases are contemplated herein.

In an embodiment, the genome editing system is a CRISPR-Cas genome editing system.

The “clustered regularly interspaced short palindromic repeat” (CRISPR)/“CRISPR-associated protein” (Cas) system (CRISPR/Cas system) evolved in bacteria and archaea as an adaptive immune system to defend against viral attack. The mechanisms of CRISPR-mediated gene editing would be known to persons skilled in the art and have been described, for example, by Doudna et al., (2014, Methods in Enzymology, 546). Briefly, upon exposure to a virus, short segments of viral DNA are integrated in the clustered regularly interspaced short palindromic repeats (i.e., CRISPR) locus. RNA is transcribed from a portion of the CRISPR locus that includes the viral sequence. That RNA, which contains sequence complementarity to the viral genome, mediates targeting of a Cas endonuclease to the sequence in the viral genome. The Cas endonuclease cleaves the viral target sequence to prevent integration or expression of the viral sequence.

CRISPR-Cas genome editing systems may be used to generate a site-specific double strand break (DSB) or single strand break (SSB) within a double-stranded DNA (dsDNA). Once a DSB or SSB is detected in a cell, the DNA repair machinery will repair the break by “non-homologous end-joining” or “NHEJ” or “homology-directed repair” or “HDR”.

NHEJ is triggered to repair double-stranded breaks in which the break ends are directly ligated without the need for a homologous template. Due to the error-prone nature of this repair pathway, small insertions or deletions (INDELs) may be introduced at the target locus near the site of the initial cleavage, and such INDELs can cause frameshift mutations, promote internal ribosomal entry, convert pseudo-mRNAs into protein encoding molecules, or induce exon skipping by disruption of exon splicing enhancers (see, e.g., Tuladhar et al., 2019, Nature Communications, 10: 4056). Unpredicted large genome modifications can also be introduced, which can be more than several kilobases (see, e.g., Kosicki et al., 2018 Nature Biotechnology, 36: 765-771).

By contrast, HDR accurately and precisely repairs DNA breaks using a homologous template to guide repair. The most common form of HDR is homologous recombination (HR), by which nucleotide sequences are exchanged between two similar or identical molecules of DNA.

As used herein the terms “polynucleotide”, “nucleotide sequence”, “nucleic acid” or “nucleic acid molecule” mean a single- or double-stranded polymer of deoxyribonucleotide, ribonucleotide bases or known analogues or natural nucleotides, or mixtures thereof, and can include molecules comprising coding and non-coding sequences of a gene, sense and antisense sequences and complements, exons, introns, genomic DNA, cDNA, pre-mRNA, mRNA, rRNA, siRNA, miRNA, tRNA, ribozymes, recombinant polypeptides, isolated and purified naturally occurring DNA or RNA sequences, synthetic RNA and DNA sequences, nucleic acid probes, primers and fragments.

The term “target nucleic acid sequence” as used herein refers to a nucleic acid sequence within an endogenous gene of a T cell encoding a tumor-specific factor to which a gRNA is designed to have complementarity, where hybridization between the target nucleic acid sequence and the gRNA promotes the formation of a complex comprising the RNA-guided nuclease, the gRNA and the target nucleic acid sequence (i.e., a genome editing complex).

By “complementary” or “substantially complementary” it is meant that a nucleic acid (e.g., RNA, DNA) comprises a sequence of nucleotides that enables it to non-covalently bind, i.e., form Watson-Crick base pairs and/or G/U base pairs, “anneal”, or “hybridize” to another nucleic acid in a sequence-specific, antiparallel, manner (i.e., a nucleic acid specifically binds to a complementary nucleic acid) under the appropriate in vitro and/or in vivo conditions of temperature and solution ionic strength. Standard Watson-Crick base-pairing includes: adenine/adenosine (A) pairing with thymidine/thymidine (T), A pairing with uracil/uridine (U), and guanine/guanosine (G) pairing with cytosine/cytidine (C). In addition, for hybridization between two RNA molecules (e.g., dsRNA), and for hybridization of a DNA molecule with an RNA molecule (e.g., when a target nucleic acid sequence base pairs with a gRNA) G can also base pair with U. For example, G/U base-pairing is partially responsible for the degeneracy (i.e., redundancy) of the genetic code in the context of tRNA anti-codon base pairing with codons in rnRNA. Thus, in the context of this disclosure, a G (e.g., of a target nucleic acid sequence base pairing with a gRNA) is considered complementary to both a U and to C. For example, when a G/U base-pair can be made at a given nucleotide position of a protein binding segment of a guide RNA molecule, the position is not considered to be non-complementary, but is instead considered to be complementary.

Hybridization requires that the two nucleic acids contain complementary sequences, although mismatches between bases are possible. The conditions appropriate for hybridization between two nucleic acids depend on the length of the nucleic acids and the degree of complementarity, variables well known in the art. The greater the degree of complementarity between two nucleotide sequences, the greater the value of the melting temperature (Tₘ) for hybrids of nucleic acids having those sequences. Typically, the length for a hybridizable nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 30 nucleotides or more).

By “gene” it is meant a unit of inheritance that, when present in its endogenous state, occupies a specific locus on a genome and comprises transcriptional and/or translational regulatory sequences and/or a coding region and/or non-translated sequences (i.e., introns, 5′ and 3′ untranslated sequences).

As used herein, the terms “encode”, “encoding” and the like refer to the capacity of a nucleic acid to provide for another nucleic acid or a polypeptide. For example, a nucleic acid sequence is said to “encode” a polypeptide if it can be transcribed and/or translated to produce the polypeptide or if it can be processed into a form that can be transcribed and/or translated to produce the polypeptide. Such a nucleic acid sequence may include a coding sequence or both a coding sequence and a non-coding sequence. Thus, the terms “encode,” “encoding” and the like include an RNA product resulting from transcription of a DNA molecule, a protein resulting from translation of an RNA molecule, a protein resulting from transcription of a DNA molecule to form an RNA product and the subsequent translation of the RNA product, or a protein resulting from transcription of a DNA molecule to provide an RNA product, processing of the RNA product to provide a processed RNA product (e.g., mRNA) and the subsequent translation of the processed RNA product.

The terms “protein”, “peptide” and “polypeptide” are used interchangeably herein to refer to a polymer of amino acid residues linked together by peptide (amide) bonds. The terms refer to a protein, peptide, or polypeptide of any size, structure or function.

### Guide RNA

The terms “guide RNA” or “gRNA” refer to a RNA sequence that is complementary to a target nucleic acid sequence and directs a RNA-guided nuclease to the target nucleic acid sequence. gRNA typically comprises CRISPR RNA (crRNA) and a tracr RNA (tracrRNA). “crRNA” is a 17-20 nucleotide sequence that is complementary to the target nucleic acid sequence, while the “tracrRNA” provides a binding scaffold for the RNA-guided nuclease. crRNA and tracrRNA exist in nature a two separate RNA molecules, which has been adapted for molecular biology techniques using, for example, 2-piece gRNAs such as CRISPR tracer RNAs (cr:tracrRNAs).

The genome editing systems described herein comprise a sgRNA.

The terms “single-guide RNA” or “sgRNA” refer to a single RNA sequence that comprises the crRNA fused to the tracrRNA.

The sgRNA contemplated herein are complementary to a target nucleic acid sequence within an endogenous gene of a T cell encoding a tumor-specific factor under the control of endogenous regulatory elements.

In an embodiment, the sgRNA comprises a sequence of at least 10 contiguous nucleotides that are complementary to a target nucleic acid sequence. Accordingly, the sgRNA comprises a sequence of at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 nucleotides that are complementary to a target nucleic acid sequence.

In an embodiment, the sgRNA comprises a sequence of at least 20 contiguous nucleotides that are complementary to a target nucleic acid sequence.

Methods and tools for the design of sgRNA would be known to persons skilled in the art, illustrative examples of which include CHOPCHOP, CRISPR Design, sgRNA Designer, Synthego and GT-Scan.

In an embodiment, the endogenous gene is selected from the genes listed in Table 1. Suitable sgRNAs complementary to a target nucleic acid in any one or more of the genes listed in Table 1 could be designed and produced by persons skilled in the art, illustrative examples of which include the sgRNAs described herein, such as the sgRNA targeting PD-1 (SEQ ID NO: 6) or NR4A2 (SEQ ID NO: 7), as shown in Table 2.

### RNA-Guided Nuclease

In an embodiment, the RNA-guided nuclease is a CRISPR-associated (Cas) endonuclease. Suitable Cas endonucleases would be known to persons skilled in the art, illustrative examples of which include Cas3, Cas9, Cas12 (e.g., Casl2a, Casl2b, Casl2c, Cas12d, Casl2e) and Cas14.

In an embodiment, the RNA-guided nuclease is Cas9.

### Homology Directed Repair (HDR) Template

The HDR templates of the genome editing system disclosed herein comprise a nucleotide sequence encoding at least one immunomodulatory factor.

In an embodiment, the HDR template comprises a cDNA sequence encoding at least one immunomodulatory factor.

In an embodiment, the immunomodulatory factor is a cytokine selected from the group consisting of TNF, IFNγ, IFNα, IFNβ, IL-12, IL-18, CXCL9, CXCL10, XCL1, CD40L, and combinations of the foregoing.

In another embodiment, the cytokine is selected from the group consisting of TNF, CXCL9, CXCL10, IFNγ, and combinations of the foregoing.

Where combinations of two or more immunomodulatory factors are encoded, the nucleotide sequence encoding each immunomodulatory factor may be separated by a linker.

As described elsewhere herein, a linker is any nucleotide or group of nucleotides that binds two components of the HDR template. In an embodiment, the linker is selected from a P2A linker (e.g., SEQ ID NO: 43) and a T2A linker (e.g., SEQ ID NO: 44). In a preferred embodiment, the linker is a P2A linker (e.g., SEQ ID NO: 43).

The structure and arrangement of the HDR template will be determined by reference to the desired position of the alteration to be made to the target nucleic acid sequence. Typically, a HDR template comprises a pair of homology arms corresponding to the target nucleic acid sequence, wherein each pair of homology arms flanks one or more transgenes (i.e., nucleotide sequence encoding at least one immunomodulatory factor). In a particular example, the pair of homology arms comprises a 5′ homology arm and a 3′ homology arm. A “5′ homology arm” refers to a polynucleotide sequence that is identical, or nearly identical, or homologous to a DNA sequence 5′ of a target site (e.g., translation start codon or translation stop codon of an endogenous gene). A “3′ homology arm” refers to a polynucleotide sequence that is identical, or nearly identical, or homologous to a DNA sequence 3′ of the target site.

As described elsewhere herein, introduction of the heterologous nucleotide sequence adjacent to the translation start codon of an endogenous gene may disrupt the expression of the endogenous gene. This may be desirable where the endogenous gene encodes an immunosuppressive factor.

Accordingly, in an embodiment, the target nucleic acid sequence comprises or is adjacent to the translation start codon of the endogenous gene.

In an embodiment, the translation start codon of the endogenous gene is within from about 10 nucleotides to about 50 nucleotides upstream or downstream from a protospacer adjacent motif (PAM).

The terms “protospacer adjacent motif” and “PAM” as used herein refer to a nucleotide sequence adjacent to the target nucleic acid sequence. In an embodiment, the PAM is the sequence 5′-NGG-3′ (SEQ ID NO: 46), where N is any nucleotide (i.e., A, T, C, G).

In an embodiment, the HDR template further comprises one or more or all of:
(a) a 5′ homology arm comprising a sequence of from about 250 to about 600 contiguous nucleotides that are homologous to a region 5′ to the translation start codon of the endogenous gene;
(b) a stop codon;
(c) a nucleotide sequence encoding a poly(A) tail;
(d) a linker; and
(e) a 3′ homology arm comprising a sequence of from about 250 to about 600 contiguous nucleotides that are homologous to a region 3′ to the translation start codon of the endogenous gene.

In an embodiment, the 5′ homology arm comprises a sequence of from about 250 to about 600 contiguous nucleotides that are homologous to a region immediately 5′ to the translation start codon of the endogenous gene, preferably about 250, preferably about 260, preferably about 270, preferably about 280, preferably about 290, preferably about 300, preferably about 310, preferably about 320, preferably about 330, preferably about 340, preferably about 350, preferably about 360, preferably about 370, preferably about 380, preferably about 390, preferably about 400, preferably about 410, preferably about 420, preferably about 430, preferably about 440, preferably about 450, preferably about 460, preferably about 470, preferably about 480, preferably about 490, preferably about 500, preferably about 510, preferably about 520, preferably about 530, preferably about 540, preferably about 550, preferably about 560, preferably about 570, preferably about 580, preferably about 590, or more preferably about 600 contiguous nucleotides that are homologous to a region immediately 5′ to the translation start codon of the endogenous gene.

In another embodiment, the 5′ homology arm comprising a sequence of from about 350 to about 500 contiguous nucleotides that are homologous to a region 5′ to the translation start codon of the endogenous gene.

In an embodiment, the 3′ homology arm comprises a sequence of from about 250 to about 600 contiguous nucleotides that are homologous to a region immediately 3′ to the translation start codon of the endogenous gene, preferably about 250, preferably about 260, preferably about 270, preferably about 280, preferably about 290, preferably about 300, preferably about 310, preferably about 320, preferably about 330, preferably about 340, preferably about 350, preferably about 360, preferably about 370, preferably about 380, preferably about 390, preferably about 400, preferably about 410, preferably about 420, preferably about 430, preferably about 440, preferably about 450, preferably about 460, preferably about 470, preferably about 480, preferably about 490, preferably about 500, preferably about 510, preferably about 520, preferably about 530, preferably about 540, preferably about 550, preferably about 560, preferably about 570, preferably about 580, preferably about 590, or more preferably about 600 contiguous nucleotides that are homologous to a region immediately 3′ to the translation start codon of the endogenous gene.

In another embodiment, the 3′ homology arm comprises a sequence of from about 350 to about 500 contiguous nucleotides that are homologous to a region 3′ to the translation start codon of the endogenous gene.

In an embodiment, the HDR template comprises a sequence of any one of SEQ ID NOs: 22 to 35, or a sequence that is at least 80% identical to the sequence of any one of SEQ ID NOs: 22 to 35. Accordingly, the sequence may be at least at least 80%, at least 81%, at least 82%, at least 83%, at least 84%, at least 85%, at least 86%, at least 87%, at least 89%, at least 90%, at least 91%, at least 92%, at least 93%, at least 94%, at least 95%, at least 96%, at least 97%, at least 98%, at least 99%, or 100% identical to the sequence of any one of SEQ ID NOs: 22 to 35.

As described elsewhere herein, introduction of the heterologous nucleotide sequence adjacent to the stop codon of the endogenous gene may preserve the expression of the endogenous gene, which may be beneficial where the endogenous gene is essential or required for T cell function.

Accordingly, in an embodiment, the target nucleic acid sequence comprises or is adjacent to the stop codon of the endogenous gene.

In an embodiment, the HDR template further comprises one or more of all of:
(a) a 5′ homology arm comprising a sequence of from about 250 to about 600 contiguous nucleotides that are homologous to a region 5′ to the stop codon of the endogenous gene;
(b) a linker; and
(c) a 3′ homology arm comprising a sequence of from about 250 to about 600 contiguous nucleotides that are homologous to a region 3′ to the stop codon of the endogenous gene.

In an embodiment, the 5′ homology arm comprises a sequence of from about 250 to about 600 contiguous nucleotides that are homologous to a region immediately 5′ to the stop codon of the endogenous gene, preferably about 250, preferably about 260, preferably about 270, preferably about 280, preferably about 290, preferably about 300, preferably about 310, preferably about 320, preferably about 330, preferably about 340, preferably about 350, preferably about 360, preferably about 370, preferably about 380, preferably about 390, preferably about 400, preferably about 410, preferably about 420, preferably about 430, preferably about 440, preferably about 450, preferably about 460, preferably about 470, preferably about 480, preferably about 490, preferably about 500, preferably about 510, preferably about 520, preferably about 530, preferably about 540, preferably about 550, preferably about 560, preferably about 570, preferably about 580, preferably about 590, or more preferably about 600 contiguous nucleotides that are homologous to a region immediately 5′ to the stop codon of the endogenous gene.

In another embodiment, the 5′ homology arm comprising a sequence of from about 350 to about 500 contiguous nucleotides that are homologous to a region 5′ to the stop codon of the endogenous gene.

In an embodiment, the 3′ homology arm comprises a sequence of from about 250 to about 600 contiguous nucleotides that are homologous to a region immediately 3′ to the stop codon of the endogenous gene, preferably about 250, preferably about 260, preferably about 270, preferably about 280, preferably about 290, preferably about 300, preferably about 310, preferably about 320, preferably about 330, preferably about 340, preferably about 350, preferably about 360, preferably about 370, preferably about 380, preferably about 390, preferably about 400, preferably about 410, preferably about 420, preferably about 430, preferably about 440, preferably about 450, preferably about 460, preferably about 470, preferably about 480, preferably about 490, preferably about 500, preferably about 510, preferably about 520, preferably about 530, preferably about 540, preferably about 550, preferably about 560, preferably about 570, preferably about 580, preferably about 590, or more preferably about 600 contiguous nucleotides that are homologous to a region immediately 3′ to the stop codon of the endogenous gene.

In another embodiment, the 3′ homology arm comprising a sequence of from about 350 to about 500 contiguous nucleotides that are homologous to a region 3′ to the stop codon of the endogenous gene.

As described elsewhere herein, linkers can enable the positioning of the heterologous nucleotide sequence into a position adjacent to the, e.g., stop codon of the endogenous gene, without disrupting the expression of the endogenous gene. Accordingly, in an embodiment, the heterologous nucleotide sequence encoding at least one immunomodulatory factor is positioned downstream of linker, which is integrated upstream of the stop codon of the endogenous gene.

The HDR templates contemplated herein may further comprise one or more additional modifications to, e.g., to prevent sgRNA/Cas9 RNP binding, disrupt expression of the endogenous gene and/or to introduce or remove a PAM. For example, the C nucleotide at position 1173 of SEQ ID NO: 24 differs from the corresponding G nucleotide in the homologous sequence of the target gene to prevent sgRNA/Cas9 RNP binding; and the TGA nucleotide sequence at positions 1184-1186 of SEQ ID NO: 24 differs from the corresponding AAG nucleotides in the homologous sequence of the target gene to: (i) prevent sgRNA/Cas9 RNP binding, and (ii) disrupt expression of the endogenous gene (i.e., PD-1) following genome editing.

### Vectors, Delivery Vehicles and Cells

In an embodiment, the HDR template is within a vector.

The vectors can be episomal vectors (i.e., that do not integrate into the genome of a host cell), or can be vectors that integrate into a host cell genome. Vectors may be replication competent or replication-deficient. Exemplary vectors include, but are not limited to, plasmids, cosmids, and viral vectors, such as adeno-associated virus (AAV) vectors, lentiviral, retroviral, adenoviral, herpesviral, parvoviral and hepatitis viral vectors. The choice and design of an appropriate vector is within the ability and discretion of one of ordinary skill in the art. Preferably, however, the vector is suitable for use in biotechnology.

Vectors suitable for use in biotechnology would be known to persons skilled in the art, illustrative examples of which include viral vectors derived from adenovirus, adeno-associated virus (AAV), herpes simplex virus (HSV), retrovirus, lentivirus, self-amplifying single-strand RNA (ssRNA) viruses such as alphavirus (e.g., Semliki Forest virus, Sindbis virus, Venezuelan equine encephalitis, M1), and flavivirus (e.g., Kunjin virus, West Nile virus, Dengue virus), rhabdovirus (e.g., rabies, vesicular stomatitis virus), measles virus, Newcastle Disease virus (NDV) and poxivirus as described by, for example, Lundstrom (2019, Diseases, 6: 42).

In an embodiment, the vector is an adeno-associated virus (AAV) vector. Exemplary AAV vectors include, without limitation, those derived from serotypes AAV1, AAV2, AAV3, AAV4, AAV5, AAV6, AAV7, AAV8, AAV9, AAV10, AAV11, AAV12 or AAV13, or using synthetic or modified AAV capsid proteins such as those optimized for efficient in vivo transduction. A recombinant AAV vector describes replication-defective virus that includes an AAV capsid shell encapsidating an AAV genome. Typically, one or more of the wild-type AAV genes have been deleted from the genome in whole or part, preferably the rep and/or cap genes.

In an embodiment, the AAV vector is an AAV5 or AAV6 vector.

Where the HDR template is within a vector, the HDR template may further comprise one or more cut sites for cloning into a vector. For example, SEQ ID NO: 24 is flanked by NotI cut sites at the 5′ and 3′ end (i.e., nucleotides 1-8 and 1674-1651 of SEQ ID NO: 24).

The present disclosure also provides non-viral delivery vehicles of the genome editing systems as described herein, and components thereof. Suitable non-viral delivery vehicles will be known to persons skilled in the art, illustrative examples of which include using lipids, lipid-like materials or polymeric materials, as described by, e.g., Rui et al. (2019, Trends in Biotechnology, 37(3): 281-293), and nanoparticles/nanocarriers, as described by, e.g., Nguyen et al. (2020, Nature Biotechnology, 38: 44-49).

In an embodiment, the sgRNA and the RNA-guided nuclease are complexed as a ribonucleoprotein (RNP).

The terms “ribonucleoproteins”, “RNPs” and “Cas-gRNA ribonucleoproteins” are used interchangeably herein to refer to a complex comprising sgRNA and an RNA-guided nuclease that can be used to directly deliver the sgRNA and RNA-guided nuclease into a T cell.

Suitable methods for the direct delivery of RNPs to cells (e.g., T cells) would be known to persons skilled in the art, illustrative examples of which include microinjection and electroporation. In an embodiment, the RNP is delivered to cells by electroporation.

In an embodiment, the genome editing system further comprises an inhibitor of non-homologous end-joining (NHEJ).

Suitable inhibitors of NHEJ would be known to persons skilled in the art, illustrative examples of which include DNA ligase IV (e.g., SCR7) and DNA-PK (e.g., VX-984, NU7026, M3814) inhibitors.

In an embodiment, the inhibitor of NHEJ is a DNA-PK inhibitor. In another embodiment, the DNA-PK inhibitor is M3814.

In another aspect, the present disclosure provides a cell or a cell extract comprising the genome editing system as described herein.

Cells according to the present disclosure include any cell into which the polynucleotides, vectors and RNPs described herein may be introduced and expressed.

The cell or cell extract contemplated herein may be derived from any species, particularly a vertebrate, and even more particularly a mammal. Suitable vertebrates that fall within the scope of the disclosure include, but are not restricted to, any member of the subphylum Chordata including primates (e.g., humans, monkeys and apes, and includes species of monkeys such from the genus Macaca (e.g., cynomologus monkeys such as Macaca fascicularis, and/or rhesus monkeys (Macaca mulatta)) and baboon (Papio ursinus), as well as marmosets (species from the genus Callithrix), squirrel monkeys (species from the genus Saimiri) and tamarins (species from the genus Saguinus), as well as species of apes such as chimpanzees (Pan troglodytes)), rodents (e.g., mice rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars etc.), marine mammals (e.g., dolphins, whales), reptiles (snakes, frogs, lizards etc.), and fish. In a preferred embodiment, the cell or cell extract is derived from a human.

In particular embodiments, the cells are derived from human T cells.

### Methods for Altering a Target Nucleic Acid Sequence

In another aspect, the present disclosure provides a method of altering a nucleic acid molecule in a T cell, the method comprising providing to the T cell the genome editing system as described herein.

The term “altering” as used herein refers to any change to the target nucleic acid sequence, including the insertion of one or more nucleotides (e.g., a heterologous nucleotide sequence) into the target nucleic acid sequence.

The cell may be provided with the genome editing systems described herein using any suitable method known in the art. Such methods include transfection, transduction, viral transduction, microinjection, lipofection, nucelofection, nanoparticle bombardment, transformation, conjugation and the like. The skilled person would readily understand and adapt any such method taking consideration of whether the components of genome editing system are provided as polynucleotides, vectors or RNPs.

In an embodiment, the sgRNA and RNA-guided nuclease are provided to the T cell complexed as an RNP; and the HDR template is provided to the T cell within a vector.

Those skilled in the art will appreciate that the invention described herein is susceptible to variations and modifications other than those specifically described. It is to be understood that the invention includes all such variations and modifications, which fall within the spirit and scope. The invention also includes all of the steps, features, compositions and compounds referred to or indicated in this specification, individually or collectively, and any and all combinations of any two or more of said steps or features.

Unless otherwise defined, all technical and scientific terms used herein have the same meanings as commonly understood by one of ordinary skill in the art to which this invention belongs.

All patents, patent applications and publications mentioned herein are hereby incorporated by reference in their entireties.

The various embodiments enabled herein are further described by the following non-limiting examples.

### EXAMPLES

### Example 1—Materials and Methods

### NFAT-GFP Reporter System

CAR T cells engineered to express GFP under the control of an NFAT promoter sequence were generated by cloning the sequence of SEQ ID NO: 1 into a NGFR retroviral vector downstream of the stop codon of the NGFR gene in accordance with the method of Zhang et al. (2011, Molecular Therapy, 19(4): 751-759). The NGFR/NFAT/GFP vector is shown in SEQ ID NO: 2.

### RNA-Seq

Total RNA was isolated using an RNAeasy kit (Qiagen) as per manufacturer's instructions and RNA-seq libraries were prepared from ˜400 ng of total RNA using the Quant-seq 3′ mRNA-seq Library Prep Kit for Illumina (Lexogen) as per manufacturer's instructions. Single-end, 75 bp RNA-seq short reads were generated using NextSeq (Illumina, Inc., San Diego, CA). CASAVA 1.8.2 was used for base calling. Quality of the data was assessed using RNA-SeQC v1.1.7 (DeLuca et al., 2012, Bioinformatics, 28(11): 1530-2). To analyze differential gene expression, the data was quality trimmed using Cutadapt v1.6 to remove random primer bias and 3′ end trimming was performed to remove poly(A)-tail derived reads and alignment performed using HISAT2 against the mouse reference genome mm10 or the human genome hg38, as appropriate. The subread software package 1.6.4 was used to count the number of reads per gene using gene definitions from Ensembl Release 96 (Zerbino et al., 2018, Nucleic Acids Research, 46(D1): D754-D761). Gene counts were normalized using the TMM (trimmed means of M-values) method and converted into log 2 counts per million using EdgeR v 3.8.5 (Robinson et al., 2010, Bioinformatics, 26(1): 139-40; McCarthy et al., 2012, Nucleic Acids Research, 40(10): 4288-97). All differentially expressed genes were filtered for false discovery rate (FDR) cutoff of 5% and fold change cutoff >1.

### CRISPR/Cas9 Gene Editing

Per 1×10⁶ activated human T cells or 20×10⁶ activated murine T cells to be electroporated, 37 pmoles recombinant Cas9 (IDT) and 270 pmoles sgRNA (Synthego) were combined and incubated for 10 min at RT to generate Cas9/sgRNA RNP.

Human T cells (1×10⁶) or murine T cells (20×10⁶) were resuspended in 20 μL P3 Buffer (Lonza), combined with RNP and electroporated using a 4D-Nucleofector (Lonza) using pulse code CM137 (murine cells) or E0115 (human cells). Pre-warmed complete RPMI media was then immediately added to electroporation wells and T cells were then incubated with AAV6 (Packgene or Vigene) encoding CRISPR-HDR templates of interest for 4 hours at 37° C.

### Flow Cytometry

For immunofluorescence staining, morphology of cells was determined via FSC-A and SSC-A, doublets were excluded via FSC-A and FSC—H and dead cells excluded via Fixable-Yellow live/dead stain (Invitrogen, Carlsbad, CA, USA).

Extracellular staining: Cell suspensions in round-bottom 96-well plates were centrifuged at 1400 rpm for 4 minutes and supernatants were discarded. Cells were stained with a cocktail of fluorochrome-conjugated antibodies in a total volume of 30-50 μL/well for 30 minutes at 4° C. Cells were washed twice with 100 μL of flow cytometry buffer.

Intracellular staining: Surface stained cells that required intracellular staining were fixed and permeabilized using a Cytofix/Cytoperm Solution Kit according to the manufacturer's instructions (BD Pharmigen). Cells were washed twice in 200 μL of fixation/permeabilization buffer and centrifuged at 1400 rpm for 4 minutes and supernatants were discarded. Cells were stained with a cocktail of fluorochrome-conjugated antibodies in a total volume of 30-50 μL/well for 30 minutes at room temperature in the dark. Cells were washed again, and resuspended in 100 μL of fixation/permeabilization buffer for flow cytometry analysis.

Data was collected using a BD CANTO, BD Fortessa, or BD Symphony (BD Biosciences, San Jose, California, USA) and analysed using FacsDiva v8 and Flowlogic v10.2 software (Miltenyi Biotec, Auburn, California, USA).

### Live Cell Imaging

Immune cell killing assays were conducted using the Incucyte Live-Cell Analysis System to examine the dynamics of immune cell killing of cancer cell lines in vitro in accordance with the method described by Li et al. (2018, Cell Death & Disease, 9: 177).

### Chromium Release Assay

Target tumor cells were labelled with 100 μCi chromium-51 (⁵¹Cr) for 1 hour at 37° C. with 5% CO2. Tumor cells were washed three times with serum-free RPMI media and centrifuged at 1500 rpm for 4 minutes. Tumors were re-suspended in supplemented RPMI media and plated at 1×10⁴ cells/well in a V-bottom 96-well plate. T cells were added to tumor cells at indicated effector cell: target ratios (E:T ratios). Control wells containing ⁵¹Cr-labelled tumors cells alone or tumor cells with 100 μL of sodium dodecyl sulfate (SDS) were included to determine spontaneous and total chromium release, respectively. Plates were incubated for 4 hours at 37° C. with 5% CO₂. Following incubation, plates were centrifuged at 1500 rpm for 5 minutes and 100 μl of supernatant from each well was transferred to 1.2 mL microtiter tubes (Quality Scientific Plastics, San Diego, California, USA). Chromium release was measured using an automatic gamma counter Wallac Wizard 1470 (General Electricity Healthcare, Amersham Australia, KewDale, Western Australia).

Percentage of tumor cell lysis was calculated using the formula: [(sample counts per minute−spontaneous counts per minute)/(total counts per minute−spontaneous counts per minute)]×100%.

### OT-1 T Cell Model

Splenocytes were activated with anti-CD3 (0.5 μg/ml) and anti-CD28 (0.5 μg/mL) in the presence of 100 IU/mL IL-2 and 200 μg/mL IL-7 at a density of 5×10⁶ per milliliter. After 24 hours, live T cells were isolated after a Ficoll centrifugation step. T cells were then were CRISPR/Cas9 edited and maintained in IL-2 and IL-7 or IL-7 and IL-15 (10 ng/mL) and cells used at days 5-7 post-activation.

### Anti-Her2 CAR T Cell Model

Primary murine splenocytes were transduced with a CAR containing the CD28 and TCR-ζ signaling domains recognizing the human Her2 antigen (scFv-CD28ζ) as described in John et al. (2013, Clinical Cancer Research, 19: 5636-5646).

The GP+E86 anti-Her2 CAR packaging line was then further engineered to also produce retrovirus encoding a truncated human Nerve Growth Factor Receptor (NGFR) to allow tracking of transduced cells. Splenocytes were cultured in RPMI supplemented with 10% FCS, NEAA, sodium pyruvate, glutamine, HEPES and penicillin/streptomycin. Splenocytes were activated with anti-CD3 (0.5 μg/ml) and anti-CD28 (0.5 μg/ml) in the presence of 100 IU/mL IL-2 and 200 μg/mL IL-7 at a density of 5×10⁶ per milliliter. After 24 hours, live T cells were isolated after a Ficoll centrifugation step. Four milliliters of retroviral supernatant was added to each well of retronectin-coated (10 μg/ml) 6-well plates (Takara Bio). Viral supernatant was spun onto retronectin-coated plates at 1200 g for 30 minutes after which T cells were resuspended in 1 mL of additional retroviral-containing supernatant supplemented with IL-2 and IL-7 and then added to the retronectin-coated plates to give a final volume of 5 mL/well. Final T cell concentration was 5×10⁶ to 10×10⁶ per well. T cells were spun for 90 minutes, after which they were incubated overnight before repetition of the transduction process the following day. After the transduction was completed, T cells were maintained in IL-2 and IL-7 or IL-7 and IL-15 containing media and cells used at days 6-8 after transduction.

### Anti-Lewis Y CAR T Cell Model

Human PBMCs were activated with either anti-CD3 (OKT3, 30 ng/mL) and cultured in RPMI media supplemented with 10% FCS, penicillin/streptomycin, sodium pyruvate, glutamax, NEAA and HEPES and IL-2 (100-600 IU/mL) for 48 hours. T cells were then modified with CRISPR/Cas9 editing prior to transduction.

The PG13 packaging cell line producing retrovirus for anti-Lewis-Y were seeded at a density of 1.5×10⁶ cells/T175 in 22 mL of supplemented RPMI three days prior to harvesting viral supernatant. Non-coated 6-well plates (Corning Costar, New York, USA) were coated with 1.5 mL of 15 μg/mL retronectin (Takara, Japan) overnight at 4° C. Plates were washed three times with PBS prior to use. Viral supernatants were filtered through an Acrodisc 0.45 μM filter (Sigma-Aldrich) and 5 mL/well was added to the Retronectin-coated plates and centrifuged at 1000×g for 1 hour. Activated PBMCs were resuspended at 5×10⁵ cell/mL in 5 mL of fresh viral supernatant containing IL-2 (600 IU/mL) and added to the wells. Plates containing cells were centrifuged at 1000×g for 1 hour and incubated overnight at 37° C. This processed was repeated the next day using fresh Retronectin-coated plates. Following transduction, cells were cultured at a density of 2×10⁶ cells/mL in RPMI supplemented with 10% FCS, penicillin/streptomycin, sodium pyruvate, glutamax, NEAA and HEPES and 600 IU/mL IL-2. Transduced cells were rested and the expression of CAR was assessed via flow cytometry 3-7 days after the transduction process.

### Example 2—Identification of Genes Encoding Tumor-Specific Factors

### Murine RNA-Seq

2×10⁵ E0771-Her2 tumor cells were injected into C57BL/6 Her2Tg mice. At day 7 post-tumor injection, tumor-bearing mice were treated with total body irradiation (4 Gy) and 1×10⁷ anti-Her2 CAR T cells on two consecutive days. 50000 U/mouse IL-2 was administered i.p. in days 0-4 post-T cell treatment. At day 9 post-treatment, NGFR⁺CD8⁺ CAR T cells were isolated from spleen and tumors by FACS sorting. RNA was extracted using and analyzed by 3′RNA-Sequencing.

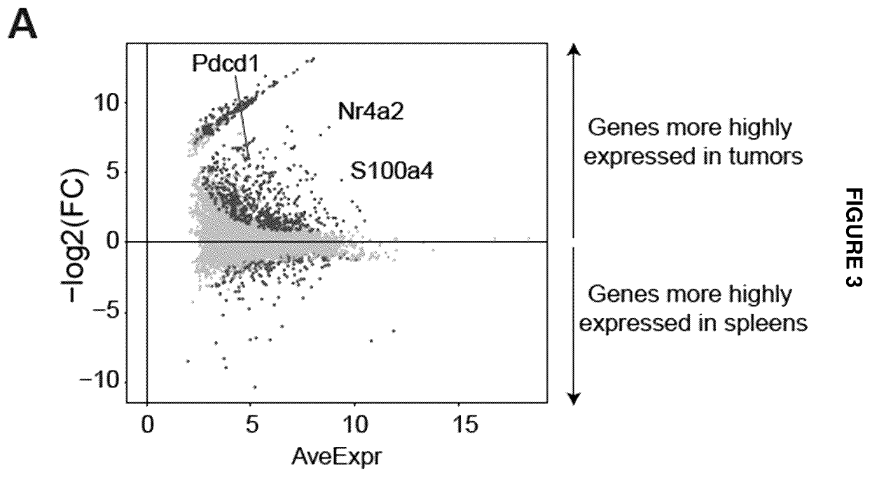

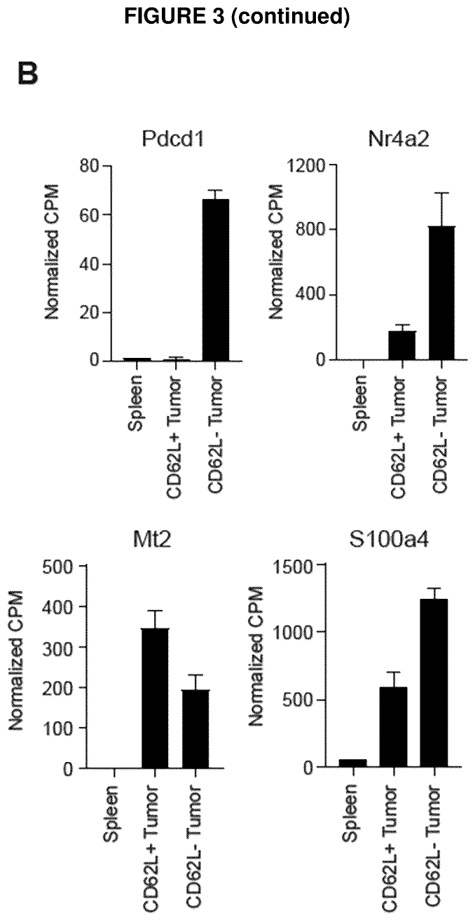

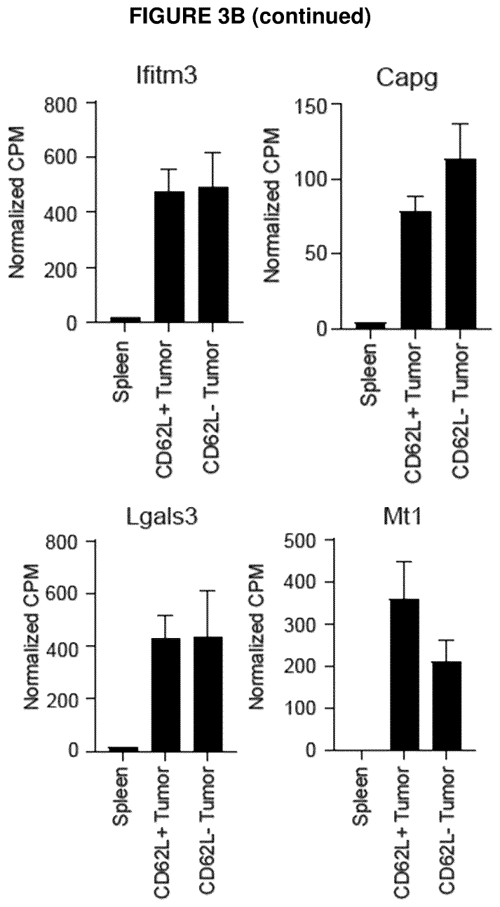

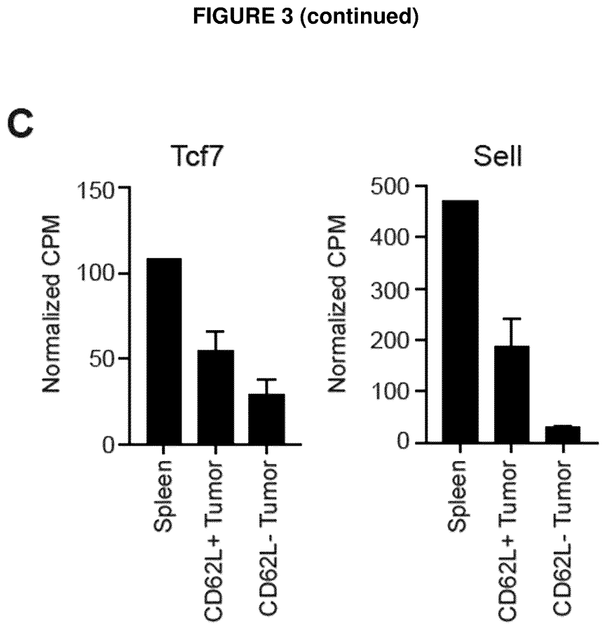

To identify genes selectively expressed at the tumor site (i.e., genes encoding tumor-specific factors), a MA plot was generated (FIG. 3). Genes with a high fold change of expression (FDR<0.05) were identified as candidate genes. Consideration for the overall level of expression was also given to ensure gene promoters were sufficiently active to generate meaningful levels of cytokines/chemokines.

### Human RNA-Seq

5×10⁶ OVCAR-3 tumor cells were injected into NSG mice. At day 14 post-tumor injection, tumor-bearing mice were treated with total body irradiation (1 Gy) and 1×10⁷ anti-Lewis Y CAR T cells on two consecutive days. 50000 U/mouse IL-2 was administered i.p. days 0-4 post-T cell treatment. At day 9 post-treatment, CD8+ and CD4+CAR T cells were isolated from spleen and tumors by FACS sorting. RNA was extracted and analyzed by 3′RNA-Sequencing.

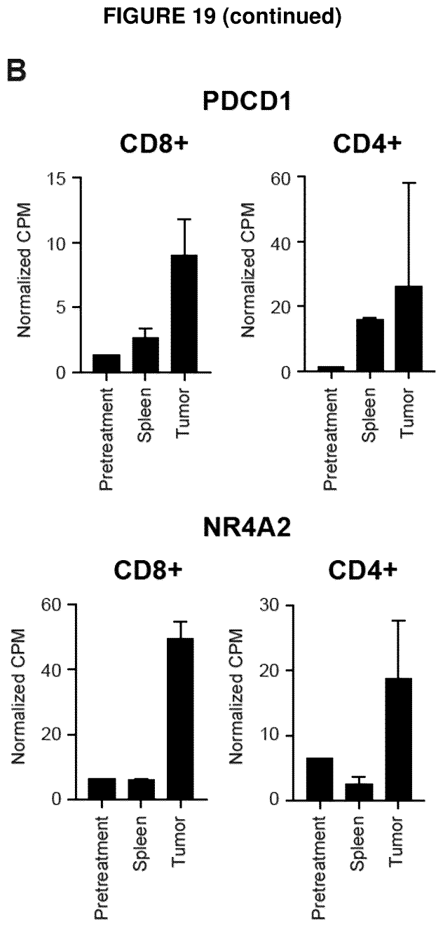

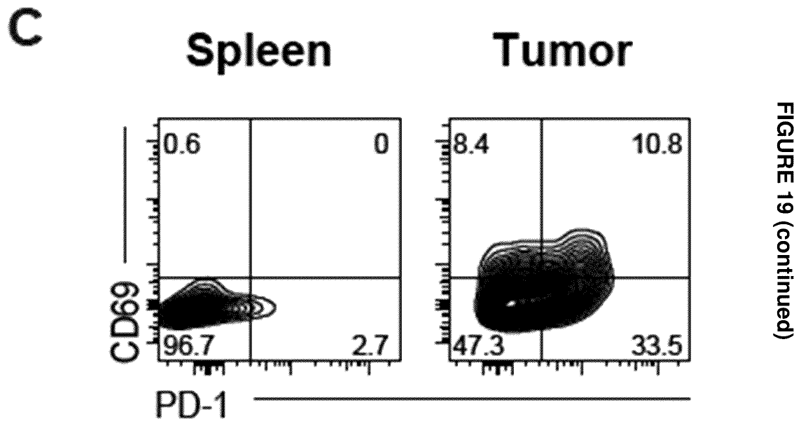

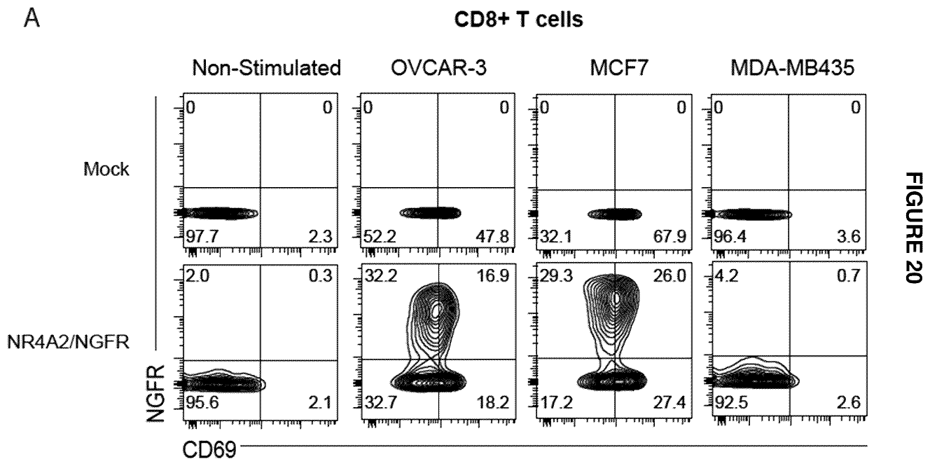

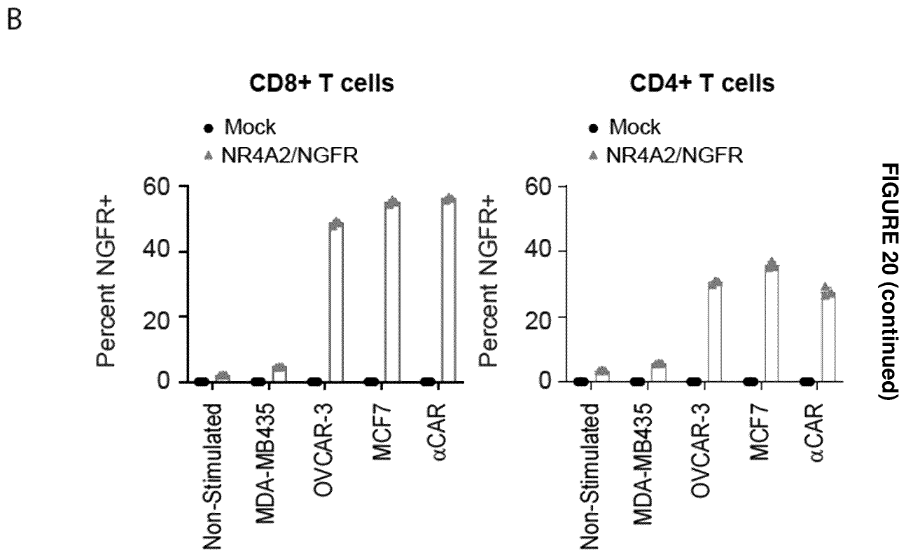

To identify genes selectively expressed at the tumor site (i.e., genes encoding tumor-specific factors), a MA plot was generated (FIG. 19A). Genes with a high fold change of expression (FDR<0.05) were identified as candidate genes. Consideration for the overall level of expression was also given to ensure gene promoters were sufficiently active to generate meaningful levels of cytokines/chemokines.

### Identification of Genes Encoding Tumor-Specific Factors

The human and mouse data were analyzed together to identify genes that were significantly increased in tumors as compared to spleen. Initially, genes significantly increased in human tumor-infiltrating CD8⁺ and CD4⁺ CAR T cells relative to their splenic counterparts were identified. Thereafter, the candidate genes encoding tumor-specific factors were further triaged on the basis of genes that were significantly increased in murine CAR T cells isolated from tumors relative to the spleen. The genes encoding tumor-specific factors identified to be significant in all comparisons are shown in Table 1.

### Example 3—sgRNA and HDR Template Design

### Disruption of Target Gene Expression

To design sgRNAs suitable for CRISPR/Cas9-mediated knock in at a site within the genes identified in Table 1, Cas9 ‘NGG’ protospacer adjacent motif (PAM) were identified within 20 base pairs of the translation start codon of the gene. Guide sequences were then designed to comprise a 20 bp RNA sequence complementary to the DNA sequence immediately 5′ of the PAM site. Guide sequences were triaged according to their on-target and off-target score calculated as described by Doench et al. (2016, Nature Biotechnology, 34: 184-191). To further interrogate possible off-target editing effects, guide sequences were assessed through the COSMID tool (Cradick et al., 2014, Molecular Therapy—Nucleic Acids, 3(12) e214) and any predicted off-target sites considered using the UCSC genome browser (Kent et al., 2002, Genome Research, 12(6): 996-1006) to ensure that potential off-target sequences were not in coding regions of genes.

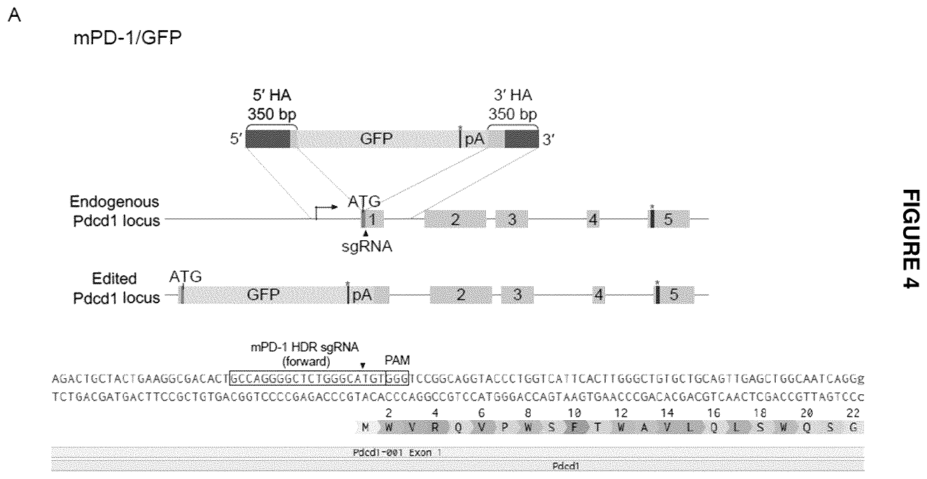

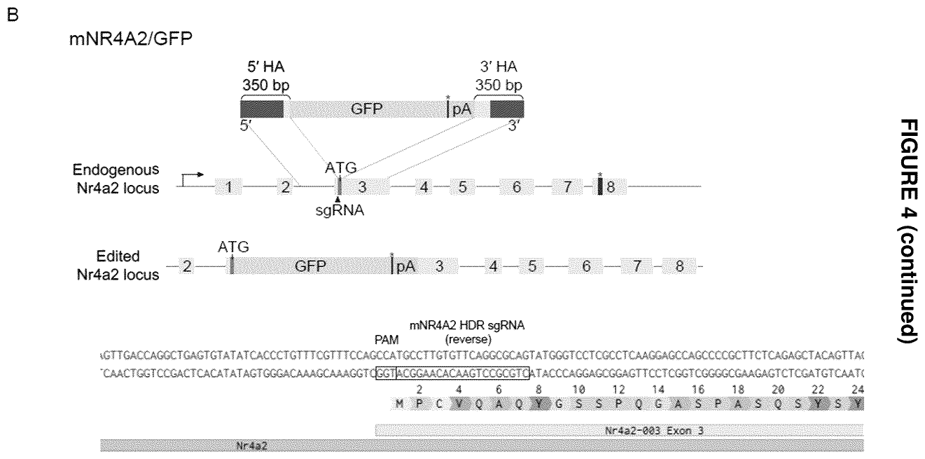

Exemplary sgRNAs are shown in Table 2 and FIG. 4.

Following this guide design, homology directed repair (HDR) templates for disruption of the expression of the endogenous gene were constructed comprising the following components, from 5′ to 3′:
(a) a 350-500 bp 5′ homology arm, homologous to the 350-500 bp genomic DNA region immediately 5′ to the translation start codon of the endogenous gene;
(b) the cDNA sequence of the transgene to be inserted (e.g., GFP, exogenous immunomodulatory factor), provided in frame with the endogenous gene expressing a tumor-specific factor (e.g., PD-1);
(c) a stop codon;
(d) a sequence coding for a poly(A) tail; and
(e) 350-500 bp 3′ homology arm, homologous to the 350-500 bp genomic DNA region immediately 3′ to the translation start codon of the endogenous gene.

Exemplary HDR templates are shown in Table 3, with the features of (a)-(e) also schematically presented in FIG. 4.

### Preservation of Target Gene Expression

Where the target gene is essential/beneficial CAR T cell function, sgRNA and homology template arms were designed to preserve expression of the endogenous gene. Accordingly, the heterologous nucleotide sequence encoding at least one immunomodulatory factor is inserted downstream of a P2A or T2A linker sequence inserted prior to the stop codon of the endogenous gene. In this variation, HDR templates for preservation of endogenous gene expression were constructed comprising the following components, from 5′ to 3′:
(a) a 350-500 bp 5′ homology arm, homologous to the 350-500 bp genomic DNA region immediately 5′ to the stop codon of the endogenous gene;
(b) a P2A or T2A linker sequence, provided in-frame of the endogenous gene;
(c) the cDNA sequence of the transgene to be inserted (e.g., GFP, exogenous immunomodulatory factor), provided in-frame with the endogenous gene expressing a tumor-specific factor (e.g., PD-1); and
(d) a 350-500 bp 3′ homology arm, homologous to the 350-500 bp genomic DNA region immediately 3′ to the stop codon of the endogenous gene.

### Example 4—Optimization of CRISPR-HDR Reagent Delivery

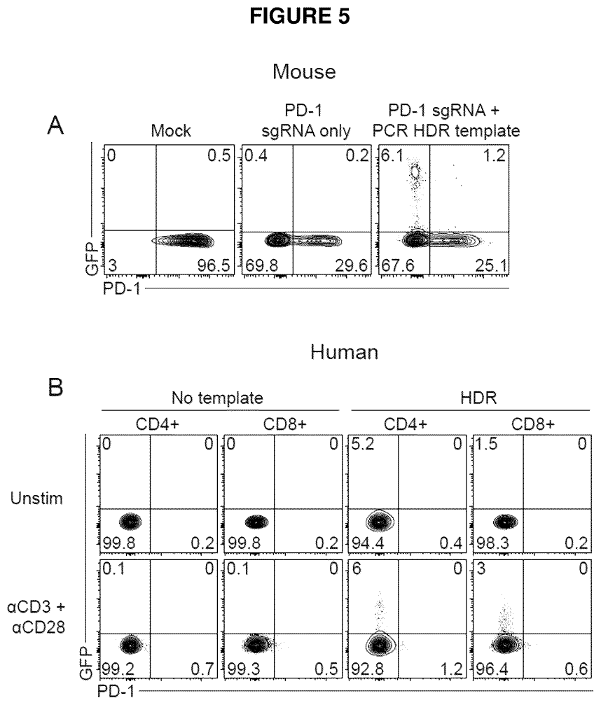

Editing efficiency of the HDR templates was assessed using PCR-generated double stranded DNA (dsDNA) repair templates in primary murine and human T cells. Briefly, CD8⁺ T cells were isolated from OT-I spleens and electroporated in the presence of a Cas9/PD-1 sgRNA RNP and PD-1/GFP HDR template. Following electroporation, T cells were cultured in media supplemented with 10 nM SIINFEKL peptide, 100 U/mL IL-2 and IL-7 for 72h before detecting GFP expression by flow cytometry (FIG. 5A). These data demonstrate the successful editing of murine primary T cells, albeit with a HDR efficiency of 3-5% in cells electroporated with sgRNA and HDR template.

For human cells, peripheral blood mononuclear cells (PBMCs) were activated with soluble anti-CD3+anti-CD28 for 72 hours, before electroporating in the presence of a Cas9/PD-1 sgRNA RNP and PD-1/GFP HDR template. Electroporated T cells were cultured for 72h before stimulating cells with soluble anti-CD3+anti-CD28 for 24 hours to upregulate PD-1 expression, then the phenotype of CD4⁺ and CD8⁺ T cells analyzed by flow cytometry (FIG. 5B). Again, successful editing was observed, with a HDR efficiency of 3-6% observed in cells electroporated with sgRNA and HDR template, as compared to HDR efficiencies of 0% and 0.1% observed in unstimulated control cells and cells electroporated with sgRNA only, respectively.

### AAV-Mediated HDR Template Delivery

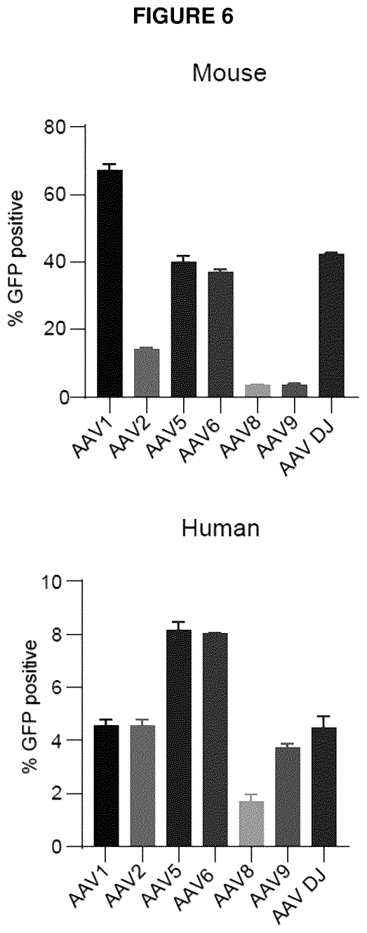

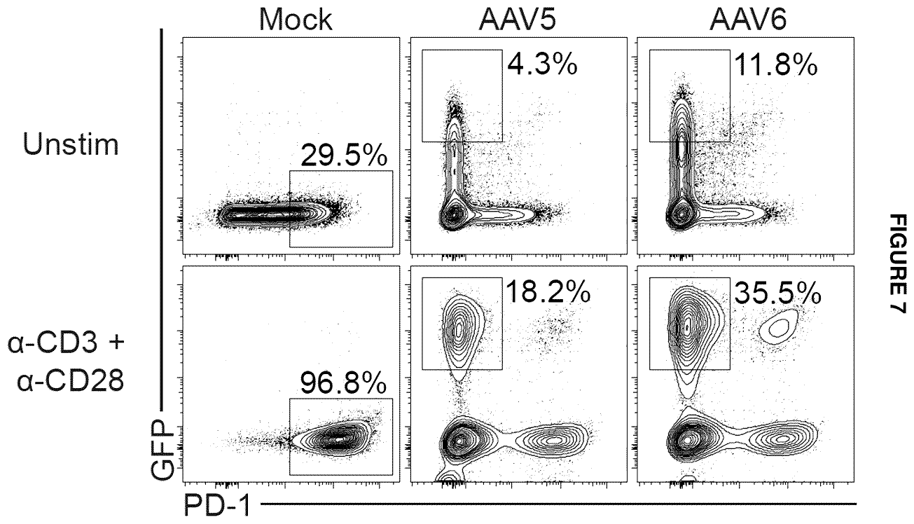

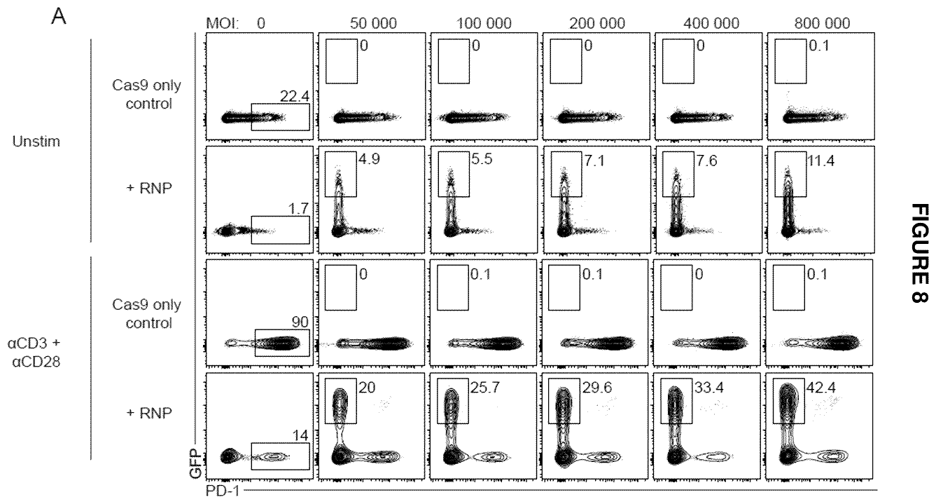

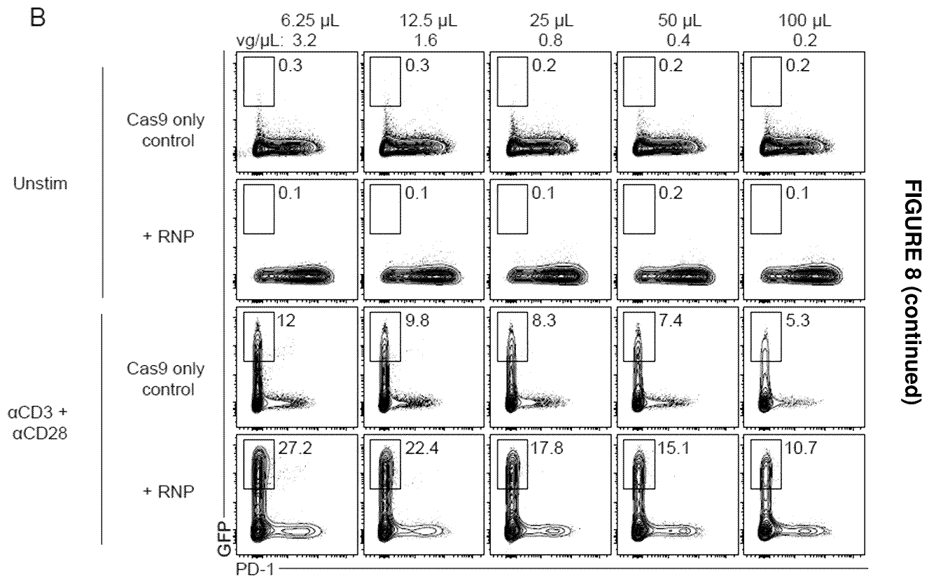

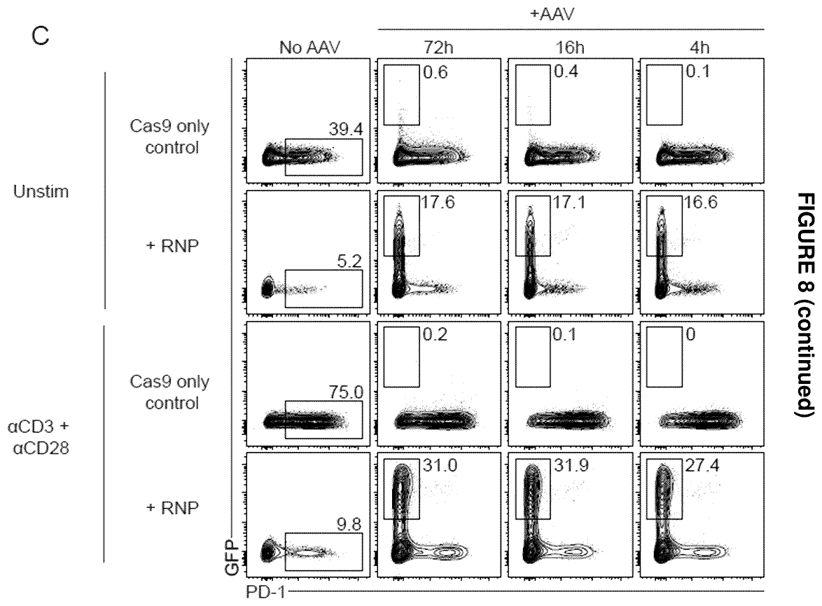

To improve HDR editing efficiency, the HDR template was introduced into cells using an adeno-associated virus (AAV) vector. Initially, the optimal AAV vector was selected from a panel of AAV serotypes. Briefly, C57BL/6 splenocytes were activated overnight with soluble anti-CD3/anti-CD28, IL-2 and IL-7. Human PBMCs were activated with anti-CD3 and IL-2 for 48 hours. Activated T cells were then electroporated and transduced with a panel of AAV serotypes encoding a CMV-GFP construct at 200,000 MOI. GFP expression in CD3⁺TCRβ⁺ cells was analyzed by flow cytometry after 24 hours culture (FIG. 6). While some level of editing was observed with each of the AAV serotypes considered, the use of AAV5 and AAV6 enable efficient transduction of both murine and human T cells.

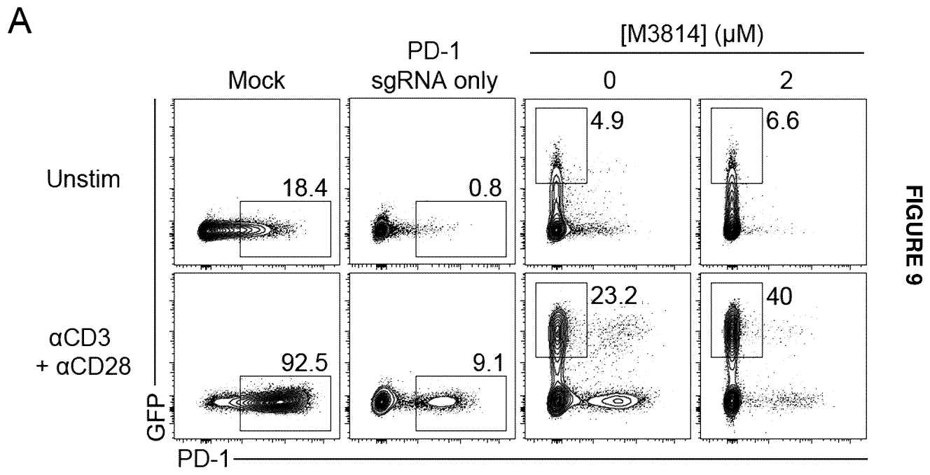

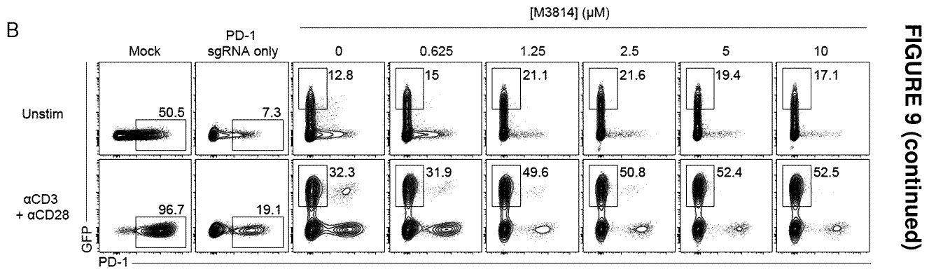

To further evaluate the use of AAV-mediated delivery of HDR templates, activated OT-I T cells were electroporated in the presence of a Cas9/PD-1 sgRNA RNP and transduced with either AAV5 or AAV6 encoding the PD-1/GFP HDR repair template. 6 days later, T cells were stimulated with plate-bound anti-CD3+anti-CD28for 24h before analyzing GFP expression by flow cytometry. As shown in FIG. 7, transduction with AAV5 or AAV6 increases HDR efficiency to more than 18% and 35%, respectively.

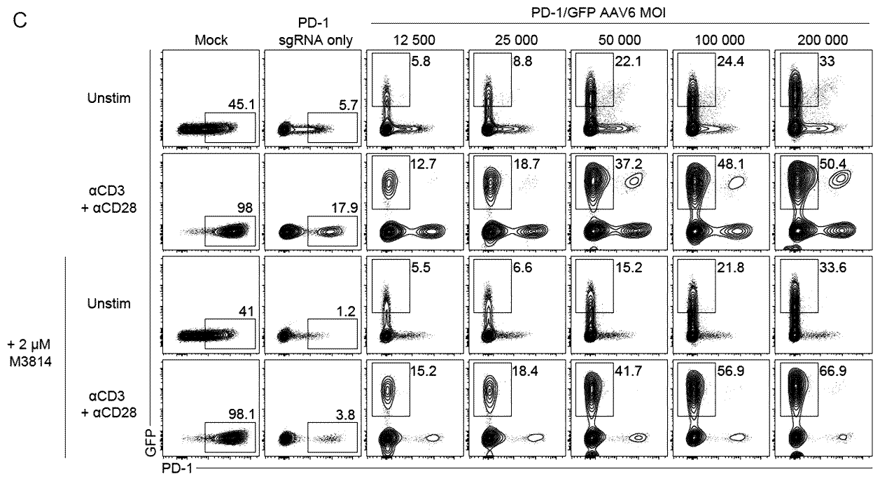

Transduction optimization of AAV6-mediated delivery of HDR templates was progressed to determine the optimal multiplicity of infection (MOI). Briefly, T cells were isolated from C57BL/6 splenocytes by overnight stimulation with soluble anti-CD3, anti-CD28, IL-2 and IL-7, then electroporated with Cas9/PD-1 sgRNA RNP or non-complexed Cas9. Electroporated T cells were incubated with AAV6 encoding the PD-1/GFP HDR template at MOIs of 50,000, 100,000, 200,000, 400,000, or 800,000 for 4 hours at 37° C. As shown in FIG. 8, editing of T cells with at least 100,000 MOI AAV6 results in >35% HDR efficiency.

Thereafter, optimal infection volume was assessed by incubating electroporated T cells with AAV6 200,000 MOI for 4 h, 16h or 72h before washing off AAV6. T cells were cultured for 6 days then stimulated overnight with plate-bound anti-CD3+anti-CD28, before analyzing by flow cytometry. As shown in FIG. 8, incubation of cells for at least 4 h increases GFP expression, particularly following stimulation with anti-CD3 and anti-CD28.

The AAV6 vector used for the remainder of the experiments described herein is shown in SEQ ID NO: 3. HDR templates were cloned into the AAV6 vector through NotI digestion and ligation.

### Non-Homologous End-Joining Inhibition

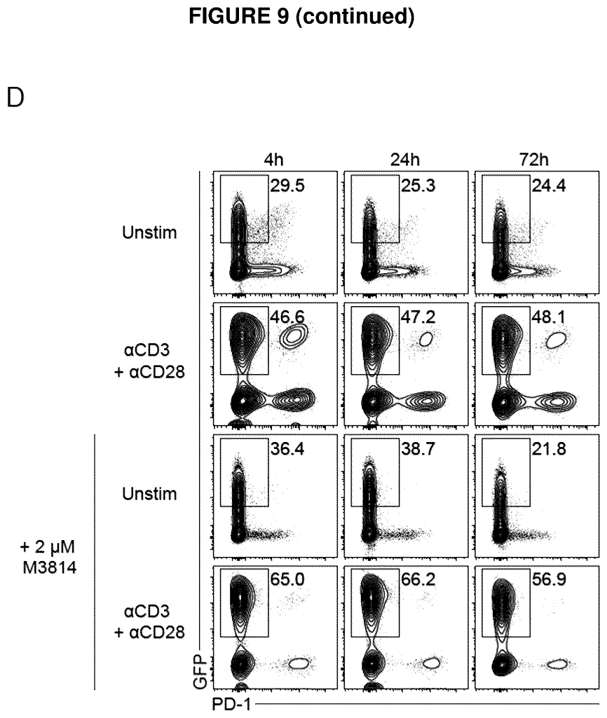

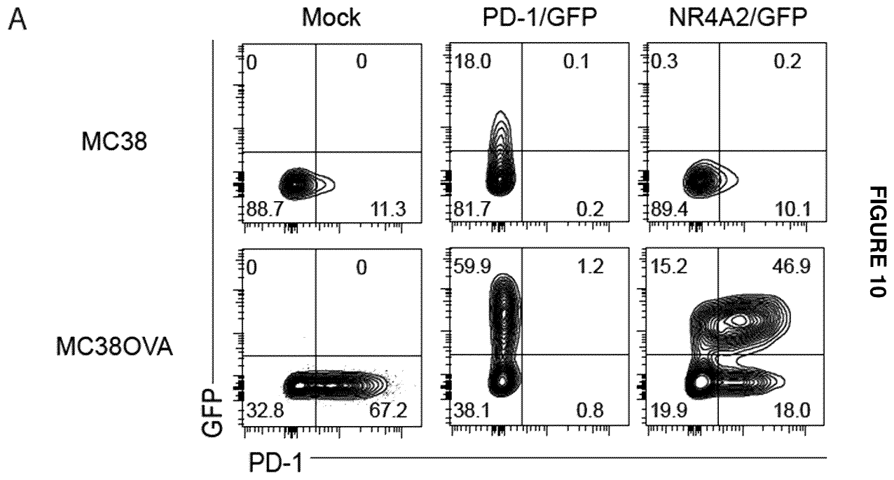

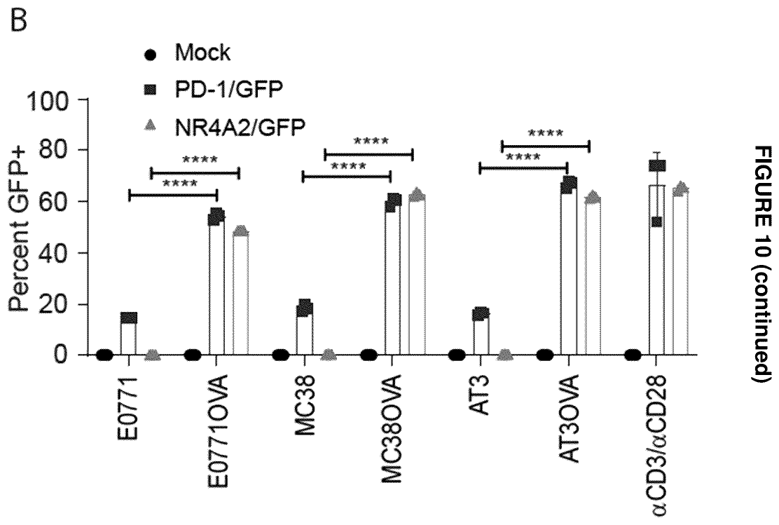

Previous studies have shown that HDR efficiency can be further improved through the use of non-homologous end-joining (NHEJ) inhibitors (see, e.g., Riesenberg et al., 2019, Nucleic Acids Research, 47(9): e116; Fu et al., 2021, Nucleic Acids Research, 49(2): 969-985). On this basis, the optimized parameters for the AAV6-mediated delivery of HDR templates was combined with the use the NHEJ inhibitor, M3814, which is an inhibitor of DNA-PK catalytic activity. Activated OT-I cells were electroporated with Cas9/PD-1 sgRNA RNP or non-complexed Cas9 and incubated with AAV6 encoding the PD-1/GFP HDR template at an MOI of 100,000 and M3814 at the indicated concentrations for 4 h at 37° C., with overnight pre-treatment with 2 μM M3814 prior to electroporation (FIG. 9A) or without pre-treatment (FIG. 9B). The OT-I cells were washed after the 4h incubation and cultured in the appropriate concentration of M3814 for a further 72h. Thereafter, electroporated OT-I cells were incubated with the PD-1/GFP AAV6 for 4h across a range of different MOIs with or without 2 μM M3814 (FIG. 9C). Again, the OT-I cells were washed after the 4h incubation and cultured in the appropriate concentration of M3814 for a further 72h. (FIG. 9C). As shown in FIGS. 9B and 9C, incubation of cells transduced with AAV6 and M3814 further increased HDR efficiency to >50%.

Additionally, electroporated OT-I cells were incubated with M3814 for 4 h (i.e., for the duration of AAV6 transduction), 24 h or 72 h. Following HDR editing, OT-I cells were cultured for a further 48-72h then stimulated with plate-bound anti-CD3 and anti-CD28 before analyzing GFP expression on live CD8+ Vu2+ cells by flow cytometry. As shown in FIG. 9A-C, when stimulating the Mock sample with anti-CD3+anti-CD28, PD-1^{high} T cells were observed, indicating PD-1 induction following stimulation with anti-CD3+anti-CD28. When stimulating the HDR-edited T cells, PD-1-GFP^{high} T cells were observed, indicating successful editing of T cells, with a HDR efficiency of >60% in cells incubated with M3814 for 4 h and 24 h (FIG. 9D).

### Example 5—Tumor-Responsive Expression of Exogenous Immunomodulatory Factors by CRISPR-HDR Edited T Cells

The optimized CRISPR/Cas9-mediated delivery method (i.e., electroporation of Cas9/sgRNA RNP followed by transduction of AAV comprising the HDR template) was used to edit murine and human T cells to express GFP, NGFR, TNF or CXCL9.

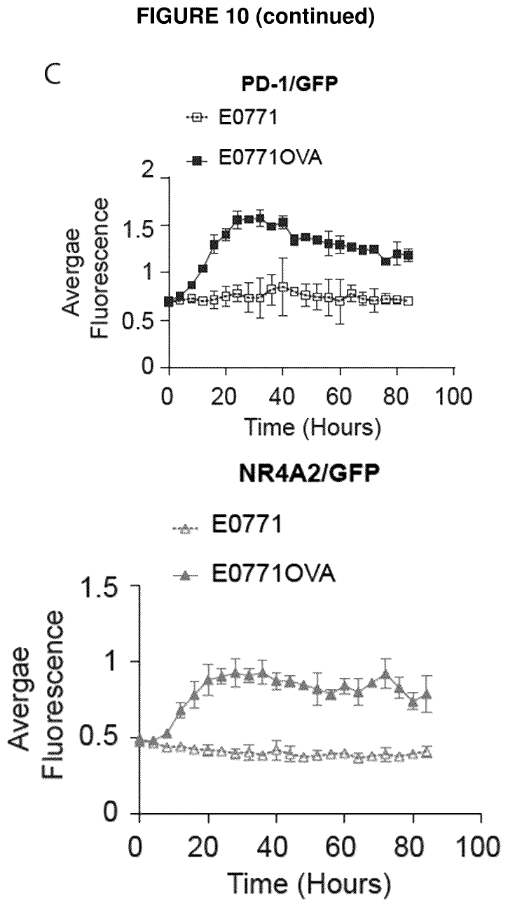

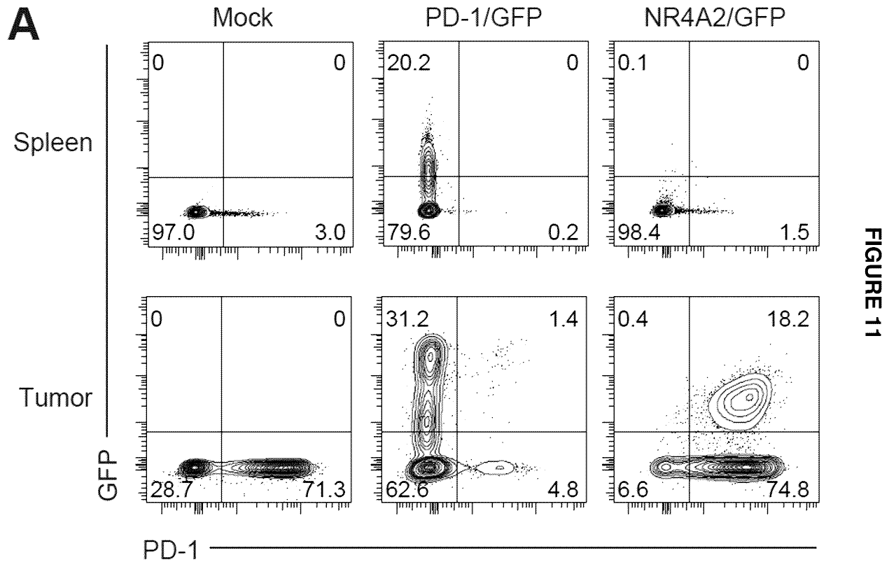

Initially, PD-1 or NR4A2 were selected as target loci to validate the approach of using tumor-specific regulatory elements to drive expression of exogenous immunomodulatory factors at the tumor site. Accordingly, OT-I T cells were activated for 24 hours with anti-CD3, anti-CD28, IL-2 and IL-7. Live cells were obtained through Ficoll centrifugation and then electroporated with Cas9 and either PD-1 or NR4A2 sgRNA. Electroporated T cells were incubated with AAV6 encoding the PD-1/GFP or NR4A2/GFP HDR templates at an MOI of 100,000 and 2 μM M3814 for 4 hours. HDR-edited OT-I cells were subsequently cultured in IL-2 and IL-7 for 4-6 days prior to co-culturing with the parental colorectal cancer cell line, MC38 or the OVA-expressing colorectal cancer cell line, MC38ova. After 24 hours the expression of PD-1 and GFP on CD8+ T cells was determined. As shown in FIG. 10A, integration of GFP into the PD-1 and NR4A2 target locus results in enhanced expression of GFP following co-culture with ova-expressing tumor cells. For both the PD-1 and NR4A2 loci, >60% GFP expressing cells were observed.

When expanded across a panel of additional tumor cell lines, including the parental E0771 (breast carcinoma cell line) and AT3 (breast carcinoma cell line) lines, together with corresponding ova-expressing lines, the expression of GFP was significantly increased following co-culture with ova-expressing tumor cells (FIG. 10B). The expression of GFP was also shown to be sustained for more than 3 days (FIG. 10C).

### TNF

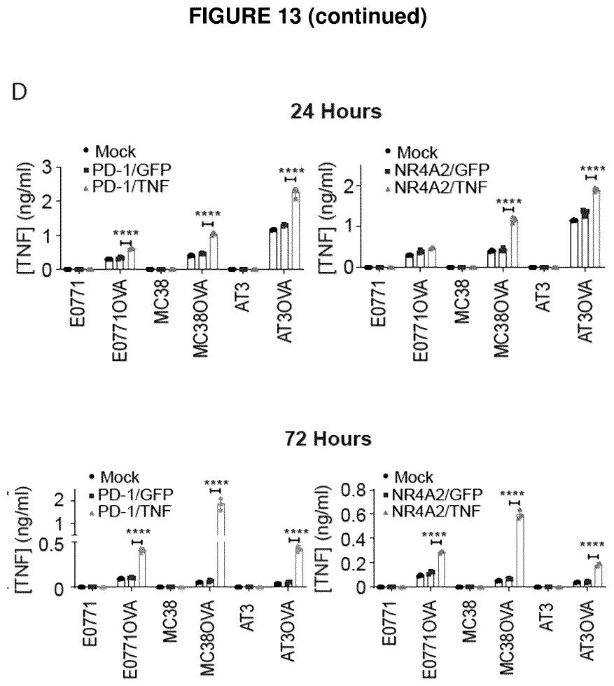

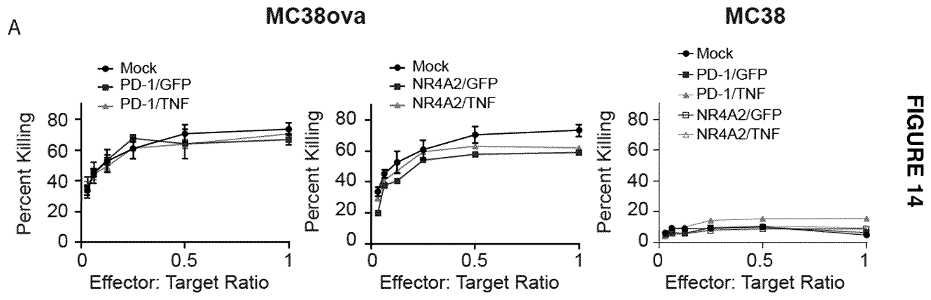

Tumor necrosis factor (TNF)-mediated tumor killing is a key mechanism that mediates the efficacy of adoptive cell transfer, and TNF can directly induce apoptosis in tumor cells (e.g., bystander killing). However, TNF is often downregulated in dysfunctional and/or exhausted T cells (see, e.g., Forucade et al., 2010, Journal of Experimental Medicine, 207(10): 2175-2186; Philip et al., 2017, Nature, 545: 452-456). T cell exhaustion is characterized by the stepwise and progressive loss of T-cell function over time. In the context of CAR T cell therapy, characteristics of T cell exhaustion include the complete loss of interferon-γ (IFNγ) and TNF production/secretion. This is reflected in vivo, with a progressive loss of IFNγ and TNF production over time. For example, in E0771-Her2 tumor bearing mice treated with anti-Her2 CAR T cells, IFNγ and TNF expression on CD8+NGFR⁺ CAR T cells reduces over time (FIG. 13A). Therefore, replacing an exhaustion gene with TNF in T cells (e.g., CAR T cells), may be useful in maintaining TNF expression over time.

To assess if exogenous TNF can be expressed by CRISPR-HDR edited immune cells under the control of the PD-1 or NR4A2 promoter, OT-I cells were activated for 24 hours with αCD3, αCD28, IL-2 and IL-7. Live cells were obtained through Ficoll centrifugation and then electroporated with Cas9 and either PD-1 or NR4A2 sgRNA. Electroporated T cells were incubated with AAV6 encoding the PD-1/TNF or NR4A2/TNF HDR templates at an MOI of 100,000 and 2 nM M3814 for 4 hours. HDR-edited OT-I cells were subsequently cultured in IL-2 and IL-7 for 4-6 days prior to 24 hours co-culture with tumor cell lines or anti-CD3/anti-CD28 as a positive control. 5×10⁴ parental MC38 or MC38ova tumor cells. CD8⁺ T cells were then analyzed for their expression of PD-1 and TNF by flow cytometry. As shown in FIGS. 13B and C, enhanced expression of TNF was observed in CRISPR-HDR edited OT-I T cells following co-culture with OVA-expressing tumor cells. For the PD-1 loci, >45% TNF expressing cells were observed. For the NR4A2 loci ˜20% TNF expressing cells were observed.

When expanded across a panel of additional tumor cell lines, including the parental E0771 (breast carcinoma cell line) and AT3 (breast carcinoma cell line) lines, together with corresponding ova-expressing lines, the expression of TNF was significantly increased following co-culture with ova-expressing tumor cells (FIG. 13C). The production of TNF was also shown to be sustained for at least 72 h (FIG. 13D).

### CXCL9

CXCL9 is a key chemokine responsible for mediating T cell trafficking into tumors (House et al., 2020, Clinical Cancer Research, 26(2): 487-504). Accordingly, the localized production of CXCL9 may assist in improved recruitment of adoptively transferred immune cells (e.g., CAR T cells) and endogenous anti-tumor immune cells to the tumor site.

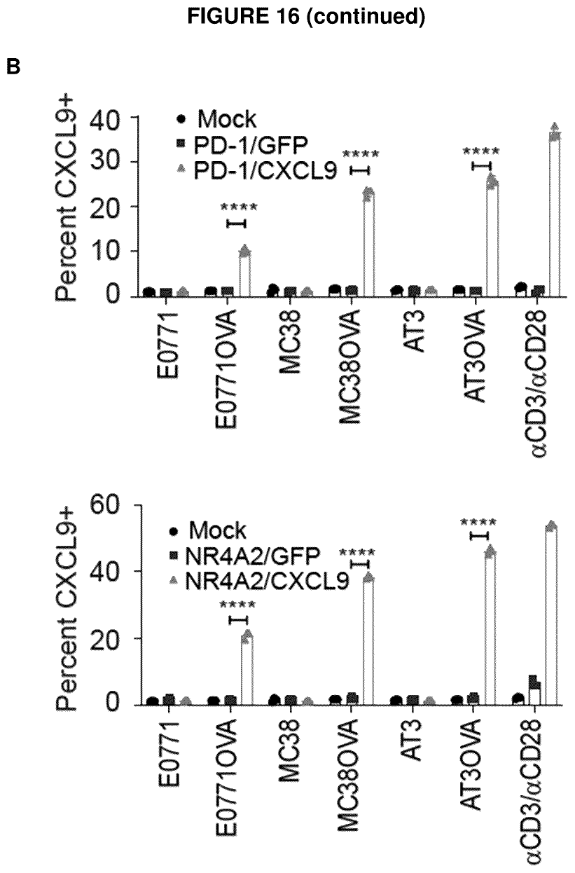

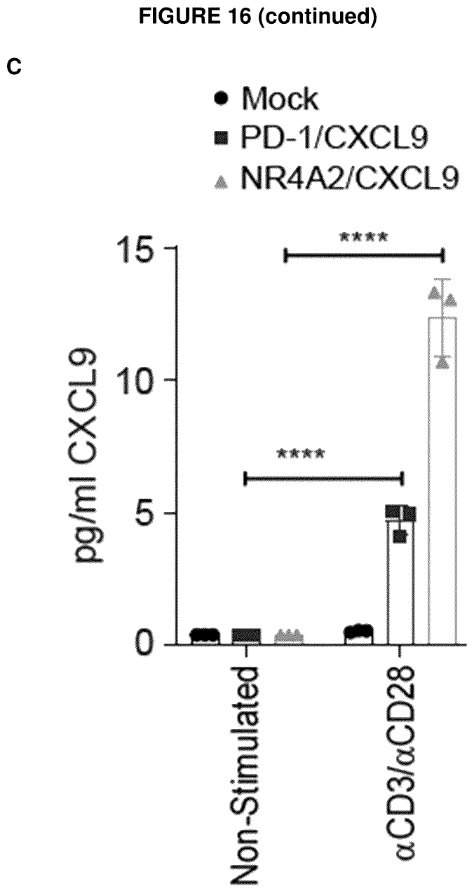

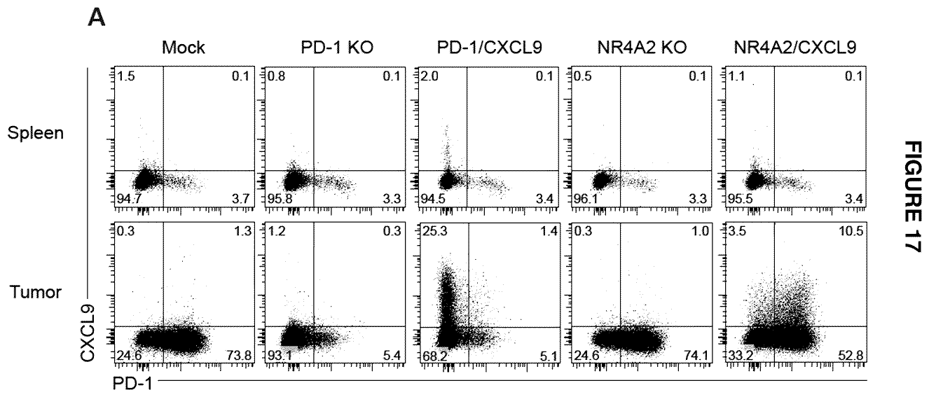

To assess if exogenous CXCL9 can be expressed by CRISPR-HDR edited immune cells under the control of the PD-1 or NR4A2 promoter, OT-I T cells were activated for 24 hours with anti-CD3, anti-CD28, IL-2 and IL-7. Live cells were obtained through Ficoll centrifugation and then electroporated with Cas9 and either PD-1 or NR4A2 sgRNA. Electroporated T cells were incubated with AAV6 encoding the PD-1/CXCL9 or NR4A2/CXCL9 HDR templates at an MOI of 100,000 and 2 μM M3814 for 4 hours. HDR-edited OT-I cells were subsequently cultured in IL-2 and IL-7 for 4-6 days prior to co-culture with tumor cell lines or anti-CD3/anti-CD28 as a positive control. 5×10⁴ OT-I T cells were co-cultured with 5×10⁴ parental MC38 or MC38ova tumor cells. After 24 hours the expression of PD-1 and CXCL9 on OT-I cells gated on CD8+ Vu2+ cells was determined. As shown in FIG. 16A, enhanced expression of CXCL9 was observed in CRISPR-HDR edited OT-I T cells following co-culture with ova-expressing tumor cells. For the PD-1 loci, >20% CXCL9 expressing cells were observed. For the NR4A2 loci ˜37% TNF expressing cells were observed.

When expanded across a panel of additional tumor cell lines, including the parental E0771 (breast carcinoma cell line) and AT3 (breast carcinoma cell line) lines, together with corresponding ova-expressing lines, the expression of CXCL9 was significantly increased following co-culture with ova-expressing tumor cells (FIG. 16B). CXCL9 was also detected in the supernatants of HDR edited T cells stimulated with anti-CD3 and anti-CD28.

### Example 6—Tumor-Responsive Expression of Exogenous Immunomodulatory Factors by CRISPR-HDR Edited CAR T Cells

To assess the tumor specificity of GFP expression controlled by the PD-1 or NR4A2 promoter in CRISPR-HDR edited CAR T cells, C57BL/6 splenocytes were activated for 24 hours with anti-CD3, anti-CD28, IL-2 and IL-7 and then CRISPR/Cas9 edited as described above in Example 4. After a 4 hour incubation at 100,000 MOI with AAV6 encoding either the PD-1/GFP or NR4A2/GFP repair template, T cells were then transduced with an anti-Her2 CAR in accordance with the methods described by Lai et al. (2020, Nature Immunology, 21: 914-926) and Giuffrida et al. (2021, Nature Communications, 12: 3236). After 5-6 days post activation, CAR T cells were either left in IL-2 and IL-7 (non-stimulated) or reactivated overnight with plate bound anti-CD3 and anti-CD28.

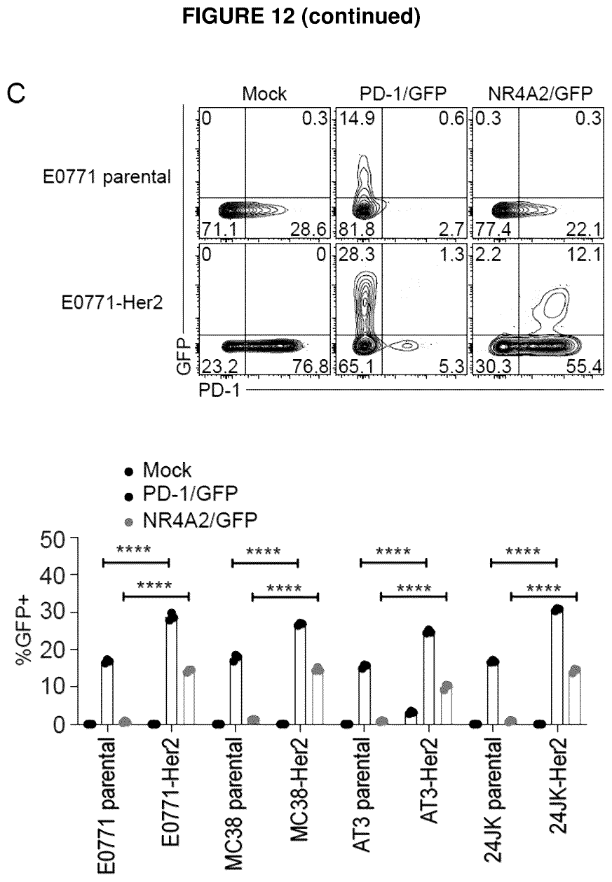

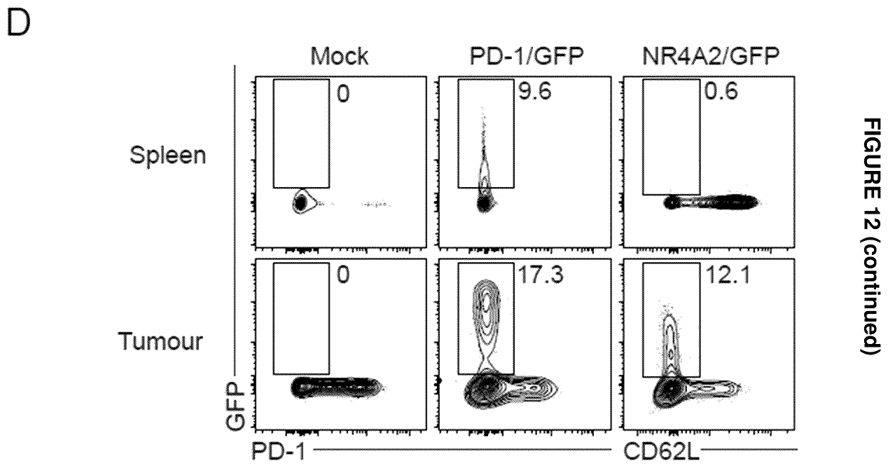

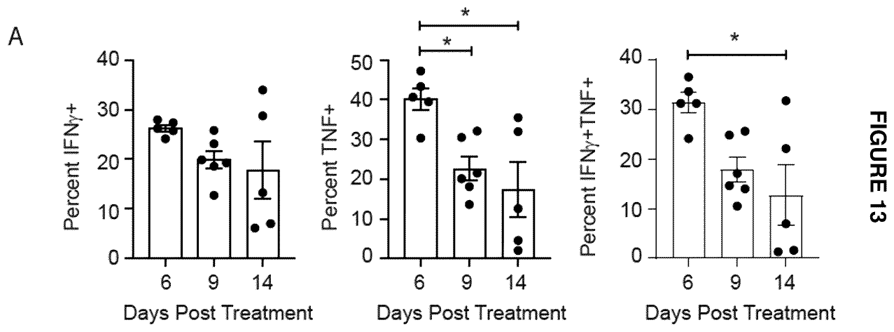

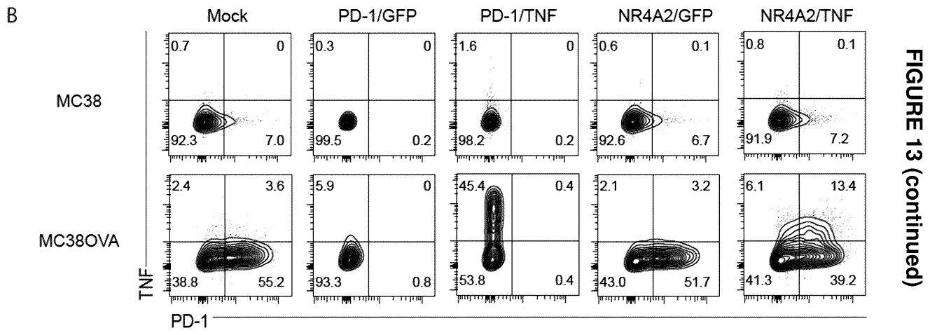

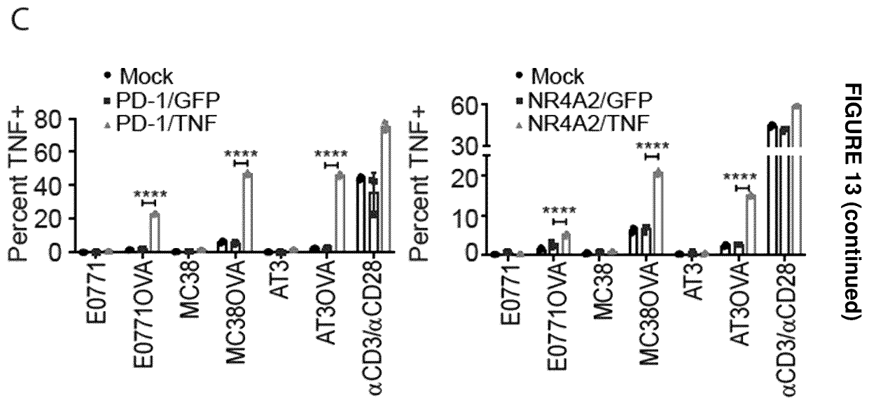

Expression of the CAR on CD8⁺ T cells was confirmed via binding of an anti-myc tag antibody (FIG. 12A). The expression of GFP was also successfully induced in PD-1/GFP in NR4A2/GFP edited CAR T cells after activation, to the same extent as HDR edited T cells that were not transduced with CAR (FIG. 12B).

Similar to the OT-I edited T cells, tumor-responsive expression of GFP was assessed by co-culturing CRISPR-HDR edited CAR T cells with Her2⁺ tumors or Her2 negative controls (parental line) at a 2:1 ratio. As shown in FIG. 12C (top panel), enhanced expression of GFP was observed in CRISPR-HDR edited CD8+ CAR T cells following co-culture with Her2-expressing tumor cells.

When expanded across a panel of additional tumor cell lines, including the parental MC38 (colon carcinoma cell line), AT3 (breast carcinoma cell line) and 24JK (sarcoma cell line) cell lines, together with corresponding Her2-expressing lines, the expression of GFP was significantly increased following co-culture with Her2-expressing tumor cells (FIG. 12C, bottom panel).

To confirm that the data presented above in relation to murine CAR T cells could be reproduced in human cells, human PBMCs were activated with soluble anti-CD3+anti-CD28 for 48 hours, before electroporating with Cas9/human NR4A2 sgRNA and then incubated for 4 hours with AAV6 encoding the human NR4A2/NGFR HDR template and 2 μM M3814. Cells were then transduced with an anti-Lewis Y CAR using the method described by Giuffrida et al. (2021, supra). After 5 days culture in IL-2, CAR T cells were used in downstream assays.

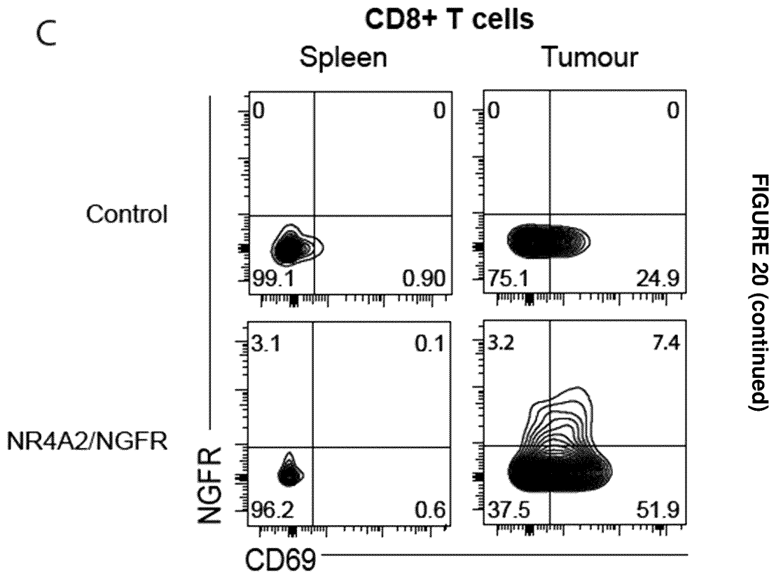

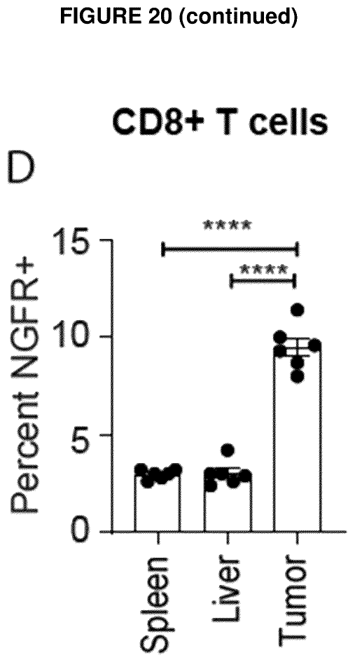

Tumor-responsive expression of NGFR was investigated by co-culturing the CRISPR-HDR edited CAR T cells with OVCAR-3 (Lewis Y positive), MCF7 (Lewis Y positive) or MDA-MB435 (Lewis Y negative) tumor cells. A plate bound anti-myc Tag (anti-CAR) antibody was used as a positive control. After 24 hours co-culture, the expression of CD69 and NGFR was determined on CD8+CAR+ and CD4+CAR+ T cells. As shown in FIGS. 20A and 20B, enhanced expression of NGFR was observed in CRISPR-HDR edited CD4+ and CD8+CAR T cells following co-culture with Lewis Y-expressing tumor cells.

### Example 7—Tumor Site-Specific Expression by CRISPR-HDR Edited T Cells

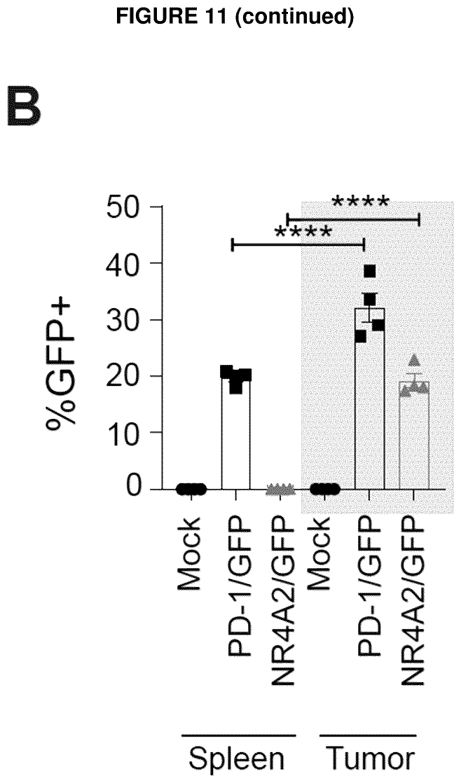

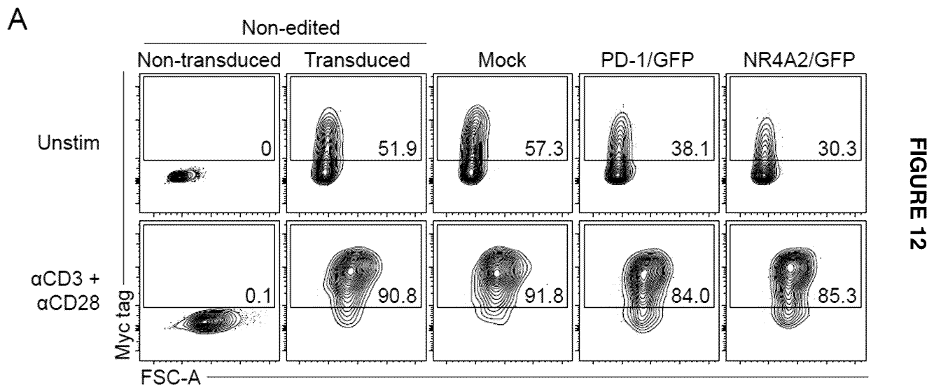

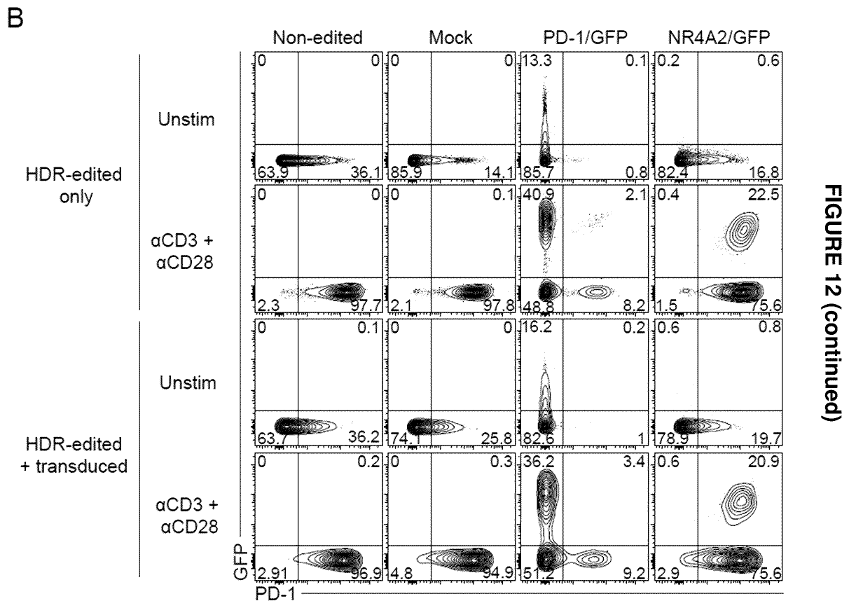

In view of the tumor-responsive expression of HDR templates observed in vitro, the tumor site-specific expression of HDR templates was assessed in vivo. 5×10⁵ E0771-OVA tumor cells were injected into the fourth mammary fat pad of Ly5.1 C57BL/6 mice. Once tumors established (20-30 mm²), mice were treated with 0.5 Gy total body irradiation and were treated with 15×10⁶ OT-I T cells edited as described above in Example 5 and subsequently cultured in IL-7 and IL-15 for 5 days. Mice were treated with 50,000 units of IL-2 per day for days 0-4 and at 7 days post-treatment, the expression of PD-1 and GFP was on CD45⁺CD8⁺Vα2⁺ cells isolated from spleens or tumors was determined by flow cytometry. As shown in FIG. 11A, enhanced expression of GFP was observed in CRISPR-HDR edited OT-I T cells isolated from the tumor, relative to spleens. Although some expression of GFP was observed in edited T cells expressing GFP under the control of the PD-1 promoter isolated from spleens, the level of expression observed in edited T cells isolated from tumor was nonetheless significantly increased (FIG. 11B).

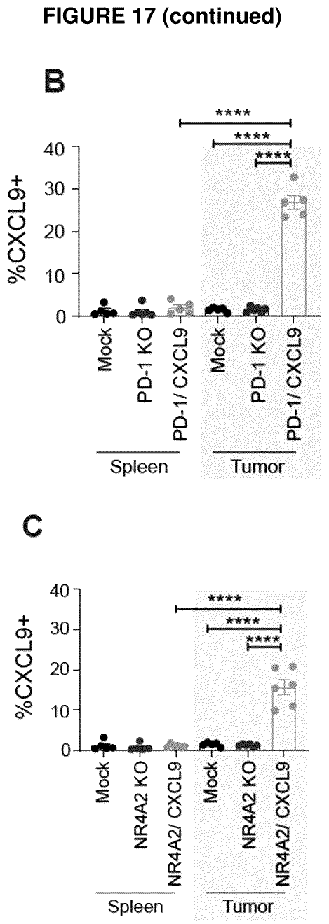

To assess if an exogenous immunomodulatory factor could also be expressed in a tumor-site specific manner, Ly5.1 mice were injected with 5×10⁵ AT3ova cells into the fourth mammary fat pad. Once tumors were established (20-30 mm²), mice were treated with total body irradiation (0.5 Gy) and 15×10⁶ OT-I cells edited as described above in Example 5, or using CRISPR/Cas9 mediated knockout of either PD-1 or NR4A2. At Day 8 post treatment, immune cell phenotype in the spleen and tumors was determined. As shown in FIG. 17A, the integration of CXCL9 into PD-1 or NR4A2 enhanced the expression of CXCL9 at the tumor site. The expression of CXCL9 was significantly increased in CD45.2+CD8+Thy+ OT-I T cells isolated from tumors relative to the equivalent cell populations isolated from spleens in both cells expressing CXCL9 under the control of the PD-1 promoter (FIG. 17B) and the NR4A2 promoter (FIG. 17D).

### Example 8—Tumor-Site Specific Expression by CRISPR-HDR Edited CAR T Cells

CRISPR-HDR edited CAR T cells were co-transduced with an NGFR expressing vector to enable detection ex vivo and were injected into mice bearing established E0771-Her2 tumors (20-30 mm²) following 4 Gy total body irradiation. Mice were treated with 50,000 units of IL-2 per day for days 0-4 and at 7 days post treatment, the expression of PD-1 and GFP was on CD8⁺NGFR⁺ cells isolated from spleens or tumors was determined by flow cytometry. As shown in FIG. 12D, the expression of GFP was increased in CD8+NGFR+ cells isolated from tumors relative to the equivalent cell populations isolated from spleens in both cells expressing GFP under the control of the PD-1 promoter and the NR4A2 promoter.

Tumor-site specific expression was also observed in human CAR T cells edited as described above in Example 6. As shown in FIGS. 20C and 20D, enhanced expression of NGFR was observed in CRISPR-HDR edited CD8+ CAR T cells isolated from tumors relative to the equivalent cell populations isolated from spleens and/or the liver.

### Example 9—Improved T Cell Activity and Therapeutic Activity of CRISPR-HDR Edited T Cells

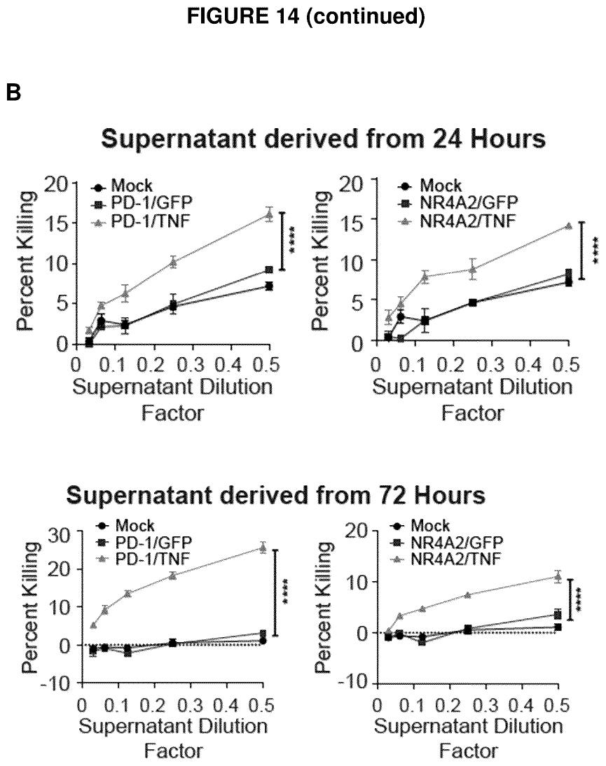

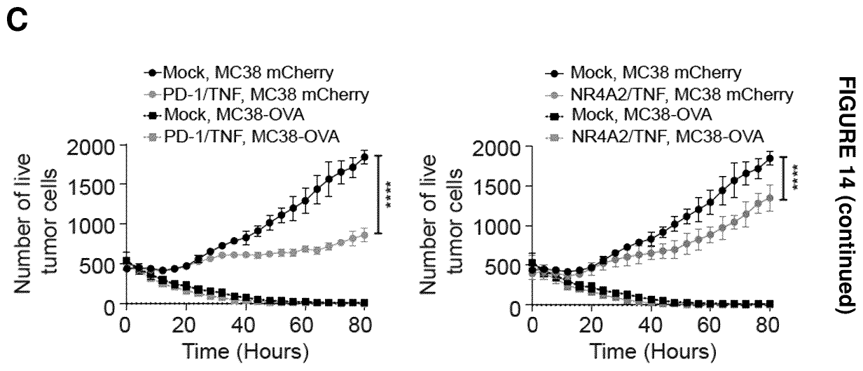

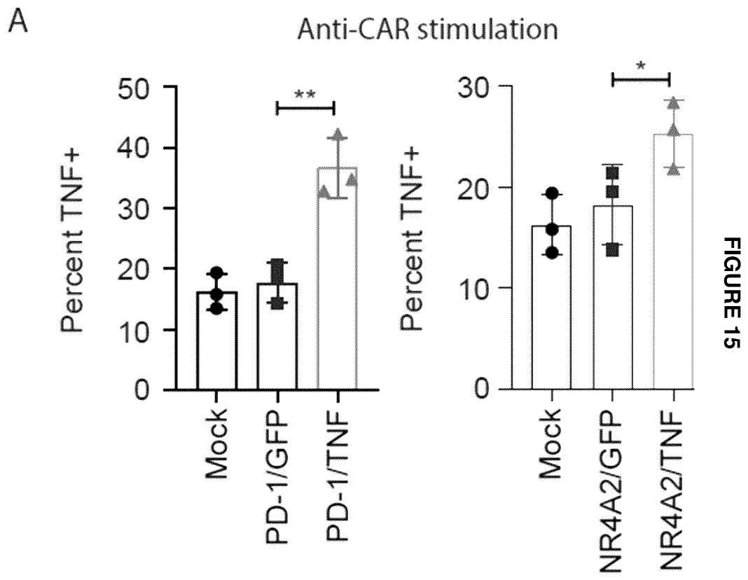

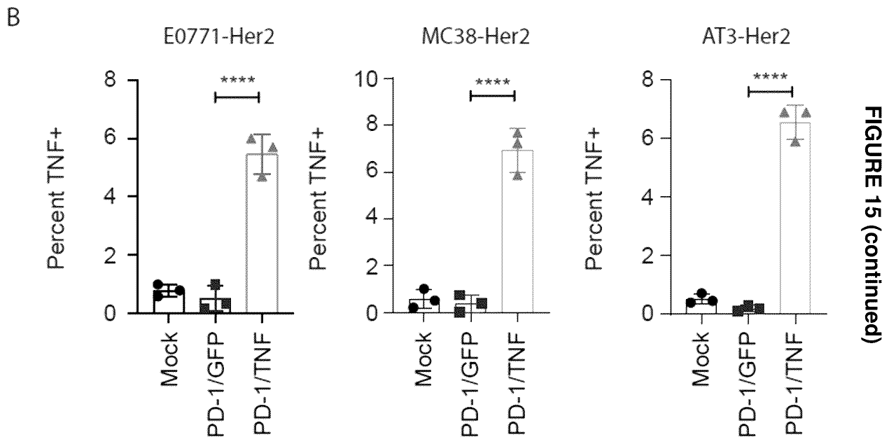

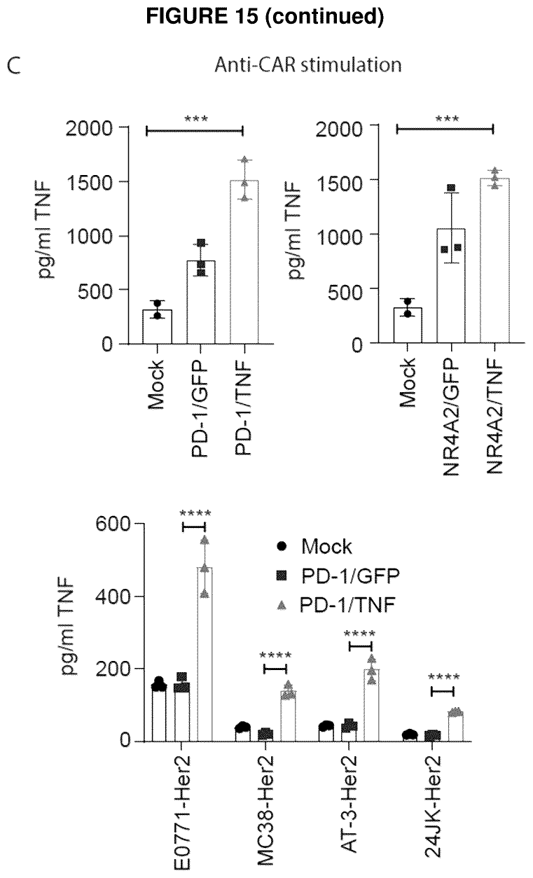

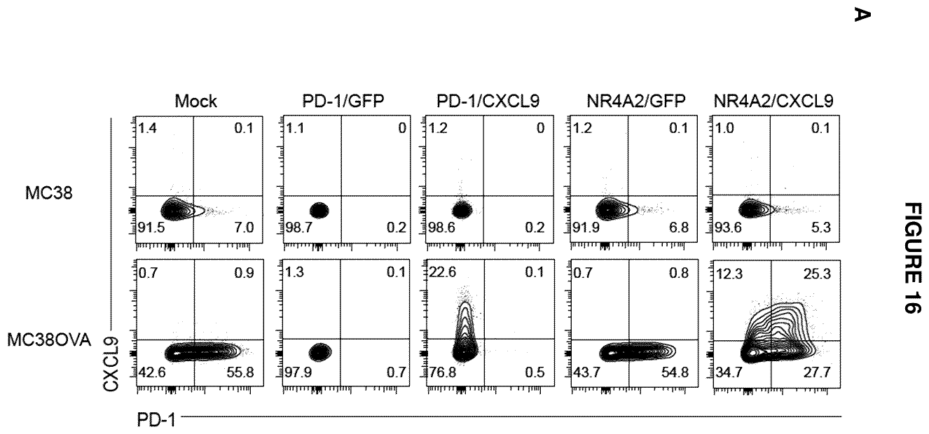

As described elsewhere herein, tumor-site specific expression of an exogenous immunomodulatory factor may contribute to the anti-tumor immune response associated with immunotherapy, e.g., by enhancing cytotoxicity, modulating the tumor microenvironment, engaging host immunity or improving lymphocyte trafficking. Accordingly, cytotoxicity of CRISPR-HDR edited T cells was assessed by culturing OT-I T cells edited to express GFP or TNF as described in Example 5 and subsequently cultured in IL-2 and IL-7 for 4-6 days. The edited OT-I T cells were then co-cultured with 1×10^{4 15}Cr labelled MC38ova tumor cells at indicated Effector: Target ratios for 4 hours. As shown in FIG. 14A, direct tumor killing by the edited OT-I T cells was largely equivalent in cells expressing GFP and TNF following co-culture with OVA-expressing tumor cells. However, when supernatants from the co-culture of OT-I T cells with MC38ova tumor cells were then applied to MC38 tumor cells labelled with ⁵¹Cr for 16 hours to assess indirect tumor killing (i.e., the bystander killing assay), enhanced cytotoxicity was observed in cells expressing TNF. In particular, supernatants taken at 24 hours and 72 hours from co-cultures with edited OT-I T cells expressing TNF resulted in significantly higher cytotoxicity relative to mock edited or edited OT-I T cells expressing GFP (FIG. 14B).

The improved cytotoxicity of parental MC38 tumors as observed by the chromium release assay in 14B was confirmed by live cell imaging killing assays, which quantified live MC38ova and MC38 mCherry cells in a mixed coculture with edited OT-I T cells (FIG. 14C). Of note, the ability of the PD-1/TNF and NR4A2/TNF edited OT-I cells to eliminate parental MC38 tumor cells (antigen-negative) demonstrated that T cell activation by the MC38ova tumor cells led to sufficient TNF production through PD-1 and NR4A2 to elicit effective TNF-mediated bystander killing.

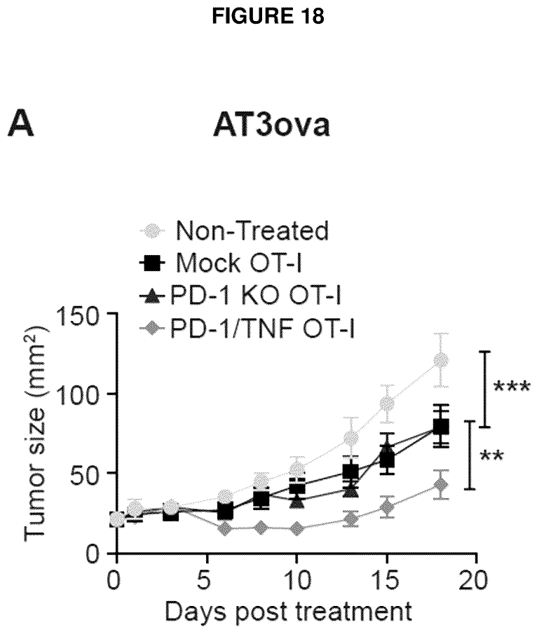

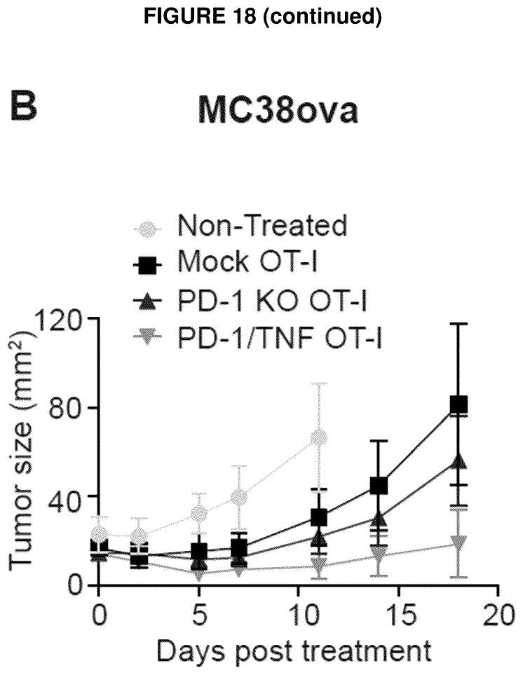

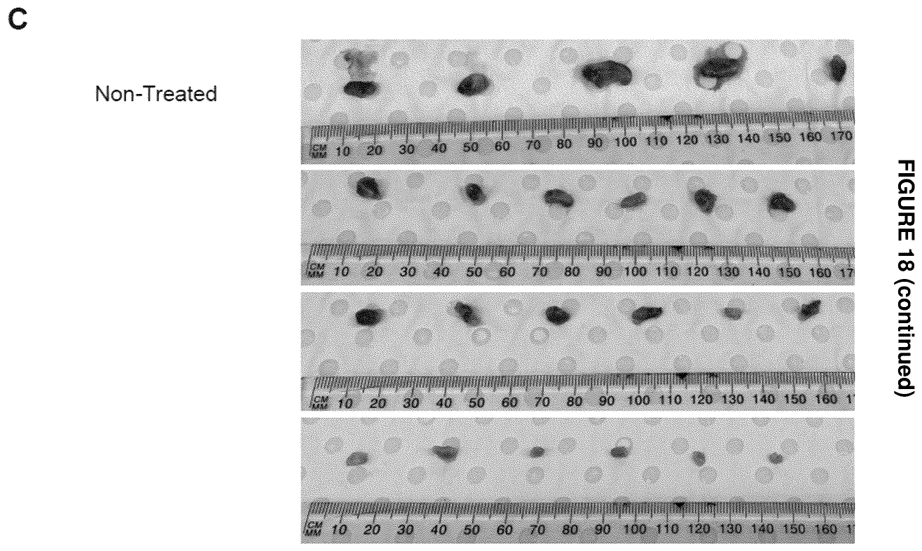

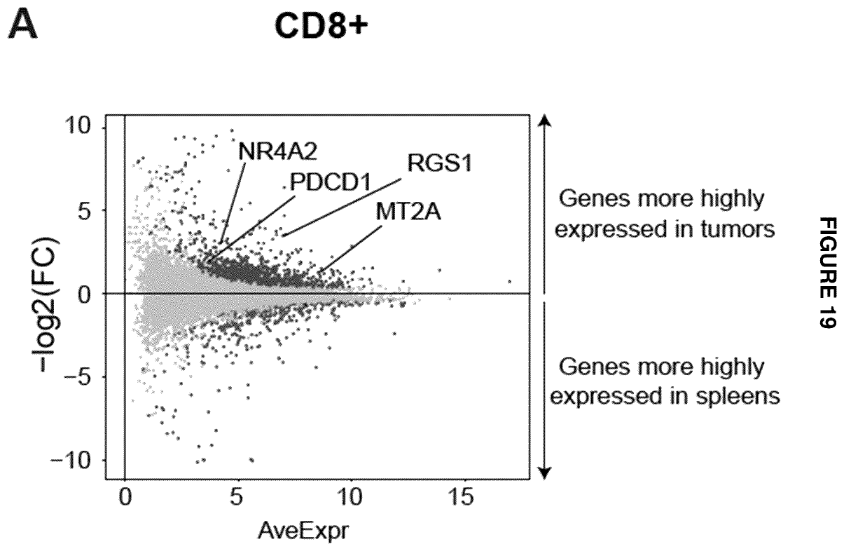

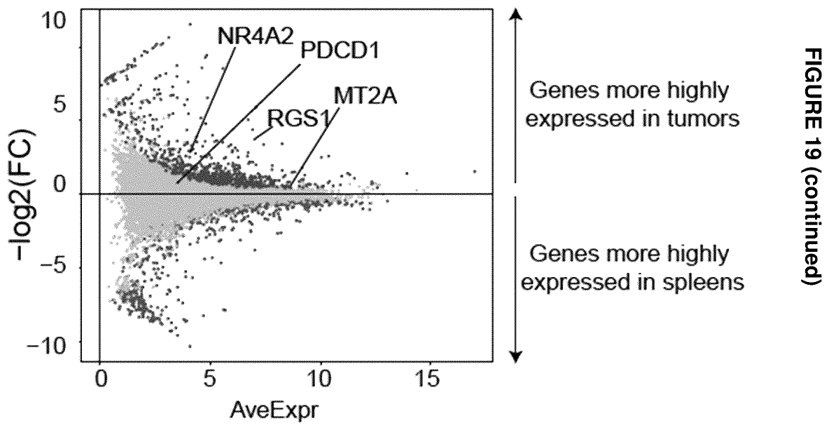

Integration of TNF at the PD-1 locus was also shown to enhance therapeutic activity in vivo. Briefly, C57BL/6 or Ly5.1 mice were injected with either 5×10⁵ AT3ova cells into the fourth mammary fat pad or 1×10⁶ MC38ova cells sub-cutaneously. Once tumors were established (20-30 mm²) mice were treated with total body irradiation (0.5 Gy) and 15×10⁶ or 5×10⁶ OT-I cells edited using CRISPR/Cas9 mediated knockout or CRISPR-HDR as described above in Example 5. Mice were treated with IL-2 (50,000 U/mouse) daily for days 0-4 post T cell treatment. As shown in FIG. 18, tumor size was significantly reduced following treatment with CRISPR-HDR edited OT-I T cells expressing TNF under the control of the PD-1 promoter.

### DISCUSSION

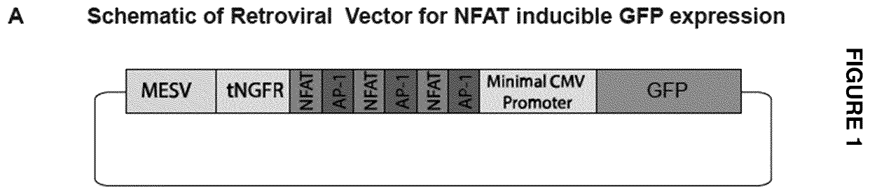

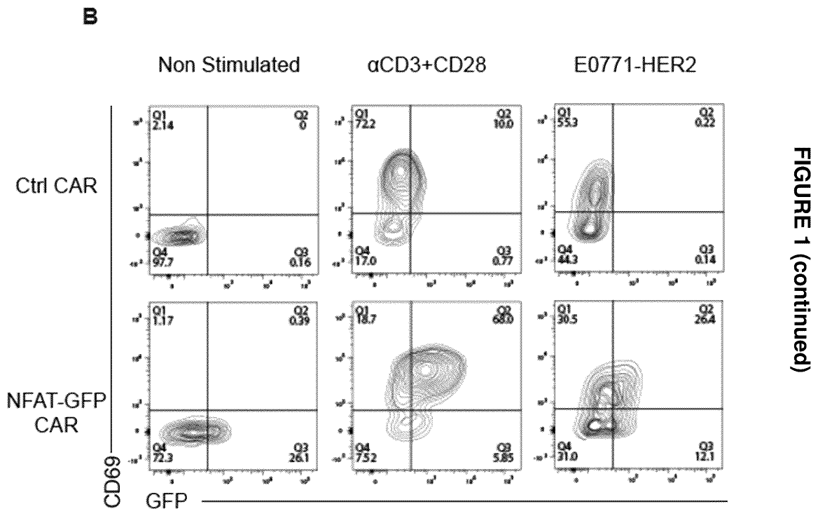

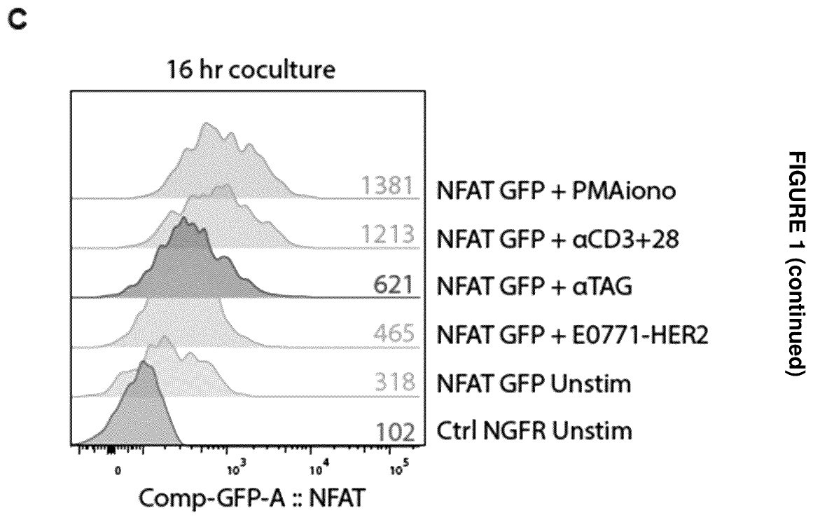

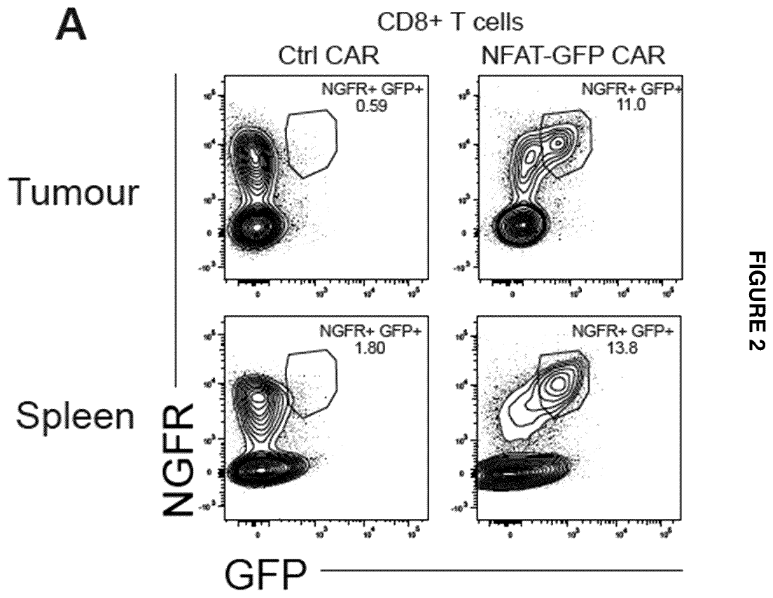

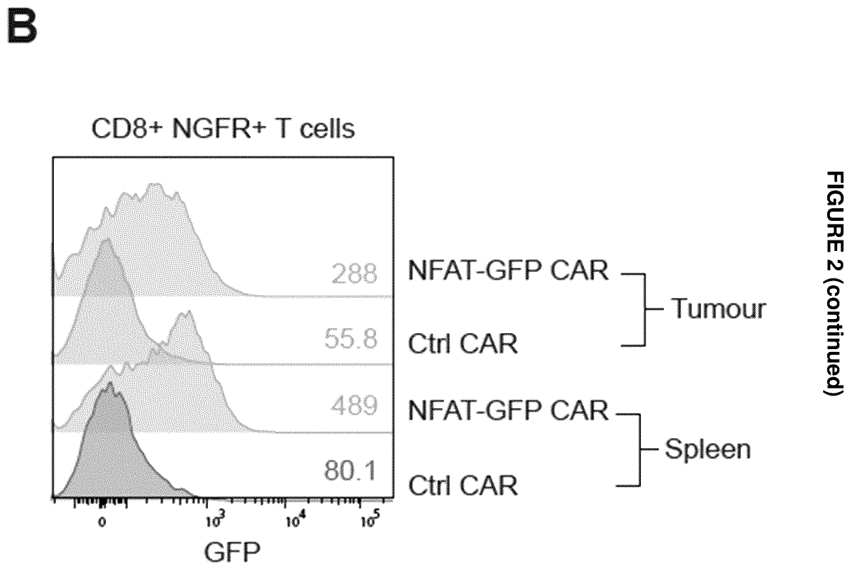

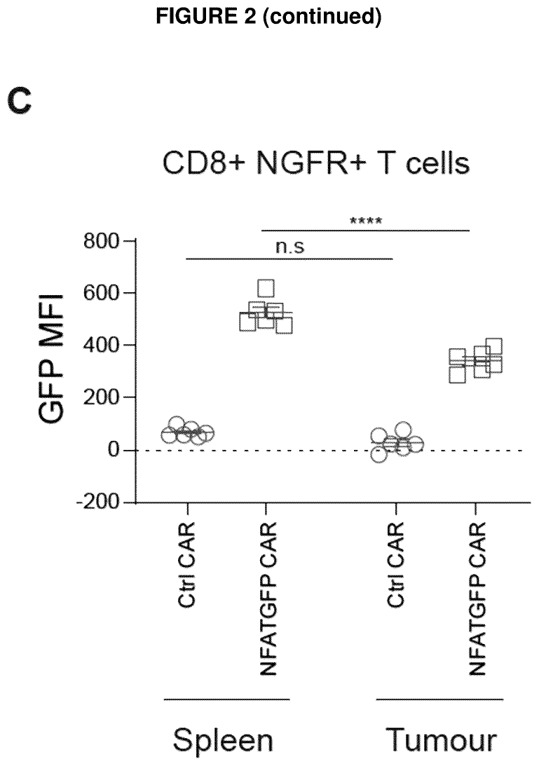

Collectively, these data are enabling of engineered T cells that express exogenous immunomodulatory factors in a tumor-site specific manner. Unlike previous approaches to the generation of “armored” T cells for use in immunotherapy, the engineered T cells of the present disclosure to not constitutively express exogenous immunomodulatory factors, thereby avoiding the toxicity that has been observed in previous studies. For example, the engineered T cells of the present disclosure exhibit more specific expression at the tumor site as compared to NFAT promoter systems that have been previously used for the generation of armored CAR T cells (see, e.g., FIGS. 1 and 2).

Accordingly, it has surprisingly been shown that the introduction of a heterologous nucleotide sequence encoding at least one immunomodulatory factor in-frame of an endogenous gene of the T cells encoding a tumor-specific factor under the control of endogenous regulatory elements, wherein expression of the heterologous nucleotide sequence is controlled by the endogenous regulatory elements of the endogenous genes enables the tumor-site specific expression of the immunomodulatory factor(s) encoded by heterologous nucleotide sequence, which improves anti-tumor T cell function and therapeutic efficacy.

TABLE 1
Genes encoding tumor-specific factors
Genes

| | |
|---|---|
| AK4 | IFNG |
| ALDOA | ITPRIPL2 |
| APLP2 | JUN |
| BHLHE40 | JUNB |
| BST2 | KLRC1 |
| C15orf48 | KLRC2 |
| CD69 | KRT8 |
| CD9 | LAPTM4B |
| CDKN1A | LDHA |
| CHAF1A | MT1E |
| CLU | MT1F |
| COL6A3 | MT2A |
| CSF1 | MX1 |
| CSRNP1 | MYO10 |
| CTLA4 | MYO1E |
| CXCR6 | NFIL3 |
| DUSP4 | NFKBIA |
| E2F8 | NR4A2 |
| EGR1 | PDCD1 |
| EIF2AK2 | PDE4A |
| FNDC3B | PDLIM4 |
| FOS | PGAM1 |
| FOSB | PHLDA1 |
| FURIN | PKM |
| GAPDH | PMEPA1 |
| HAVCR2 | PPP1R15A |
| HIF1A | RAI14 |
| HIP1 | RBPJ |
| RGS1 | SLCO4A1 |
| RGS16 | SOCS1 |
| RGS2 | STAT3 |
| RGS9 | TGFBI |
| RHOC | TNF |
| RNF213 | TNFAIP3 |
| SAMSN1 | TPI1 |
| SDC4 | TPM4 |
| SEMA7A | TUBB6 |
| SH2D2A | XCL1 |
| SKIL | ZMIZ1 |
| SLC16A3 | |

TABLE 2
Exemplary sgRNAs

| Target | sgRNA | SEQ ID NO |
|---|---|---|
| Mouse PD-1 | GCCAGGGGCUCUGGGCAUGU | 4 |
| Mouse NR4A2 | CUGCGCCUGAACACAAGGCA | 5 |
| Human PD-1 | UCCAGGCAUGCAGAUCCCAC | 6 |
| Human NR4A2 | GCCUGAACACAAGGCAUGGC | 7 |

TABLE 3
Exemplary HDR templates

| HDR template | SEQ ID NO |
|---|---|
| Murine PD-1/GFP | 8 |
| Murine NR4A2/GFP | 9 |
| Murine PD-1/CXCL9 | 10 |
| Murine NR4A2/CXCL9 | 11 |
| Murine PD-1/CXCL10 | 12 |
| Murine NR4A2/CXCL10 | 13 |
| Murine PD-1/CXCL9-P2A-CXCL10 | 14 |
| Murine NR4A2/CXCL9-P2A-CXCL10 | 15 |
| Murine PD-1/TNF | 16 |
| Murine NR4A2/TNF | 17 |
| Murine PD-1/IFNg | 18 |
| Murine NR4A2/IFNg | 19 |
| Murine PD-1/TNF-P2A-INFg | 20 |
| Murine NR4A2/TNF-P2A-INFg | 21 |
| Human PD-1/NGFR | 22 |
| Human NR4A2/NGFR | 23 |
| Human PD-1/CXCL9 | 24 |
| Human NR4A2/CXCL9 | 25 |
| Human PD-1/CXCL10 | 26 |
| Human NR4A2/CXCL10 | 27 |
| Human PD-1/CXCL9-P2A-CXCL10 | 28 |
| Human NR4A2/CXCL9-P2A-CXCL10 | 29 |
| Human PD-1/TNF | 30 |
| Human NR4A2/TNF | 31 |
| Human PD-1/IFNg | 32 |
| Human NR4A2/IFNg | 33 |
| Human PD-1/TNF-P2A-INFg | 34 |
| Human NR4A2/TNF-P2A-INFg | 35 |

TABLE 4
Exemplary heterologous nucleotide sequences
encoding immunomodulatory factors

| Immunomodulatory factor | SEQ ID NO |
|---|---|
| Human NGFR | 36 |
| Human CXCL9 | 37 |
| Human CXCL10 | 38 |
| Human CXCL9-P2A-CXCL10 | 39 |
| Human TNF | 40 |
| Human IFNg | 41 |
| Human TNF-P2A-IFNg | 42 |

1. An engineered T cell comprising a heterologous nucleotide sequence encoding at least one immunomodulatory factor, wherein the heterologous nucleotide sequence is introduced in-frame of an endogenous gene of the T cell encoding a tumor-specific factor under the control of endogenous regulatory elements, wherein expression of the heterologous nucleotide sequence is controlled by the endogenous regulatory elements of the endogenous gene.

2. The engineered T cell of claim 1, wherein the heterologous nucleotide sequence is introduced at a position adjacent to the translation start codon of the endogenous gene.

3. The engineered T cell of claim 2, wherein the heterologous nucleotide sequence is introduced at a position no more than about 20 base pairs upstream or downstream of the translation start codon of the endogenous gene.

4. The engineered T cell of claim 2 or claim 3, wherein introduction of the heterologous nucleotide sequence disrupts expression of the endogenous gene.

5. The engineered T cell of claim 1, wherein the heterologous nucleotide sequence is introduced at a position adjacent to the stop codon of the endogenous gene.

6. The engineered T cell of claim 5, wherein the heterologous nucleotide sequence is introduced at a position no more than about 20 base pairs upstream of the stop codon of the endogenous gene.

7. The engineered T cell of any one of claims 1 to 6, wherein the heterologous nucleotide sequence further comprises one or more or all of:
(a) a stop codon;
(b) a nucleotide sequence encoding a poly(A) tail; and
(c) a linker.

8. The engineered T cell of any one of claims 1 to 7, wherein the immunomodulatory factor is a cytokine selected from the group consisting of TNF, IFNγ, IFNα, IFNβ, IL-12, IL-18, CXCL9, CXCL10, XCL1, CD40L, and combinations of the foregoing.

9. The engineered T cell of claim 8, wherein the cytokine is selected from the group consisting of TNF, CXCL9, CXCL10, IFNγ, and combinations of the foregoing.

10. The engineered T cell of claim 9, wherein the heterologous nucleotide sequence comprises a sequence of any one of SEQ ID NOs: 36 to 42, or a sequence that is at least 80% identical to the sequence of any one of SEQ ID NOs: 36 to 42.

11. The engineered T cell of any one of claims 1 to 10, wherein the endogenous gene is selected from the genes listed in Table 1.

12. The engineered T cell of claim 11, wherein the endogenous gene is selected from PD-1 and NR4A2.

13. The engineered T cell of any one of claims 7 to 12, wherein the linker is selected from a P2A linker and a T2A linker.

14. The engineered T cell of any one of claims 1 to 13, further comprising a chimeric antigen receptor (CAR).

15. The engineered T cell of claim 14, wherein the CAR binds an antigen selected from the group consisting of CD19, CD20, CD22, CD30, ROR1, CD123, CD33, CD133, CD138, GD2, Her2, Her1, mesothelin, MUC1, gp100, MART-1, MAGE-A3, MUC16, NY-ESO-1, L1-CAM, CEA, FAP, VEGFR2, WT1, TAG-72, CD171, α-FR, CAIX, PSMA, EGFRvIII CLL-1, GRP78, claudin 6, claudin 18.2 and Lewis Y.

16. The engineered T cell of claim 15, wherein the CAR binds an antigen selected from Her2 and Lewis Y.

17. The engineered T cell of any one of claims 1 to 16 for use in the treatment of cancer.

18. A pharmaceutical composition comprising the engineered T cell of any one of claims 1 to 16.

19. A method for the treatment of cancer comprising the administration of a therapeutically effective amount of the engineered T cell of any one of claims 1 to 16 or the pharmaceutical composition of claim 18 to a subject in need thereof.

20. Use of the engineered T cell of any one of claims 1 to 16 or the pharmaceutical composition of claim 18 in the manufacture of a medicament for the treatment of cancer.

21. A genome editing system comprising:
(a) a sgRNA comprising a sequence of at least 10 contiguous nucleotides that are complementary to a target nucleic acid sequence within an endogenous gene of a T cell encoding a tumor-specific factor under the control of endogenous regulatory elements;
(b) a RNA-guided nuclease; and
(c) a homology directed repair (HDR) template, wherein the HDR template comprises a nucleotide sequence encoding at least one immunomodulatory factor.

22. The genome editing system of claim 21, wherein the RNA-guided nuclease is CRISPR-associated endonuclease 9 (Cas9).

23. The genome editing system of claim 21 or claim 22, wherein the HDR template is within a vector.

24. The genome editing system of claim 23, wherein the vector is an adeno-associated virus (AAV) vector.

25. The genome editing system of claim 24, wherein the AAV vector is an AAV5 or AAV6 vector.

26. The genome editing system of any one of claims 21 to 25, wherein the sgRNA and the RNA-guided nuclease are complexed as a ribonucleoprotein (RNP).

27. The genome editing system of any one of claims 21 to 26, further comprising an inhibitor of non-homologous end-joining (NHEJ).

28. The genome editing system of claim 27, wherein the inhibitor of NHEJ is a DNA-PK inhibitor.

29. The genome editing system of any one of claims 21 to 28, wherein the target nucleic acid sequence comprises or is adjacent to the translation start codon of the endogenous gene.

30. The genome editing system of claim 29, wherein the translation start codon of the endogenous gene is within from about 10 nucleotides to about 50 nucleotides upstream or downstream of a protospacer adjacent motif (PAM).

31. The genome editing system of claim 29 or claim 30, wherein the HDR template further comprises one or more or all of:
(a) a 5′ homology arm comprising a sequence of from about 250 to about 600 contiguous nucleotides that are homologous to a region 5′ to the translation start codon of the endogenous gene;
(b) a stop codon;
(c) a nucleotide sequence encoding a poly(A) tail;
(d) a linker; and
(e) a 3′ homology arm comprising a sequence of from about 250 to about 600 contiguous nucleotides that are homologous to a region 3′ to the translation start codon of the endogenous gene.

32. The genome editing system of claim 31, wherein the HDR template comprises a sequence of any one of SEQ ID NOs: 22 to 35, or a sequence that is at least 80% identical to the sequence of any one of SEQ ID NOs: 22 to 35.

33. The genome editing system of any one of claims 21 to 28, wherein the target nucleic acid sequence comprises or is adjacent to the stop codon of the endogenous gene.

34. The genome editing system of claim 33, wherein the HDR template further comprises one or more of all of:
(a) a 5′ homology arm comprising a sequence of from about 250 to about 600 contiguous nucleotides that are homologous to a region 5′ to the stop codon of the endogenous gene;
(b) a linker; and
(c) a 3′ homology arm comprising a sequence of from about 250 to about 600 contiguous nucleotides that are homologous to a region 3′ to the stop codon of the endogenous gene.

35. The genome editing system of any one of claims 21 to 34, wherein the immunomodulatory factor is a cytokine selected from the group consisting of TNF, IFNγ, IFNα, IFNβ, IL-12, IL-18, CXCL9, CXCL10, XCL1, CD40L, and combinations of the foregoing.

36. The genome editing system of claim 35, wherein the cytokine is selected from the group consisting of TNF, CXCL9, CXCL10, IFNγ, and combinations of the foregoing.

37. The genome editing system of any one of claims 21 to 36, wherein the endogenous gene is selected from the genes listed in Table 1.

38. The genome editing system of claim 37, wherein the endogenous gene is selected from PD-1 and NR4A2.

39. The genome editing system of any one of claims 31 to 38, wherein the linker is selected from a P2A linker and a T2A linker.

40. A method of altering a nucleic acid molecule in a T cell, the method comprising providing to the T cell the genome editing system of any one of claims 21 to 39.

41. The method of claim 40, wherein:
(a) the sgRNA and RNA-guided nuclease are provided to the T cell complexed as an RNP; and
(b) the HDR template is provided to the T cell within a vector.
